(19) United States
(12) Patent Application Publication
(10) Pub. No.: US 2014/0017262 A1
(43) Pub. Date: Jan. 16, 2014

(54) CRIPTO BINDING MOLECULES

(71) Applicant: Biogen Idec MA Inc., Cambridge, MA (US)

(72) Inventor: Michele SANICOLA-NADEL, Winchester, MA (US)

(73) Assignee: Biogen Idec MA Inc., Cambridge, MA (US)

(21) Appl. No.: 13/784,302

(22) Filed: Mar. 4, 2013

Related U.S. Application Data
(63) Continuation of application No. 12/602,625, filed on May 27, 2010, now abandoned.
(60) Provisional application No. 60/932,879, filed on Jun. 1, 2007.

(51) Int. Cl. A61K 47/48; A61K 31/5365; A61K 31/513

(52) U.S. Cl. CPC A61K 47/48384; A61K 31/513; A61K 31/5365
USPC 424/178.1

(57) ABSTRACT

The invention pertains to humanized forms of an anti-CRIPTO antibody and portions thereof and their use in treating disorders, such as cancer either alone or in combination with other agents.

### RELATED APPLICATIONS

This application claims the benefit of U.S. Ser. No. 60/932,879, titled “Cripto Binding Molecules,” filed on Jun. 1, 2007. This application is related to International Patent Application PCT/US 2006/000502, titled “Cripto Binding Molecules”, filed on Jan. 5, 2006. This application is also related to U.S. Ser. No. 60/641,691, titled “Purification and Preferential Synthesis of Binding Molecules,” filed on Jan. 5, 2005. This application is also related to U.S. Ser. No. 60/483,877, titled “Purification and Preferential Synthesis of Polypeptides,” filed on Jun. 27, 2003 and to U.S. Ser. No. 60/508,810, titled “Purification and Preferential Synthesis of Antigen Binding Polypeptides,” filed Oct. 3, 2003. This application is also related to U.S. Ser. No. 10/880,320, titled “Purification and Preferential Synthesis of Binding Molecules” filed on Jun. 28, 2004. This application is also related to U.S. Ser. No. 10/945,853, titled “Cripto-Specific Antibodies,” filed Sep. 20, 2004, to U.S. Ser. No. 10/693,538, titled “Cripto Blocking Antibodies and Uses Thereof,” filed Oct. 23, 2003, and to U.S. Application Nos. 60/367,002, titled “Antibodies Directed to the Ligand Binding Domain of Cripto,” filed Mar. 22, 2002; 60/301,091, titled “Cripto Blocking Antibodies and Uses Thereof,” filed Jun. 26, 2001; 60/293,020, titled “Antibodies Directed to the Ligand Binding Domain of Cripto,” filed May 17, 2001; and 60/286,782, titled “Antibodies Directed to the Ligand Binding Domain of Cripto,” filed Apr. 26, 2001. The contents of each of these applications are incorporated in their entirety by this reference.

### BACKGROUND OF THE INVENTION

Antibodies, and various engineered forms thereof, are effective therapeutic agents currently being used to treat patients suffering from a variety of disorders. Some of these antibodies recognize antigens present on the surface of tumor cells. Cripto is a 188-amino-acid cell surface protein overexpressed by many tumor cells. Cripto was isolated in a cDNA screen of a human embryonic carcinoma library (Ciccodicola et al., 1989, EMBO J. 8:1987-91). Cripto was originally classified as a member of the EGF family (Ciccodicola et al., supra); however, subsequent analysis showed that Cripto did not bind any of the known EGF receptors and its EGF-like domain was actually divergent from the EGF family (Bianco et al., 1999, J. Biol. Chem. 274:8624-29).

Overexpression of the Cripto protein is associated with tumors in many tissues (including, but not limited to brain, breast, testicular, colon, lung, ovary, bladder, uterine, cervical, pancreatic and stomach). Panico et al., 1996, Int. J. Cancer 65:51-56; Byrne et al., 1998, J. Pathology 185:108-11; De Angelis et al., 1999, Int. J. Oncology 14:437-40.

Murine antibodies that bind to Cripto have been described. However, while murine antibodies do have applicability as therapeutic agents in humans, because they are not of human origin they may be immunogenic. Administration of such antibodies may result in a neutralizing antibody response (human anti-murine antibody (HAMA) response), which is particularly problematic if the antibodies are desired to be administered repeatedly, e.g., in treatment of a chronic or recurrent disease condition. Also, because they contain murine constant domains they may not exhibit human effector functions.

In an effort to alleviate the immunogenicity concerns, “humanized” antibodies are often produced. In one protocol, CDRs from an antibody of mouse origin are transferred onto human framework regions resulting in a “CDR grafted” antibody. Frequently, amino acid residues which could potentially affect antigen binding in the framework region are backmuated the corresponding mouse residue.

However, while humanized antibodies are desirable because of their potential low immunogenicity in humans, their production is unpredictable. For example, sequence modification of antibodies may result in substantial or even total loss of antigen binding affinity, or loss of binding specificity. In addition, despite sequence modification “humanized antibodies” may still exhibit immunogenicity in humans. Such antibodies would provide a means for targeting Cripto positive tumor cells in order to deliver anti-tumor agents, such as toxins, radiolabels, and the like. The development of such conjugated antibody molecules and dosing regimens for administering them would be of tremendous benefit.

### SUMMARY OF THE INVENTION

The invention is based, at least in part, on the discovery that a humanized anti-Cripto antibody, B3F6.1, conjugated to a maytansoid (B3F6.1-DM4) is effective in inhibiting tumor cell growth in vivo in animal models when administered in a single dose or in a biweekly dosage regimen. The biweekly dosing in these models indicates that an effective dose of B3F6.1-DM4 in man includes a dosing regimen of administration once every 3 weeks. The invention is further based on the discovery that a single dose of B3F6.1-DM4 is effective in inhibiting growth of established tumors in an in vivo animal models. The invention is still further based on the discovery that the administration of B3F6.1-DM4 together with an additional agent, e.g., an antimetabolite, e.g., 5′-fluorouracil, results in a synergistic inhibition of tumor cell growth in vivo in an in vivo animal model.

Accordingly, in one aspect, the invention provides a method of inhibiting growth of a tumor in a subject, comprising administering to the subject an effective dose of a binding molecule which binds to Cripto, wherein the binding molecule is administered once every three weeks, thereby inhibiting growth of a tumor in a subject.

In one embodiment, the binding molecule is an anti-Cripto antibody. In one embodiment, the binding molecule is a humanized anti-Cripto antibody. In one embodiment, the anti-Cripto antibody is conjugated to a maytansoid, e.g., DM4. In one embodiment, the maytansoid is conjugated to the antibody via a heterobifunctional crosslinking agent, e.g., SPDB. In one embodiment, an average of 3.5 molecules of DM4 is attached to the anti-cripto antibody.

In one embodiment, the subject is suffering from a cancer in an organ selected from the group consisting of brain, breast, testicular, colon, rectum, lung, ovary, bladder, uterine, cervical, pancreatic and stomach. In a preferred embodiment, the subject is suffering from colon cancer.

In one embodiment, the effective dose of the binding molecule (e.g., humanized anti-Cripto antibody conjugated to a maytansinoid, e.g., B3F6.1-DM4) is selected from the group consisting of about 5 mg/kg, about 10 mg/kg, about 15 mg/kg, about 25 mg/kg and about 40 mg/kg.

In another aspect, the invention provides a method of inhibiting growth of a tumor in a subject, comprising administering to the subject an effective dosage of a binding molecule which binds to Cripto and a chemotherapeutic agent, e.g., an antimetabolite, thereby inhibiting growth of a tumor in the subject.

In one embodiment, the binding molecule and the chemotherapeutic agent, e.g., antimetabolite act, synergistically.

In one embodiment, the chemotherapeutic agent is an antimetabolite. In one embodiment, the antimetabolite is a pyrimidine analog. In one embodiment, the pyrimidine analog is 5′-fluorouracil.

In one embodiment, the binding molecule is an anti-Cripto antibody. In one embodiment, the binding molecule is a humanized anti-Cripto antibody. In one embodiment, the anti-Cripto antibody is conjugated to a maytansoid, e.g., DM4. In one embodiment, the maytansoid is conjugated to the antibody via a heterobifunctional crosslinking agent, e.g., SPDB. In one embodiment, an average of 3.5 molecules of DM4 is attached to the anti-cripto antibody.

In one embodiment, the binding molecule and the chemotherapeutic agent, e.g., antimetabolite, are administered in a single dose. In one embodiment, the binding molecule and the chemotherapeutic agent, e.g., antimetabolite, are administered biweekly. In one embodiment, the binding molecule and the chemotherapeutic agent, e.g., antimetabolite, are administered every three weeks.

In one embodiment, the effective dose of the binding molecule (e.g., humanized anti-Cripto antibody conjugated to a maytansinoid, e.g., B3F6.1-DM4) is selected from the group consisting of about 5 mg/kg, about 10 mg/kg, about 15 mg/kg, about 25 mg/kg and about 40 mg/kg. In a preferred embodiment, the effective dose of the binding molecule is 15 mg/kg.

In one embodiment, the binding molecule and the chemotherapeutic agent, e.g., antimetabolite, are administered intraperitoneally, orally, intranasally, subcutaneously, intramuscularly, topically, or intravenously.

In one embodiment, the subject is suffering from a cancer in an organ selected from the group consisting of brain, breast, testicular, colon, rectal, lung, ovary, bladder, uterine, cervical, pancreatic and stomach. In a preferred embodiment, the subject is suffering from colon cancer.

In yet another aspect, the invention provides a method of inhibiting growth of a tumor in a subject, comprising the steps of: (i) selecting a patient having an established tumor; and (ii) administering to the subject an effective dose of a binding molecule which binds to Cripto; thereby inhibiting growth of a tumor in the subject. In one embodiment, the binding molecule is an anti-Cripto antibody. In one embodiment, the binding molecule is a humanized anti-Cripto antibody. In one embodiment, the anti-Cripto antibody is conjugated to a maytansoid, e.g., DM4. In one embodiment, the maytansoid is conjugated to the antibody via a heterobifunctional crosslinking agent, e.g., SPDB. In one embodiment, an average of 3.5 molecules of DM4 is attached to the anti-cripto antibody.

In one embodiment, the binding molecule is administered in a single dose. In one embodiment, the binding molecule is administered biweekly. In one embodiment, the binding molecule is administered every three weeks.

In one embodiment, the effective dose of the binding molecule (e.g., humanized anti-Cripto antibody conjugated to a maytansinoid, e.g., B3F6.1-DM4) is selected from the group consisting of about 5 mg/kg, about 10 mg/kg, about 15 mg/kg, about 25 mg/kg and about 40 mg/kg. In one embodiment, the effective dose of the binding molecule (e.g., humanized anti-Cripto antibody conjugated to a maytansinoid, e.g., B3F6.1-DM4) is at least about 15 mg/kg. In one embodiment, the effective dose of the binding molecule (e.g., humanized anti-Cripto antibody conjugated to a maytansinoid, e.g., B3F6.1-DM4) is at least about 25 mg/kg. In one embodiment, the effective dose of the binding molecule (e.g., humanized anti-Cripto antibody conjugated to a maytansinoid, e.g., B3F6.1-DM4) is at least about 40 mg/kg).

In one embodiment, the binding molecule is administered intraperitoneally, orally, intranasally, subcutaneously, intramuscularly, topically, or intravenously.

In one embodiment, the subject is suffering from a cancer in an organ selected from the group consisting of brain, breast, testicular, colon, rectum, lung, ovary, bladder, uterine, cervical, pancreatic and stomach. In a preferred embodiment, the subject is suffering from colon cancer.

In yet another aspect, the invention provides a method of inhibiting growth of a tumor in a subject, comprising administering to the subject a single effective dose of a binding molecule which binds to Cripto, thereby inhibiting growth of a tumor in a subject.

In one embodiment, the binding molecule is an anti-Cripto antibody. In one embodiment, the binding molecule is a humanized anti-Cripto antibody. In one embodiment, the anti-Cripto antibody is conjugated to a maytansoid. In a preferred embodiment, the maytansinoid is DM4. In a preferred embodiment, an average of 3.5 molecules of DM4 is attached to one molecule of the antibody. In one embodiment, the maytansoid is conjugated to the antibody via a heterobifunctional crosslinking agent. In one embodiment, the heterobifunctional crosslinking agent is 4-(2-pyridyldithio)butanoic acid N-hydroxysuccinimide ester (SPDB).

In one embodiment, the effective single dose is selected from the group consisting of about 5 mg/kg, about 10 mg/kg, about 15 mg/kg, about 25 mg/kg and about 40 mg/kg.

In one embodiment, the subject is suffering from a cancer in an organ selected from the group consisting of brain, breast, testicular, colon, rectal, lung, ovary, bladder, uterine, cervical, pancreatic and stomach.

In another aspect, the invention provides a liquid aqueous pharmaceutical formulation comprising: (a) a therapeutically effective amount of binding molecule that binds to Cripto, (b) 10 mM sodium succinate with a pH of 5.0, (c) 120 mM L-glycine, (d) 120 mM glycerol, and (e) 0.01% Polysorbate 80.

In one embodiment, the binding molecule is a humanized anti-Cripto antibody. In one embodiment, the humanized anti-Cripto antibody is conjugated to a maytansoid. In one embodiment, the maytansinoid is DM4. In a preferred embodiment, an average of 3.5 molecules of DM4 attached to one molecule of the antibody. In one embodiment, the maytansoid is conjugated to the antibody via a heterobifunctional crosslinking agent. In one embodiment, the heterobifunctional crosslinking agent is 4-(2-pyridyldithio)butanoic acid N-hydroxysuccinimide ester (SPDB). In a preferred embodiment, the concentration of the binding molecule (e.g., humanized anti-Cripto antibody conjugated to DM4) is 5 mg/ml.

### BRIEF DESCRIPTION OF THE DRAWINGS

FIG. 1 shows the effect of a single dose (25 and 40 mg/kg/inj) or two doses (25 and 40 mg/kg/inj) of B3F6.1-DM4 dosed IV on various regimens on change in tumor weight in athymic nude mice bearing established CT-3 xenograft tumors.

FIG. 2 shows the effect of a single dose (15 mg/kg/inj) of B3F6.1-DM4, a single dose (30 mg/kg/inj) of 5-fluorouracil, and a combination of a single dose (15 mg/kg/inj) of B3F6.1-DM4 together with a single dose (30 mg/kg/inj) of 5-fluorouracil, each dosed IV, on change in tumor weight in athymic nude mice bearing established CT-3 xenograft tumors.

FIG. 3 shows the effect of a single dose (15 and 25 mg/kg/inj) of B3F6.1-DM4 dosed IV on change in tumor weight in athymic nude mice bearing large CT-3 xenograft tumors, e.g., tumors having a mean tumor weight of 550-775 mg.

FIG. 4 shows the effect of a single dose (5, 10 and 15 mg/kg/inj) of B3F6.1-SMCC-DM1 or a single dose (5, 10 and 15 mg/kg/inj) of B3F6.1-SPDB-DM4 dosed IV on change in tumor weight in athymic nude mice bearing established human testicular xenograft tumors.

### DETAILED DESCRIPTION OF THE INVENTION

The invention is based, at least in part, on the discovery that a humanized anti-Cripto antibody, B3F6.1, conjugated to a maytansoid (B3F6.1-DM4) is effective in inhibiting tumor cell growth in vivo in an animal model when administered in a single dose or in a biweekly dosage regimen. The biweekly dosing in the murine model is equivalent to a dose of once per every three weeks in primates, indicating that an effective dose of B3F6.1-DM4 in man includes a dosing regimen of administration once every 3 weeks. The invention is further based on the discovery that a single dose of B3F6.1-DM4 is effective in inhibiting growth of established tumors in an in vivo murine model. The invention is still further based on the discovery that the administration of B3F6.1-DM4 together with an additional agent, e.g., a chemotherapeutic agent, such as an antimetabolite, e.g., 5′-fluorouracil, results in a synergistic inhibition of tumor cell growth in vivo in an in vivo murine model.

Accordingly, the invention provides methods of inhibiting the growth of a tumor in a subject, comprising administering to the patient an effective dosage of a binding molecule which binds to Cripto, for example, a humanized anti-Cripto antibody conjugated to a maytansinoid (e.g., B3F6.1-DM4), wherein the binding molecule is administered once every three weeks. The invention further provides a method of inhibiting growth of a tumor in a subject, comprising administering to the subject an effective dosage of a binding molecule which binds to Cripto, e.g., a humanized anti-Cripto antibody conjugated to a maytansinoid (e.g., B3F6.1-DM4), and an additional chemotherapeutic agent, e.g, an antimetabolite, e.g., a pyrimidine analog, e.g., 5′-fluorouracil, thereby inhibiting growth of a tumor in the subject. The invention also provides a method of inhibiting growth of a tumor in a subject, comprising the steps of selecting a patient having an established tumor; and administering to the subject an effective dose of a binding molecule which binds to Cripto; thereby inhibiting growth of a tumor in the subject.

Before further description of the invention, for convenience, certain terms are described below:

### I. Definitions

The binding molecules of the invention are polypeptide molecules that comprise at least one binding domain which comprises a binding site that specifically binds to a human Cripto molecule. Exemplary sequences of human Cripto are shown in SEQ ID NO:6 (CR-1) and SEQ ID NO:7 (CR-3). CR-1 corresponds to the structural gene encoding the human Cripto protein expressed in the undifferentiated human teratocarcinoma cells and CR-3 corresponds to a complete copy of the mRNA containing seven base substitutions in the coding region representing both silent and replacement substitutions. CR-1 maps to chromosome 3, and CR-3 maps to Xq21-q22. Dono et al. 1991. Am J Hum Genet. 1991 49:555.

Preferably, the binding molecules of the invention comprise at least one CDR (e.g., 1, 2, 3, 4, 5, or preferably 6 CDRs) derived from the murine B3F6 antibody. The murine B3F6 antibody binds to an epitope in the domain spanning amino acid residues 46-62 of Cripto. The hybridoma that makes the murine B3F6 antibody (also referred to B3F6.17) was deposited with the ATCC under ACCESSION NO. PTA-3319). The antibody was made by immunizing mice with a Cripto fusion protein expressed in CHO cells. The fusion protein used for immunization comprised amino acid residues 1 to 169 of Cripto [amino acids 1-169 of SEQ ID NO: 6], fused to a human IgG₁ Fc domain (the construct is referred to as CR(del C)-Fc). The methods for making the B3F6 antibody are described in more detail, e.g., in WO 02/088170. In particular, exemplary humanized B3F6 antibodies can be found in WO 06/74397. A CHO cell producing one humanized version of the B3F6 antibody was deposited with the ATCC under ACCESSION NO. PTA-7284).

As used herein, an “established tumor” is a solid tumor of sufficient size such that nutrients, i.e., oxygen can no longer permeate to the center of the tumor from the subject's vasculature by osmosis and therefore the tumor requires its own vascular supply to receive nutrients.

In one embodiment, the subject methods are used to treat a vascularized tumor. A vascularized tumor includes tumors having the hallmarks of established vasculature. Such tumors are identified by their size and/or by the presence of markers of vessels or angiogenesis.

In one embodiment of the invention, a combination therapy is used to treat an established tumor, e.g., tumors of sufficient size such that nutrients can no longer permeate to the center of the tumor from the subject's vasculature by osmosis and therefore the tumor requires its own vascular supply to receive nutrients, i.e, a vascularized tumor. In one embodiment, a combination therapy is used to treat a tumor having dimensions of at least about 1 mm×1 mm. In another embodiment of the invention, a combination therapy is used to treat a tumor that is at least about 2 mm×2 mm. In yet another embodiment of the invention, a combination therapy is used to treat a tumor that is at least about 5 mm×5 mm. In other embodiments of the invention the tumor has a volume of at least about 1 cm³. In one embodiment, a combination therapy of the invention is used to treat a tumor that is large enough to be found by palpation or by imaging techniques well known in the art, such as MRI, ultrasound, or CAT scan.

As used herein the term “derived from” a designated protein refers to the origin of the polypeptide. In one embodiment, the polypeptide or amino acid sequence which is derived from a particular starting polypeptide is a CDR sequence or sequence related thereto. In one embodiment, the amino acid sequence which is derived from a particular starting polypeptide is not contiguous. For example, in one embodiment, one, two, three, four, five, or six CDRs are derived from a starting antibody. In one embodiment, the polypeptide or amino acid sequence which is derived from a particular starting polypeptide or amino acid sequence has an amino acid sequence that is essentially identical to that of the starting sequence, or a portion thereof wherein the portion consists of at least of at least 3-5 amino acids, 5-10 amino acids, at least 10-20 amino acids, at least 20-30 amino acids, or at least 30-50 amino acids, or which is otherwise identifiable to one of ordinary skill in the art as having its origin in the starting sequence. In one embodiment, the one or more CDR sequences derived from the starting antibody are altered to produce variant CDR sequences, wherein the variant CDR sequences maintain Cripto binding activity.

It will also be understood by one of ordinary skill in the art that the binding molecules of the invention may be modified such that they vary in amino acid sequence from the B3F6 molecule from which they were derived. For example, nucleotide or amino acid substitutions leading to conservative substitutions or changes at “non-essential” amino acid residues may be made (e.g., in CDR and/or framework residues). The binding molecules of the invention maintain the ability to bind to Cripto.

An isolated nucleic acid molecule encoding a non-natural variant of a polypeptide can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartie acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide may be replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence.

In one embodiment, the binding molecules comprise one binding site. In another embodiment, the binding molecules comprise at least two binding sites. In one embodiment, the binding molecules comprise two binding sites. In one embodiment, the binding molecules comprise three binding sites. In another embodiment, the binding molecules comprise four binding sites.

In one embodiment, the binding molecules of the invention are monomers. In another embodiment, the binding molecules of the invention are multimers. For example, in one embodiment, the binding molecules of the invention are dimers. In one embodiment, the dimers of the invention are homodimers, comprising two identical monomeric subunits. In another embodiment, the dimers of the invention are heterodimers, comprising two non-identical monomeric subunits. The subunits of the dimer may comprise one or more polypeptide chains. For example, in one embodiment, the dimers comprise at least two polypeptide chains. In one embodiment, the dimers comprise two polypeptide chains. In another embodiment, the dimers comprise four polypeptide chains (e.g., as in the case of antibody molecules).

Preferred binding molecules of the invention comprise framework and/or constant region amino acid sequences derived from a human amino acid sequence. For example, in one embodiment, a binding molecule of the invention is a chimeric antibody. In another embodiment, a binding molecule of the invention is a humanized antibody. However, binding polypeptides may comprise framework and/or constant region sequences derived from another mammalian species. For example, a primate framework region (e.g., non-human primate), heavy chain portion, and/or hinge portion may be included in the subject binding molecules. In one embodiment, one or more murine amino acids may be present in the framework region of a binding polypeptide, e.g., a human or non-human primate framework amino acid sequence may comprise one or more amino acid back mutations in which the corresponding murine amino acid residue is present. Preferred binding molecules of the invention are less immunogenic than the starting B3F6 murine antibody.

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. In one embodiment, a polypeptide of the invention comprises a polypeptide chain comprising a CH1 domain, at least a portion of a hinge domain, and a CH2 domain. In another embodiment, a polypeptide of the invention comprises a polypeptide chain comprising a CH1 domain and a CH3 domain. In another embodiment, a polypeptide of the invention comprises a polypeptide chain comprising a CH1 domain, at least a portion of a hinge domain, and a CH3 domain. In another embodiment, a polypeptide of the invention comprises a polypeptide chain comprising a CH3 domain. In one embodiment, a polypeptide of the invention lacks at least a portion of a CH2 domain (e.g., all or part of a CH2 domain). In another embodiment, a polypeptide of the invention comprises a complete Ig heavy chain. As set forth above, it will be understood by one of ordinary skill in the art that these domains (e.g., the heavy chain portions) may be modified such that they vary in amino acid sequence from the naturally occurring immunoglobulin molecule.

In one embodiment, at least two of the polypeptide chains of a binding molecule of the invention comprise at least one heavy chain portion derived from an antibody or immunoglobulin molecule. In one embodiment, at least two heavy chain portions of a polypeptide of the invention are present on different polypeptide chains and interact, e.g., via at least one disulfide linkage (Form A) or via non-covalent interactions (Form B) to form a dimeric polypeptide, each monomer of the dimer comprising at least one heavy chain portion.

In one embodiment, the heavy chain portions of one polypeptide chain of a dimer are identical to those on a second polypeptide chain of the dimer. In one embodiment, the monomers (or half-mers) of a dimer of the invention are identical to each other. In another embodiment, they are not identical. For example, each monomer may comprise a different target binding site.

In one embodiment, a binding molecule of the invention is held together by covalent interactions, e.g., disulfide bonds and is dimeric. In one embodiment, a dimer of the invention is held together by one or more disulfide bonds. In another embodiment, a dimer of the invention is held together by one or more, preferably two disulfide bonds. In another embodiment, a dimer of the invention is held together by one or more, preferably three disulfide bonds. In another embodiment, a dimer of the invention is held together by one or more, preferably four disulfide bonds. In another embodiment, a dimer of the invention is held together by one or more, preferably five disulfide bonds. In another embodiment a dimer of the invention is held together by one or more, preferably six disulfide bonds. In another embodiment, a dimer of the invention is held together by one or more, preferably seven disulfide bonds. In another embodiment, a dimer of the invention is held together by one or more, preferably eight disulfide bonds. In another embodiment, a dimer of the invention is held together by one or more, preferably nine disulfide bonds. In another embodiment, a dimer of the invention is held together by one or more, preferably ten disulfide bonds. In a further embodiment, a dimer of the invention is not held together by disulfide bonds, but is held together, e.g., by non-covalent interactions.

The heavy chain portions of a polypeptide may be derived from different immunoglobulin molecules. For example, a heavy chain portion of a polypeptide may comprise a CH1 domain derived from an IgG1 molecule and a hinge region derived from an IgG3 molecule. In another example, a heavy chain portion may comprise a hinge region derived, in part, from an IgG1 molecule and, in part, from an IgG3 molecule. In another example, a heavy chain portion may comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a VL or CL domain.

In one embodiment a polypeptide of the invention comprises an amino acid sequence or one or more moieties not derived from an Ig molecule. Exemplary modifications are described in more detail below. For example, in one embodiment, a polypeptide of the invention may comprise a flexible linker sequence. In another embodiment, a polypeptide may be modified to add one or more functional moieties (e.g., PEG, a drug, a prodrug, and/or a detectable label).

A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship. Exemplary chimeric polypeptides include fusion proteins and the chimeric hinge connecting peptides of the invention.

In one embodiment, a binding polypeptide of the invention is a fusion protein. In one embodiment, a fusion protein of the invention is a chimeric molecule that comprises a binding domain (which comprises at least one binding site) and a dimerization domain (which comprises at least one heavy chain portion). The heavy chain portion may be from any immunoglobulin, such as IgG1, IgG2, IgG3, or IgG4 subtypes, IgA, IgE, IgD or IgM. In one embodiment, a fusion protein further comprises a synthetic connecting peptide.

In another embodiment of the invention, a binding molecule is an “antibody-fusion protein chimera.” Such molecules comprise a molecule which combines at least one binding domain of an antibody with at least one fusion protein. Preferably, the interface between the two polypeptides is a CH3 domain of an immunoglobulin molecule.

The term “heterologous” as applied to a polynucleotide or a polypeptide, means that the polynucleotide or polypeptide is derived from a genotypically distinct entity from that of the rest of the entity to which it is being compared. For instance, a heterologous polynucleotide or antigen may be derived from a different species, different cell type, or the same type of cell of distinct individuals.

The term “ligand binding domain” or “ligand binding portion” as used herein refers to any native receptor (e.g., cell surface receptor) or any region or derivative thereof retaining at least a qualitative ligand binding ability, and preferably the biological activity of a corresponding native receptor.

The term “receptor binding domain” or “receptor binding portion” as used herein refers to a native ligand or a region or derivative thereof retaining at least a qualitative receptor binding ability, and preferably the biological activity of a corresponding native ligand.

In one embodiment, the binding molecules of the invention are “antibody” or “immunoglobulin” molecules, e.g., naturally occurring antibody or immunoglobulin molecules (or an antigen binding fragment thereof) or genetically engineered antibody molecules that bind antigen in a manner similar to antibody molecules. As used herein, the term “immunoglobulin” includes a polypeptide having a combination of two heavy and two light chains whether or not it possesses any relevant specific immunoreactivity. “Antibodies” refers to such assemblies which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “immunoglobulin” comprises five distinct classes of antibody that can be distinguished biochemically. All five classes of antibodies are within the scope of the present invention, the following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

As used herein the term “variable region CDR amino acid residues” includes amino acids in a CDR or complementarity determining region as identified using sequence or structure based methods. As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

| | CDR Definitions | | |
|---|---|---|---|
| | Kabat¹ | Chothia² | MacCallum³ |
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein the term “variable region framework (FR) amino acid residues” refers to those amino acids in the framework region of an Ig chain. The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable region and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light claim variable region CDRs. Similarly, using the definition of CDRs by Chothia et al. or McCallum et al. the framework region boundaries are separated by the respective CDR termini as described above. In preferred embodiments the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system, Kabat E A et al. Sequences of Proteins of Immunological Interest. Bethesda, US Department of Health and Human Services, NIH. 1991). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998 161:4083).

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

As indicated above, the variable region allows the antibody to selectively recognize and specifically bind epitopes on antigens. That is, the V_{L} domain and V_{H} domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the V_{H} and V_{L} chains.

The term “fragment” refers to a part or portion of an antibody or antibody chain comprising fewer amino acid residues than an intact or complete antibody or antibody chain. The term “antigen-binding fragment” refers to a polypeptide fragment of an immunoglobulin or antibody that binds antigen or competes with intact antibody (i.e., with the intact antibody from which they were derived) for antigen binding (i.e., specific binding). As used herein, the term “antigen-binding fragment” of an antibody molecule includes antigen-binding fragments of antibodies, for example, an antibody light chain (VL), an antibody heavy chain (VH), a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means.

As used herein, the term “binding site” comprises a region of a polypeptide which is responsible for selectively binding to a target molecule of interest (e.g. an antigen, ligand, receptor, substrate or inhibitor). Binding domains comprise at least one binding site. Exemplary binding domains include an antibody variable domain, a receptor binding domain of a ligand, a ligand binding domain of a receptor or an enzymatic domain.

As used herein the term “valency” refers to the number of potential target binding sites in a polypeptide. Each target binding site specifically binds one target molecule or specific site on a target molecule. When a polypeptide comprises more than one target binding site, each target binding site may specifically bind the same or different molecules (e.g., may bind to different ligands or different antigens, or different epitopes on the same antigen). The subject binding molecules have at least one binding site specific for a human Cripto molecule.

The term “specificity” refers to the ability to specifically bind (e.g., immunoreact with) a given target. A polypeptide may be monospecific and contain one or more binding sites which specifically bind a target or a polypeptide may be multispecific and contain two or more binding sites which specifically bind the same or different targets.

In one embodiment, a binding molecule of the invention is specific for more than one target. For example, in one embodiment, a multispecific binding molecule of the invention binds to Cripto and a second molecule expressed on a tumor cell. Exemplary antibodies which comprise antigen binding sites that bind to antigens expressed on tumor cells are known in the art and one or more CDRs from such antibodies can be included in a binding molecule of the invention. Exemplary antibodies include: 2B8, Lym 1, Lym 2, LL2, Her2, B1, MB1, BH3, B4, B72.3, 5E8, and 5E10.

In one embodiment, a binding molecule of the invention comprises a connecting peptide. The connecting peptides of the invention are synthetic. As used herein the term “synthetic” with respect to polypeptides includes polypeptides which comprise an amino acid sequence that is not naturally occurring. For example, non-naturally occurring polypeptides which are modified forms of naturally occurring polypeptides (e.g., comprising a mutation such as an addition, substitution or deletion) or which comprise a first amino acid sequence (which may or may not be naturally occurring) that is linked in a linear sequence of amino acids to a second amino acid sequence (which may or may not be naturally occurring) to which it is not naturally linked in nature.

Connecting peptides of the invention connect two domains (e.g., a binding domain and a dimerization domain) of a binding molecule of the invention. For example, connecting peptides connect a heavy chain portion to a binding domain comprising a binding site. In one embodiment, a connecting peptide connects two heavy chain constant region domains, such as CH1 and CH2 domains; CH1 and CH3 domains; hinge and CH1 domains; hinge and CH3 domains; VH and hinge domains, or a CH3 domain and a non-immunoglobulin polypeptide) in a linear amino acid sequence of a polypeptide chain. Preferably, such connecting peptides provide flexibility to the binding molecule and facilitate dimerization via disulfide bonding. In one embodiment, the connecting peptides of the invention are used to replace one or more heavy chain domains (e.g., at least a portion of a constant region domain (e.g., at least a portion of a CH2 domain) and/or at least a portion of the hinge region (e.g., at least a portion of the lower hinge region domain) in a domain deleted construct). For example, in one embodiment, a VH domain is fused to a CH3 domain via a connecting peptide (the C-terminus of the connecting peptide is attached to the N-terminus of the CH3 domain and the N-terminus of the connecting peptide is attached to the C-terminus of the VH domain). In another embodiment, a VL domain is fused to a CH3 domain via a connecting peptide (the C-terminus of the connecting peptide is attached to the N-terminus of the CH3 domain and the N-terminus of the connecting peptide is attached to the C-terminus of the VL domain. In another embodiment, a CH1 domain is fused to a CH3 domain via a connecting peptide (the C-terminus of the connecting peptide is attached to the N-terminus of the CH3 domain and the N-terminus of the connecting peptide is attached to the C-terminus of the CH1 domain).

In one embodiment, a synthetic connecting peptide comprises a portion of a constant region domain. For example, in one embodiment, a connecting peptide that replaces a CH2 domain may comprise a portion of the CH2 domain.

In one embodiment, a connecting peptide comprises or consists of a gly-ser linker. As used herein, the term “gly-ser linker” refers to a peptide that consists of glycine and serine residues. An exemplary gly/ser linker comprises the amino acid sequence GGGSSGGGSG (SEQ ID NO:8). In one embodiment, a connecting peptide of the invention comprises at least a portion of an upper hinge region (e.g., derived from an IgG1, IgG3, or IgG4 molecule), at least a portion of a middle hinge region (e.g., derived from an IgG1, IgG3, or IgG4 molecule) and a series of gly/ser amino acid residues (e.g., a gly/ser linker such as GGGSSGGGSG (SEQ ID NO:8)). In one embodiment, the connecting peptide comprises a substitution of one or more amino acids as compared to naturally occurring IgG1 or IgG3 hinge regions. In another embodiment, a connecting peptide comprises an amino acid sequence such as described in WO 02/060955. Connecting peptides are described in more detail below.

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

In one embodiment, a binding molecule of the invention comprises an antibody binding site. For example, in one embodiment, a binding molecule of the invention is a full-length antibody molecule. In another embodiment, a binding molecule of the invention is a fragment of an antibody molecule. In another embodiment, binding molecule of the invention is a modified or synthetic antibody molecule.

Binding molecules of the invention can be made using techniques that are known in the art. In one embodiment, the polypeptides of the invention are antibody molecules that have been “recombinantly produced,” i.e., are produced using recombinant DNA technology. Exemplary techniques for making antibody molecules are discussed in more detail below.

In one embodiment, the polypeptides of the invention are modified antibodies. As used herein, the term “modified antibody” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen). In another embodiment, a binding molecule of the invention is a fusion protein comprising at least one heavy chain portion lacking a CH2 domain and comprising a binding domain of a polypeptide comprising the binding portion of one member of a receptor ligand pair.

In one embodiment, the term, “modified antibody” according to the present invention includes immunoglobulins, antibodies, or immunoreactive fragments or recombinants thereof, in which at least a fraction of one or more of the constant region domains has been deleted or otherwise altered so as to provide desired biochemical characteristics such as the ability to non-covalently dimerize, increased ability to localize at the site of a tumor, or reduced serum half-life when compared with a whole, unaltered antibody of approximately the same immunogenicity. In one embodiment, the polypeptides of the present invention are domain deleted antibodies which comprise a polypeptide chain similar to an immunoglobulin heavy chain, but which lack at least a portion of one or more heavy chain domains. More preferably, one entire domain of the constant region of the modified antibody will be deleted and even more preferably all or part of the CH2 domain will be deleted.

In preferred embodiments, a polypeptide of the invention will not elicit a deleterious immune response in a human.

In one embodiment, a binding molecule of the invention comprises a constant region, e.g., a heavy chain constant region, which is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Exemplary modifications include additions, deletions or substitutions of one or more amino acids in one or more domains.

As used herein, the term “malignancy” refers to a non-benign tumor or a cancer. As used herein, the term “cancer” includes a malignancy characterized by deregulated or uncontrolled cell growth. Exemplary cancers include: carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques). Preferably, the binding molecules of the invention are engineered, e.g., to express a connecting peptide of the invention.

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

As used herein, the phrase “subject that would benefit from administration of a binding molecule” includes subjects, such as mammalian subjects, that would benefit from administration of a binding molecule used, e.g., for detection of an antigen recognized by a binding molecule (e.g., for a diagnostic procedure) and/or from treatment with a binding molecule to reduce or eliminate the target recognized by the binding molecule. For example, in one embodiment, the subject may benefit from reduction or elimination of a soluble or particulate molecule from the circulation or serum (e.g., a toxin or pathogen) or from reduction or elimination of a population of cells expressing the target (e.g., tumor cells). As described in more detail herein, the binding molecule can be used in unconjugated form or can be conjugated, e.g., to a drug, prodrug, or an isotope.

### II. Humanization

In one embodiment, the binding molecules of the invention comprise or are derived from at least one humanized B3F6 antibody variable region, e.g., a light chain or heavy chain variable region.

The term “humanized antibody” refers to an antibody comprising at least one chain comprising variable region framework residues substantially from a human antibody chain (referred to as the “acceptor antibody”) and at least one complementarity determining region (“CDR”) substantially from a non-human antibody (referred to as the “donor antibody”), in this case an anti-Cripto antibody, e.g., B3F6. Preferably, the constant region(s), if present, are also substantially or entirely from a human immunoglobulin.

The murine B3F6 antibody is described in WO 2006 074397. The sequences of the light chain variable regions and heavy chain variable regions of the murine B3F6 antibody are provided in SEQ ID NO: 39 and SEQ ID NO: 40, respectively. The CDRs of murine B3F6 are set forth below in Table 1:

TABLE 1
B3F6 CDR Sequences (Kabat Definition)

| | | |
|---|---|---|
| CDR L1 | RSSQSIVHSNGNTYLE | SEQ ID NO: 9 |
| CDR L2 | KVSNRFS | SEQ ID NO: 10 |
| CDR L3 | FQGSHVPLT | SEQ ID NO: 11 |
| CDR H1 | SYWIH | SEQ ID NO: 12 |
| CDR H2 | ENDPSNGRTNYNEKFKN | SEQ ID NO: 13 |
| CDR H3 | GPNYFYSMDY | SEQ ID NO: 14 |

The variable light chain of the murine B3F6 antibody is a member of mouse subgroup Kappa 2, with a 92.9% identity in 113 aa overlap (the consensus sequence of mouse subgroup Kappa 2 is shown in SEq ID NO: 41). The variable heavy chain is a member of mouse subgroup 2B with a 80.5% identity in 128 aa overlap (the consensus sequence of mouse subgroup 2B is shown in SEQ ID NO:42). The variable light chain corresponds to human subgroup Kappa 2 with a 76.3% identity in 114 aa overlap (the consensus sequence for human subgroup Kappa 2 is shown in SEQ ID NO:43). The variable heavy chain corresponds to human subgroup 1 with a 65.1% identity in 129 aa overlap (the consensus sequence of human subgroup 1 is shown in SEQ ID NO:44).

In one embodiment, an antigen binding molecule of the invention comprises at least one heavy or light chain CDR of a B3F6 antibody molecule. In another embodiment, an antigen binding molecule of the invention comprises at least two CDRs a B3F6 antibody molecule. In another embodiment, an antigen binding molecule of the invention comprises at least three CDRs from a B3F6 antibody molecule. In another embodiment, an antigen binding molecule of the invention comprises at least four CDRs from a B3F6 antibody molecule. In another embodiment, an antigen binding molecule of the invention comprises at least five CDRs from a B3F6 antibody molecule. In another embodiment, an antigen binding molecule of the invention comprises at least six CDRs from a B3F6 antibody molecule. In one embodiment, the at least one CDR (or at least one CDR from the greater than one B3F6 CDRs that are present in the binding molecule) is modified to vary in sequence from the CDR of a naturally occurring B3F6 molecule, yet retains the ability to bind to B3F6.

Humanized antibodies can be produced using recombinant DNA technology, see for example, e.g., Queen et al., Proc. Natl. Acad. Sci. USA, (1989), 86:10029-10033; Jones et al., Nature, (1986), 321:522-25; Riechmann et al., Nature, (1988), 332:323-27; Verhoeyen et al., Science, (1988), 239:1534-36; Orlandi et al., Proc. Natl. Acad. Sci. USA, (1989), 86:3833-37; U.S. Pat. Nos. 5,225,539; 5,530,101; 5,585,089; 5,693,761; 5,693,762; 6,180,370.

For example, when a preferred nonhuman donor antibody has been selected for humanization, an appropriate human acceptor antibody may be obtained, e.g., from sequence databases of expressed human antibody genes, from germline Ig sequences or a consensus sequence of several human antibodies. The substitution of nonhuman CDRs into a human variable domain framework is most likely to result in retention of their correct spatial orientation if the human variable domain framework adopts the same or similar conformation to the nonhuman variable framework from which the CDRs originated. This is achieved by obtaining the human variable domains from human acceptor antibodies whose framework sequences exhibit a high degree of sequence identity with the nonhuman variable framework domains from which the CDRs were derived. The heavy and light chain variable framework regions can be derived from the same or different human antibody sequences. Preferably the human acceptor antibody retains the canonical and interface residues of the donor antibody. Additionally, the human acceptor antibody preferably has substantial similarity in the length of CDR loops. See Kettleborough et al., Protein Engineering 4:773 (1991); Kolbinger et al., Protein Engineering 6:971 (1993) and Carter et al., WO 92/22653.

Having identified the CDRs of the donor antibody and appropriate human acceptor antibody, the next step is to determine which, if any, residues from these components should be substituted to optimize the properties of the resulting humanized antibody. Typically, some or all of the amino acids of the nonhuman, donor immunoglobulin light or heavy chain that are required for antigen binding (e.g., one or more CDRs) are used to substitute for the corresponding amino acids from the light or heavy chain of the human acceptor antibody. The human acceptor antibody retains some or all of the amino acids that are not required for antigen binding. In general, substitution of human amino acid residues with murine is minimized, because introduction of murine residues increases the risk of the antibody eliciting a human-anti-mouse-antibody (HAMA) response in humans. Art-recognized methods of determining immune response can be performed to monitor a HAMA response in a particular patient or during clinical trials. Patients administered humanized antibodies can be given an immunogenicity assessment at the beginning and throughout the administration of said therapy. The HAMA response is measured, for example, by detecting antibodies to the humanized therapeutic reagent, in serum samples from the patient using a method known to one in the art, including surface plasmon resonance technology (BIACORE) and/or solid-phase ELISA analysis.

When necessary, one or more residues in the human framework regions can be changed to residues at the corresponding positions in the murine antibody so as to preserve the binding affinity of the humanized antibody to the antigen. This change is sometimes called “back mutation.” Certain amino acids from the human variable region framework residues are selected for back mutation based on their possible influence on CDR conformation and/or binding to antigen. The placement of murine CDR regions with human variable framework region can result in conformational restraints, which, unless corrected by substitution of certain amino acid residues, lead to loss of binding affinity.

In one embodiment, the selection of amino acid residues for back mutation can determined, in part, by computer modeling, using art recognized techniques. In general, molecular models are produced starting from solved structures for immunoglobulin chains or domains thereof. The chains to be modeled are compared for amino acid sequence similarity with chains or domains of solved three-dimensional structures (e.g., X-ray structures) and the chains or domains showing the greatest sequence similarity is/are selected as starting points for construction of the molecular model. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

In another embodiment, a knowledge based approach or database analysis may be used for humanization. For example, such humanization strategy may be based on visual inspection and analysis of V region sequences according to the methods described in Rosok et al (Rosok M J, et al., 1996. J. Biol. Chem. 271: 22611-22618). Canonical determinants, surface residues, and potential contact residues are identified. Potential contact residues are noted and broadly classified according to the structural definition of CDR loops as defined by Chothia et al. (Chothia C and Lesk A M. 1987. J. Mol. Biol. 196: 901-917), sequence hypervariability as defined by Kabat et al. (Kabat E A, Wu T T, Reid-Miller M, Parry H M, and Gottesman K S. 1987. Sequences of Protein of Immunological Interest, U.S. department of Health and Human Services, NIH, Bethesda, Md.), and potential antigen contact residues as defined by MacCallum et al. (MacCallum R M, Martin A C R, and Thorton J M. 1996. J. Mol. Biol. 262: 732-745). Murine CDR loops, according to Kabat numbering and definition, are grafted in their entirety onto the acceptor human framework. Packing residues as defined by Padlan (Padlan E A. 1991. Mol Immunol. 28: 489-498) are identified and an attempt is made to conserve the packing residues in accordance with the strategy described in Singer et al. (Singer I I et al. 1993. J. Immunol. 150: 2844-2857). Each residue in the framework sequence is assigned a low, medium, or high “risk position” for antibody humanization as described in Harris and Bajorath (Harris L and Bajorath J. 1995. Protein Science 4: 306-310).

In general, low risk positions are kept human. For many of the nonidentical medium and high risk amino acid positions reference may be made to public or proprietary collections of humanized antibody sequences. In review of previously humanized antibody sequences, whether the inclusion of a human or murine (backmutation) amino acid residue resulted in functional binding activity was noted. In those cases where a substitution is considered, reference may be made to an amino acid substitution map (D. Bordo and P. Argos. 1991. J. Mol. Biol. 217: 721-729) to confirm the functional interchangeability of the residues.

The selection of amino acid residues for substitution can also be determined, in part, by examination of the characteristics of the amino acids at particular locations, or empirical observation of the effects of substitution or mutagenesis of particular amino acids. For example, when an amino acid differs between a nonhuman variable region framework residue and a selected human variable region framework residue, the human framework amino acid should usually be substituted by the equivalent framework amino acid from the nonhuman donor antibody when the amino acid from the donor antibody is a canonical residue, an interface packing residue, or an unusual or rare residue that is close to the binding site.

In one embodiment, a binding molecule of the invention further comprises at least one backmutation of a human amino acid residue to the corresponding mouse amino acid residue where the amino acid residue is an interface packing residue. “Interface packing residues” include those residues at the interface between VL and VH as defined, for example, by Novotny and Haber, Proc. Natl. Acad. Sci. USA, 82:4592-66 (1985).

In one embodiment, a binding molecule of the invention further comprises at least one backmutation of a human amino acid residue to the corresponding mouse amino acid residue is a canonical residue. “Canonical residues” are conserved framework residues within a canonical or structural class known to be important for CDR conformation (Tramontano et al., J. Mol. Biol. 215:175 (1990), all of which are incorporated herein by reference). Canonical residues include 2, 25, 27B, 28, 29, 30, 33, 48, 51, 52, 64, 71, 90, 94 and 95 of the light chain and residues 24, 26, 27 29, 34, 54, 55, 71 and 94 of the heavy chain. Additional residues (e.g., CDR structure-determining residues) can be identified according to the methodology of Martin and Thorton (1996) J. Mol. Biol. 263:800.

In one embodiment, a binding molecule of the invention further comprises at least one backmutation of a human amino acid residue to the corresponding mouse amino acid residue where the amino acid residue is at a position capable of interacting with a CDR. Notably, the amino acids at positions 2, 48, 64 and 71 of the light chain and 26-30, 71 and 94 of the heavy chain (numbering according to Kabat) are known to be capable of interacting with the CDRs in many antibodies. The amino acids at positions 35 in the light chain and 93 and 103 in the heavy chain are also likely to interact with the CDRs.

Exemplary techniques for selection of framework residues for substitution are set forth, for example, in U.S. Pat. No. 5,585,089. In that patent several categories of human framework amino acids which may be altered are described. In one embodiment, a category 2 amino acid is backmutated to the corresponding murine residue. Specifically, category 2 amino acids are amino acids in the framework of the human acceptor immunoglobulin which are unusual (i.e., “rare”, which as used herein indicates an amino acid occurring at that position in less than about 20% but usually less than about 10% of human heavy (respectively light) chain V region sequences in a representative data bank), and if the donor amino acid at that position is typical for human sequences (i.e., “common”, which as used herein indicates an amino acid occurring in more than about 25% but usually more than about 50% of sequences in a representative data bank), then the non-human donor amino acid (e.g., murine amino acid) rather than the human acceptor amino acid may be selected. This criterion helps ensure that an atypical amino acid in the human framework does not disrupt the antibody structure. Moreover, by replacing an unusual amino acid with an amino acid from the donor antibody that happens to be typical for human antibodies, the humanized antibody may be made less immunogenic.

All human light and heavy chain variable region sequences are respectively grouped into “subgroups” of sequences that are especially homologous to each other and have the same amino acids at certain critical positions (Kabat et al., op. cit.). When deciding whether an amino acid in a human acceptor sequence is “rare” or “common” among human sequences, it will often be preferable to consider only those human sequences in the same subgroup as the acceptor sequence.

In one embodiment, a category 3 amino acid is backmutated to the corresponding murine residue. Residues in category 3 are adjacent to one or more of the 3 CDR's in the primary sequence of the humanized immunoglobulin chain, the donor amino acid(s) rather than acceptor amino acid may be selected. These amino acids are particularly likely to interact with the amino acids in the CDR's and, if chosen from the acceptor, to distort the donor CDR's and reduce affinity. Moreover, the adjacent amino acids may interact directly with the antigen (Amit et al., Science, 233, 747-753 (1986)) and selecting these amino acids from the donor may be desirable to keep all the antigen contacts that provide affinity in the original antibody.

In one embodiment, a category 4 amino acid is backmutated to the corresponding murine residue. Category 4 amino acids are those which in 3-dimensional model, typically of the original donor antibody, shows that certain amino acids outside of the CDR's are close to the CDR's and have a good probability of interacting with amino acids in the CDR's by hydrogen bonding, Van der Waals forces, hydrophobic interactions, etc. At those amino acid positions, the donor immunoglobulin amino acid rather than the acceptor immunoglobulin amino acid may be selected. Amino acids according to this criterion will generally have a side chain atom within about 3 angstrom units of some atom in the CDR's and must contain an atom that could interact with the CDR atoms according to established chemical forces, such as those listed above.

In the case of atoms that may form a hydrogen bond, the 3 angstroms is measured between their nuclei, but for atoms that do not form a bond, the 3 angstroms is measured between their Van der Waals surfaces. Hence, in the latter case, the nuclei must be within about 6 angstroms (3+sum of the Van der Waals radii) for the atoms to be considered capable of interacting. In many cases the nuclei will be from 4 or 5 to 6 Å apart. In determining whether an amino acid can interact with the CDRs, it is preferred not to consider the last 8 amino acids of heavy chain CDR 2 as part of the CDRs, because from the viewpoint of structure, these 8 amino acids behave more as part of the framework.

Amino acids in the framework that are capable of interacting with amino acids in the CDR's, and which therefore belong to Category 4, may be distinguished in another way. The solvent accessible surface area of each framework amino acid is calculated in two ways: (1) in the intact antibody, and (2) in a hypothetical molecule consisting of the antibody with its CDRs removed. A significant difference between these numbers of about 10 square angstroms or more shows that access of the framework amino acid to solvent is at least partly blocked by the CDRs, and therefore that the amino acid is making contact with the CDRs. Solvent accessible surface area of an amino acid may be calculated based on a 3-dimensional model of an antibody, using algorithms known in the art (e.g., Connolly, J. Appl. Cryst. 16, 548 (1983) and Lee and Richards, J. Mol. Biol. 55, 379 (1971)). Framework amino acids may also occasionally interact with the CDR's indirectly, by affecting the conformation of another framework amino acid that in turn contacts the CDR's.

The amino acids at several positions in the framework are known to be capable of interacting with the CDRs in many antibodies (Chothia and Lesk, J. Mol. Biol. 196, 901 (1987), Chothia et al., Nature 342, 877 (1989), and Tramontano et al., J. Mol. Biol. 215, 175 (1990), all of which are incorporated herein by reference), notably at positions 2, 48, 64 and 71 of the light chain and 26-30, 71 and 94 of the heavy chain (numbering according to Kabat, op. cit.), and therefore these amino acids will generally be in Category 4. In one embodiment, humanized immunoglobulins of the present invention will include donor amino acids (where different) in category 4 in addition to these. The amino acids at positions 35 in the light chain and 93 and 103 in the heavy chain are also likely to interact with the CDRs. Accordingly, in one embodiment, one or more donor amino acid rather than the acceptor amino acid (when they differ) may be included in a humanized immunoglobulin. On the other hand, certain positions that may be in Category 4 such as the first 5 amino acids of the light chain may sometimes be chosen from the acceptor immunoglobulin without loss of affinity in the humanized immunoglobulin.

In addition to the above categories which describe when an amino acid in the humanized immunoglobulin may be taken from the donor, certain amino acids in the humanized immunoglobulin may be taken from neither the donor nor acceptor, if they fall into Category 5. If the amino acid at a given position in the donor immunoglobulin is “rare” for human sequences, and the amino acid at that position in the acceptor immunoglobulin is also “rare” for human sequences, as defined above, then the amino acid at that position in the humanized immunoglobulin may be chosen to be some amino acid “typical” of human sequences. A preferred choice is the amino acid that occurs most often at that position in the known human sequences belonging to the same subgroup as the acceptor sequence.

In one embodiment, a binding molecule of the invention comprises three B3F6 light chain CDRs (CDRL1, CDRL2, and CDRL3) and a human light chain framework region. In one embodiment, the most suitable expressed human light chain framework is human gi-21669417 (BAC01733) (SEQ ID NO: 45). In one embodiment, a binding molecule of the invention further comprises a least one backmutation of a human amino acid residue to the corresponding mouse amino acid residue at at least one position selected from the group consisting of: 2 and 100. In one embodiment, a binding molecule of the invention further comprises one backmutation of a human amino acid residue to the corresponding mouse amino acid residue at one position selected from the group consisting of: 2 and 100. In another embodiment the binding molecule comprises backmutations at positions 2 and 100 of the humanized B3F6 light chain. In another embodiment, a binding molecule of the invention comprises a backmutation at position 2 of the humanized B3F6 light chain and at least one additional backmutation. In another embodiment, a binding molecule of the invention comprises a backmutation at position 100 of the humanized B3F6 light chain and at least one additional backmutation.

In one embodiment, a binding molecule of the invention comprises three B3F6 heavy chain CDRs (CDRH1, CDRH2, and CDRH3) and a human heavy chain framework region. In one embodiment of the invention, the most suitable expressed human heavy chain framework is gi-14289106 (AAK57792) (SEQ ID NO: 46). In one embodiment, a binding molecule of the invention comprises a least one backmutation of a human amino acid residue to the corresponding mouse amino acid residue at at least one position selected from the group consisting of: 1, 48, 67, 71, 73, 81, 82b, 93, and 112. In one embodiment, a binding molecule of the invention comprises one backmutation of a human amino acid residue to the corresponding mouse amino acid residue at one position selected from the group consisting of: 1, 48, 67, 71, 73, 81, 82b, 93, and 112. In one embodiment, a binding molecule of the invention comprises two backmutations of a human amino acid residue to the corresponding mouse amino acid residue at two positions selected from the group consisting of: 1, 48, 67, 71, 73, 81, 82b, 93, and 112. In one embodiment, a binding molecule of the invention comprises three backmutations of a human amino acid residue to the corresponding mouse amino acid residue at three positions selected from the group consisting of: 1, 48, 67, 71, 73, 81, 82b, 93, and 112. In one embodiment, a binding molecule of the invention comprises four backmutations of a human amino acid residue to the corresponding mouse amino acid residue at four positions selected from the group consisting of: 1, 48, 67, 71, 73, 81, 82b, 93, and 112. In one embodiment, a binding molecule of the invention comprises five backmutations of a human amino acid residue to the corresponding mouse amino acid residue at five positions selected from the group consisting of: 1, 48, 67, 71, 73, 81, 82b, 93, and 112. In one embodiment, a binding molecule of the invention comprises six backmutations of a human amino acid residue to the corresponding mouse amino acid residue at six three positions selected from the group consisting of: 1, 48, 67, 71, 73, 81, 82b, 93, and 112. In one embodiment, a binding molecule of the invention comprises seven backmutations of a human amino acid residue to the corresponding mouse amino acid residue at seven positions selected from the group consisting of: 1, 48, 67, 71, 73, 81, 82b, 93, and 112. In one embodiment, a binding molecule of the invention further comprises backmutations of a human amino acid residue to the corresponding mouse amino acid residue at eight positions selected from the group consisting of: 1, 48, 67, 71, 73, 81, 82b, 93, and 112. In one embodiment, a binding molecule of the invention comprises nine backmutations of a human amino acid residue to the corresponding mouse amino acid residue at nine positions selected from the group consisting of: 1, 48, 67, 71, 73, 81, 82b, 93, and 112.

In one embodiment, the invention pertains to humanized variable regions of the B3F6 antibody and polypeptides comprising such humanized variable regions.

In one embodiment, a binding molecule of the invention comprises a CDR grafted light chain variable region sequence shown in amino acids 1-112 of SEQ ID NO:52. In one embodiment, a binding molecule of the invention comprises a CDR grafted light chain variable region sequence shown in amino acids 1-121 of SEQ ID NO:55.

In one embodiment, a binding molecule of the invention comprises a light chain version 1 variable region sequence shown in SEQ ID NO:47. In one embodiment, a binding molecule of the invention comprises a heavy chain version 1 variable region sequence shown in SEQ ID NO:48. In one embodiment, a binding molecule of the invention comprises a heavy chain version 2 variable region sequence shown in SEQ ID NO:49.

In another embodiment, a binding molecule of the invention comprises a light chain version 2 variable region sequence shown in SEQ ID NO:50. In one embodiment, a binding molecule of the invention comprises a heavy chain version 3 variable region sequence shown in SEQ ID NO:51.

In one embodiment, a binding molecule of the invention comprises a CDR grafted light chain shown in SEQ ID NO:52. In one embodiment, a binding molecule of the invention comprises a version 1 light chain shown in SEQ ID NO:53. In one embodiment, a binding molecule of the invention comprises a version 2 light chain shown in SEQ ID NO:54.

In one embodiment, a binding molecule of the invention comprises a CDR grafted heavy chain shown in SEQ ID NO:55. In one embodiment, a binding molecule of the invention comprises a version 1 heavy chain shown in SEQ ID NO:56. In one embodiment, a binding molecule of the invention comprises a version 2 heavy chain shown in SEQ ID NO:57. In one embodiment, a binding molecule of the invention comprises a version 3 heavy chain shown in SEQ ID NO:58.

In one embodiment, a binding molecule of the invention comprises a CDR grafted domain deleted heavy chain shown in SEQ ID NO:59. In one embodiment, a binding molecule of the invention comprises a version 1 domain deleted heavy chain shown in SEQ ID NO:60. In one embodiment, a binding molecule of the invention comprises a version 2 domain deleted heavy chain shown in SEQ ID NO:61. In one embodiment, a binding molecule of the invention comprises a version 3 domain deleted heavy chain shown in SEQ ID NO:62.

In one embodiment, a binding molecule of the invention comprises a CDR grafted light chain sequence shown in SEQ ID NO:63, which includes an optional signal sequence. In one embodiment, a binding molecule of the invention comprises a version 1 light chain sequence shown in SEQ ID NO:64, which includes an optional signal sequence. In one embodiment, a binding molecule of the invention comprises a version 2 light chain sequence shown in SEQ ID NO:65, which includes an optional signal sequence.

In one embodiment, a binding molecule of the invention comprises a CDR grafted heavy chain sequence shown in SEQ ID NO:66, which includes an optional signal sequence. In one embodiment, a binding molecule of the invention comprises a version 1 heavy chain sequence shown in SEQ ID NO:67, which includes an optional signal sequence. In one embodiment, a binding molecule of the invention comprises a version 2 heavy chain sequence shown in SEQ ID NO:68, which includes an optional signal sequence. In one embodiment, a binding molecule of the invention comprises a version 3 heavy chain sequence shown in SEQ ID NO:69, which includes an optional signal sequence.

In one embodiment, a light chain comprising murine B3F6 CDRs and human framework regions is combined with a heavy chain comprising murine B3F6 CDRs and human framework regions. In one embodiment, a light chain comprising murine B3F6 CDRs and human framework regions is combined with a humanized version of a B3F6 heavy chain comprising at least one backmutation of a human framework amino acid residue to the corresponding murine amino acid residue. In another embodiment, a humanized version of a B3F6 light chain comprising at least one backmutation of a human framework amino acid residue to the corresponding murine amino acid residue is combined with a humanized version of a B3F6 heavy chain comprising at least one backmutation of a human framework amino acid residue to the corresponding murine amino acid residue. In another embodiment a light chain comprising murine B3F6 CDRs and human framework regions and at least one backmutation of a human framework amino acid residue to the corresponding murine amino acid residue is combined with a humanized version of a B3F6 heavy chain. Exemplary combinations are described in more detail in the examples of WO 2006 074397. For example, in one embodiment the humanized L1 light chain of the examples of WO 2006 074397 is combined with the H1 heavy chain of the examples of WO 2006 074397 to make the version 1 humanized B3F6 antibody. In another embodiment the humanized L1 light chain of the examples of WO 2006 074397 is combined with the H2 heavy chain of the examples of WO 2006 074397 to make the version 2 humanized B3F6 antibody. This version of humanized B3F6 is produced by the CHO cell line deposited with the ATCC under ACCESSION No. PTA-7284. In another embodiment, the humanized L1 light chain of the examples of WO 2006 074397 is combined with the H3 heavy chain of the examples of WO 2006 074397 to make the version 3 humanized B3F6 antibody. In another embodiment the humanized L2 light chain of the examples of WO 2006 074397 is combined with the H1 heavy chain of the examples of WO 2006 074397 to make the version 4 humanized B3F6 antibody. In another embodiment the humanized L2 light chain of the examples of WO 2006 074397 is combined with the H2 heavy chain of the examples of WO 2006 074397 to make the version 5 humanized B3F6 antibody. In another embodiment the humanized L2 light chain of the examples of WO 2006 074397 is combined with the H3 heavy chain of the examples of WO 2006 074397 to make the version 6 humanized B3F6 antibody. It will be apparent to one of ordinary skill in the art that such combinations are within the scope of this invention.

In one embodiment, a binding molecule of the invention is the humanized antibody made by the cell line deposited with the American Type Culture Collection (ATCC) 10801 University Boulevard, Manassas, Va., 20110 under ATCC ACCESSION NUMBER PTA-7284 under conditions of the Budapest treaty.

### II. Forms of Binding Molecules

A. Antibodies or Portions Thereof

In one embodiment, a binding molecule of the invention is an antibody molecule. For example, in one embodiment a binding molecule of the invention is a humanized antibody or portion thereof that binds to Cripto. In another embodiment, a binding molecule of the invention is multivalent and comprises an antigen binding fragment of a humanized antibody that binds to Cripto and a second antigen binding fragment of an antibody.

In one embodiment, other anti-Cripto antibodies may be made. In addition, binding sites for incorporation into multivalent anti-Cripto antibodies may be made. For example, antibodies are preferably raised in mammals by multiple subcutaneous or intraperitoneal injections of the relevant antigen (e.g., purified tumor associated antigens or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature, 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

Those skilled in the art will also appreciate that DNA encoding antibodies or antibody fragments (e.g., antigen binding sites) may also be derived from antibody phage libraries, e.g., using pd phage or Fd phagemid technology. Exemplary methods are set forth, for example, in EP 368 684 B1; U.S. Pat. No. 5,969,108, Hoogenboom, H. R. and Chames. 2000. Immunol. Today 21:371; Nagy et al. 2002. Nat. Med. 8:801; Huie et al. 2001. Proc. Natl. Acad. Sci. USA 98:2682; Lui et al. 2002. J. Mol. Biol. 315:1063, each of which is incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al. 2000. Nat. Biotechnol. 18:1287; Wilson et al. 2001. Proc. Natl. Acad. Sci. USA 98:3750; or Irving et al. 2001 J. Immunol. Methods 248:31. In yet another embodiment, cell surface libraries can be screened for antibodies (Boder et al. 2000. Proc. Natl. Acad. Sci. USA 97:10701; Daugherty et al. 2000 J. Immunol. Methods 243:211. Such procedures provide alternatives to traditional hybridoma techniques for the isolation and subsequent cloning of monoclonal antibodies.

In another embodiment of the present invention a binding site of a binding molecule of the invention may be provided by a human or substantially human antibody. Human or substantially human antibodies may be made in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Moreover, genetic sequences useful for producing the binding molecules of the present invention may be obtained from a number of different sources. For example, as discussed extensively above, a variety of human antibody genes are available in the form of publicly accessible deposits. Many sequences of antibodies and antibody-encoding genes have been published and suitable antibody genes can be chemically synthesized from these sequences using art recognized techniques. Oligonucleotide synthesis techniques compatible with this aspect of the invention are well known to the skilled artisan and may be carried out using any of several commercially available automated synthesizers. In addition, DNA sequences encoding several types of heavy and light chains set forth herein can be obtained through the services of commercial DNA synthesis vendors. The genetic material obtained using any of the foregoing methods may then be altered or synthetic to provide obtain polypeptides of the present invention.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

It will further be appreciated that the scope of this invention further encompasses alleles, variants and mutations of antigen binding DNA sequences.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis. Exemplary antibodies or fragments thereof for use in the binding molecules of the invention include antibodies that recognize the targets set forth herein.

In certain embodiments, antigen binding fragments of antibodies can be produced using techniques well known in the art.

B. Modified Antibodies

In one embodiment, a binding molecule of the invention comprises or consists of a modified antibody, i.e., and molecule that is derived from an antibody, but is not a wild-type antibody, e.g., minibodies (minibodies can be made using methods described in the art (see, e.g., see e.g., U.S. Pat. No. 5,837,821 or WO 94/09817A1)). etc.

1. Domain Deleted Antibodies

In one embodiment, a binding molecule of the invention comprises synthetic constant regions wherein one or more domains are partially or entirely deleted (“domain-deleted antibodies”). In especially preferred embodiments compatible modified antibodies will comprise domain deleted constructs or variants wherein the entire CH2 domain has been removed (ΔCH2 constructs). For other preferred embodiments a short connecting peptide may be substituted for the deleted domain to provide flexibility and freedom of movement for the variable region. Those skilled in the art will appreciate that such constructs are particularly preferred due to the regulatory properties of the CH2 domain on the catabolic rate of the antibody.

In another embodiment, the modified antibodies of the invention are CH2 domain deleted antibodies. Domain deleted constructs can be derived using a vector (e.g., from IDEC Pharmaceuticals, San Diego) encoding an IgG₁ human constant domain (see, e.g., WO 02/060955A2 and WO02/096948A2). This exemplary vector was engineered to delete the CH2 domain and provide a synthetic vector expressing a domain deleted IgG₁ constant region. Genes encoding the murine variable region of the C2B8 antibody, 5E8 antibody, B3F6 antibody, or the variable region of the humanized CC49 antibody have been then inserted in the synthetic vector and cloned. When expressed in transformed cells, these vectors provided C2B8.ΔCH2, 5E8.ΔCH2, B3F6.ΔCH2 or huCC49.ΔCH2 or respectively. These constructs exhibit a number of properties that make them particularly attractive candidates for monomeric subunits.

A CH2 domain-deleted chimeric B3F6 (chB3F6ΔCH2) antibody constructed using a hinge region connecting peptide G1/G3/Pro243Ala244Pro245+[Gly/Ser] (SEQ ID NO: 5) is described in WO 2006 074397. “chB3F6” is a chimeric anti-CRIPTO monoclonal antibody consisting of murine heavy and light chain variable domains fused to human heavy and light chain constant domains, respectively. The DNA sequence of heavy chain CH2 domain-deleted chimeric anti-CRIPTO monoclonal antibody consisting of murine heavy and light chain variable domains fused to human heavy and light chain constant domains, respectively (chB3F6) containing G1/G3/Pro243Ala244Pro245+[GlySer] hinge connecting peptide is shown in SEQ ID NO: 1. The DNA sequence of light chain CH2 domain-deleted chB3F6 is shown in SEQ ID NO: 2. The amino acid sequence of heavy chain CH2 domain-deleted chB3F6 containing G1/G3/Pro243Ala244Pro245+[GlySer] hinge connecting peptide is shown in SEQ ID NO: 3. The amino acid sequence of light chain CH2 domain-deleted chB3F6 is shown in SEQ ID NO: 4. The constant region sequence used to make domain deleted antibodies (comprising a hinge connecting peptide (HCP)) is shown in SEQ ID NO: 70 and the full length IgG1 constant region sequence used to make full-length antibodies is shown in SEQ ID NO: 71. Humanized domain deleted B3F6 antibodies have also been produced and are described in more detail in the examples of WO 2006 074397.

It will be noted that these exemplary constructs were engineered to fuse the CH3 domain directly to a hinge region of the respective polypeptides of the invention. In other constructs it may be desirable to provide a peptide spacer between the hinge region and the synthetic CH2 and/or CH3 domains. For example, compatible constructs could be expressed wherein the CH2 domain has been deleted and the remaining CH3 domain (synthetic or unsynthetic) is joined to the hinge region with a 5-20 amino acid spacer. Such a spacer may be added, for instance, to ensure that the regulatory elements of the constant domain remain free and accessible or that the hinge region remains flexible. For example, a domain deleted B3F6 construct having a short amino acid spacer GGSSGGGGSG (SEQ. ID No. 8) substituted for the CH2 domain and the lower hinge region (B3F6.ΔCH2 [gly/ser]) can be used. Other exemplary connecting peptides are shown in Table 2. These connecting peptides can be used with any of the polypeptides of the invention. Preferably, the connecting peptides are used with a polypeptide lacking a CH2 heavy chain domain. Preferably, any linker compatible with the instant invention will be relatively non-immunogenic and not inhibit the non-covalent association of the polypeptides of the invention.

In one embodiment, a polypeptide of the invention comprises an immunoglobulin heavy chain having deletion or substitution of a few or even a single amino acid as long as it permits the desired covalent or non-covalent association between the monomeric subunits. For example, the mutation of a single amino acid in selected areas of the CH2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other preferred embodiments may comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

It is known in the art that the constant region mediates several effector functions. For example, binding of the C1 component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In one embodiment, effector functions may be eliminated or reduced by using a constant region of an IgG4 antibody, which is thought to be unable to deplete target cells, or making Fc variants, wherein residues in the Fc region critical for effector function(s) are mutated using techniques known in the art, for example, U.S. Pat. No. 5,585,097. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate compliment binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. More generally, those skilled in the art will realize that antibodies modified as described herein may exert a number of subtle effects that may or may not be readily appreciated. However the resulting physiological profile, bioavailability and other biochemical effects of the modifications, such as tumor localization, biodistribution and serum half-life, may easily be measured and quantified using well know immunological techniques without undue experimentation.

In one embodiment, modified forms of antibodies can be made from a whole precursor or parent antibody using techniques known in the art. Exemplary techniques are discussed in more detail below

A polypeptide comprising a heavy chain portion may or may not comprise other amino acid sequences or moieties not derived from an immunoglobulin molecule. Such modifications are described in more detail below. For example, in one embodiment, a polypeptide of the invention may comprise a flexible linker sequence. In another embodiment, a polypeptide may be modified to add a functional moiety such as a drug or PEG.

2. Bispecific Binding Molecules

In one embodiment, a binding molecule of the invention is bispecific. For example, in one embodiment, a binding molecule binds to Cripto and another molecule. In one embodiment, a bispecific binding molecule of the present invention may comprise an additional binding site that binds to one or more tumor molecules or molecules associated with tumor cell growth. In one embodiment, for neoplastic disorders, an antigen binding site (i.e. the variable region or immunoreactive fragment or recombinant thereof) of the disclosed polypeptides binds to a selected tumor associated molecule at the site of the malignancy. Given the number of reported molecules associated with neoplasias tumor cell growth, and the number of related antibodies, those skilled in the art will appreciate that the binding sites of the claimed binding molecules may therefore be derived from any one of a number of whole antibodies. More generally, binding sites useful in the present invention may be obtained or derived from any antibody (including those previously reported in the literature) that reacts with a target or marker associated with the selected condition. Further, the parent or precursor antibody, or fragment thereof, used to generate the disclosed polypeptides may be murine, human, chimeric, humanized, non-human primate or primatized. In other preferred embodiments the polypeptides of the present invention may comprise single chain antibody constructs (such as that disclosed in U.S. Pat. No. 5,892,019 which is incorporated herein by reference) having altered constant domains as described herein. Consequently, any of these types of antibodies can be used to obtain a binding site that may be incorporated into a bispecific molecule of the invention.

As used herein, “tumor associated molecules” means any antigen or target molecule which is generally associated with tumor cells, i.e., being expressed at the same or to a greater extent as compared with normal cells. More generally, tumor associated molecules comprise any molecule that provides for the localization of immunoreactive antibodies at a neoplastic cell irrespective of its expression on non-malignant cells. Such molecules may be relatively tumor specific and limited in their expression to the surface of malignant cells. Alternatively, such molecules may be found on both malignant and non-malignant cells. For example, CD20 is a pan B antigen that is found on the surface of both malignant and non-malignant B cells that has proved to be an extremely effective target for immunotherapeutic antibodies for the treatment of non-Hodgkin's lymphoma.

In this respect, pan T cell antigens such as CD2, CD3, CD5, CD6 and CD7 also comprise tumor associated molecules within the meaning of the present invention. Still other exemplary tumor associated molecules comprise but not limited to Lewis Y, MAGE-1, MAGE-3, MUC-1, HPV 16, HPV E6 & E7, TAG-72, CEA, L6-Antigen, CD19, CD22, CD37, CD52, HLA-DR, EGF Receptor and HER2 Receptor. In many cases immunoreactive antibodies for each of these antigens have been reported in the literature. Those skilled in the art will appreciate that each of these antibodies may serve as a precursor for polypeptides of the invention in accordance with the present invention.

Previously reported antibodies that react with tumor associated molecules may be altered as described herein to provide one or more binding sites for a polypeptide of the present invention. Exemplary antibodies that may be used to provide binding sites for the subject polypeptides (or from which binding sites may be derived) include, but are not limited to 2B8 and C2B8 (Zevalin® and Rituxan®, IDEC Pharmaceuticals Corp., San Diego), Lym 1 and Lym 2 (Techniclone), LL2 (Immunomedics Corp., New Jersey), HER2 (Herceptin®, Genentech Inc., South San Francisco), B1 (Bexxar®, Coulter Pharm., San Francisco), Campath® (Millennium Pharmaceuticals, Cambridge) MB1, BH3, B4, B72.3 (Cytogen Corp.), CC49 (National Cancer Institute) and 5E10 (University of Iowa). In preferred embodiments, the polypeptides of the present invention will bind to the same tumor associated antigens as the antibodies enumerated immediately above. In particularly preferred embodiments, the polypeptides will be derived from or bind the same antigens as 2B8, C2B8, CC49 and C5E10 and, even more preferably, will comprise domain deleted antibodies (i.e., ΔCH2 antibodies).

In a first preferred embodiment, a bispecific molecule of the invention will bind to the same tumor associated antigen as Rituxan®. Rituxan® (also known as, rituximab, IDEC-C2B8 and C2B8) was the first FDA-approved monoclonal antibody for treatment of human B-cell lymphoma (see U.S. Pat. Nos. 5,843,439; 5,776,456 and 5,736,137 each of which is incorporated herein by reference). Y2B8 (90Y labeled 2B8; Zevalin®; ibritumomab tiuxetan) is the murine parent of C2B8. Rituxan® is a chimeric, anti-CD20 monoclonal antibody which is growth inhibitory and reportedly sensitizes certain lymphoma cell lines for apoptosis by chemotherapeutic agents in vitro. The antibody efficiently binds human complement, has strong FcR binding, and can effectively kill human lymphocytes in vitro via both complement dependent (CDC) and antibody-dependent (ADCC) mechanisms (Reff et al., Blood 83: 435-445 (1994)). Those skilled in the art will appreciate that bispecific binding molecules which bind to Cripto and CD20+ according to the instant disclosure, may be used in conjugated or unconjugated forms to effectively treat patients presenting with CD20+ malignancies. More generally, it must be reiterated that the polypeptides disclosed herein may be used in either a “naked” or unconjugated state or conjugated to a cytotoxic agent to effectively treat any one of a number of disorders.

In other preferred embodiments of the present invention, a bispecific polypeptide of the invention may comprise a binding site from the CC49 antibody (or derived from the CC49 antibody). As previously alluded to, CC49 binds human tumor associated antigen TAG-72 which is associated with the surface of certain tumor cells of human origin, specifically the LS 174T tumor cell line. LS 174T [American Type Culture Collection (herein ATCC) No. CL 188] is a variant of the LS180 (ATCC No. CL 187) colon adenocarcinoma line.

It will further be appreciated that numerous murine monoclonal antibodies have been developed which have binding specificity for TAG-72. One of these monoclonal antibodies, designated B72.3, is a murine IgG1 produced by hybridoma B72.3 (ATCC No. HB-8108). B72.3 is a first generation monoclonal antibody developed using a human breast carcinoma extract as the immunogen (see Colcher et al., Proc. Natl. Acad. Sci. (USA), 78:3199-3203 (1981); and U.S. Pat. Nos. 4,522,918 and 4,612,282 each of which is incorporated herein by reference). Other monoclonal antibodies directed against TAG-72 are designated “CC” (for colon cancer). As described by Schlom et al. (U.S. Pat. No. 5,512,443 which is incorporated herein by reference) CC monoclonal antibodies are a family of second generation murine monoclonal antibodies that were prepared using TAG-72 purified with B72.3. Because of their relatively good binding affinities to TAG-72, the following CC antibodies have been deposited at the ATCC, with restricted access having been requested: CC49 (ATCC No. HB 9459); CC 83 (ATCC No. HB 9453); CC46 (ATCC No. HB 9458); CC92 (ATTCC No. HB 9454); CC30 (ATCC No. HB 9457); CC11 (ATCC No. 9455); and CC15 (ATCC No. HB 9460). U.S. Pat. No. 5,512,443 further teaches that the disclosed antibodies may be altered into their chimeric form by substituting, e.g., human constant regions (Fc) domains for mouse constant regions by recombinant DNA techniques known in the art. Besides disclosing murine and chimeric anti-TAG-72 antibodies, Schlom et al. have also produced variants of a humanized CC49 antibody as disclosed in PCT/US99/25552 and single chain constructs as disclosed in U.S. Pat. No. 5,892,019 each of which is also incorporated herein by reference. Those skilled in the art will appreciate that each of the foregoing antibodies, constructs or recombinants, and variations thereof, may be synthetic and used in making a bispecific molecule of the invention.

In addition to the anti-TAG-72 antibodies discussed above, various groups have also reported the construction and partial characterization of domain-deleted CC49 and B72.3 antibodies (e.g., Calvo et al. Cancer Biotherapy, 8(1):95-109 (1993), Slavin-Chiorini et al. Int. J. Cancer 53:97-103 (1993) and Slavin-Chiorini et al. Cancer. Res. 55:5957-5967 (1995). Such constructs may also be included in a bispecific binding molecule of the invention.

In one embodiment, a bispecific binding molecule of the invention binds to CD23 (U.S. Pat. No. 6,011,138). In a preferred embodiment, a bispecific binding molecule of the invention comprises a binding site that binds to the same epitope as the 5E8 antibody. In another embodiment, a binding molecule of the invention comprises at least one CDR from an anti-CD23 antibody, e.g., the 5E8 antibody.

In another embodiment, a bispecific molecule of the present invention comprises a binding site derived from the C5E10 antibody (or a binding site which binds to the same tumor associated antigen as the C5E10 antibody). As set forth in co-pending application Ser. No. 09/104,717, C5E10 is an antibody that recognizes a glycoprotein determinant of approximately 115 kDa that appears to be specific to prostate tumor cell lines (e.g. DU145, PC3, or ND1). Thus, in conjunction with the present invention, bispecific polypeptides (e.g. CH2 domain-deleted antibodies) that specifically bind to the same tumor associated antigen recognized by C5E10 antibodies could be produced and used in a conjugated or unconjugated form for the treatment of neoplastic disorders. In particularly preferred embodiments, the binding molecule will be derived or comprise all or part of the antigen binding region of the C5E10 antibody as secreted from the hybridoma cell line having ATCC accession No. PTA-865. The resulting binding molecule could then be conjugated to a radionuclide as described below and administered to a patient suffering from prostate cancer in accordance with the methods herein.

In another embodiment, a ligand may be included in a binding molecule of the invention, e.g., to impart binding to a particular receptor or a receptor may be incorporated into a binding molecule, e.g., to remove ligands from the circulation. Exemplary ligands and their receptors that may be included in the subject bispecific binding molecules include:

a. Cytokines or Cytokine Receptors

Cytokines have pleiotropic effects on the proliferation, differentiation, and functional activation of lymphocytes. Various cytokines, or receptor binding portions thereof, can be utilized in the fusion proteins of the invention. Exemplary cytokines include the interleukins (e.g. IL-1, IL-2, IL-3, IL-4, IL-5, IL-6, IL-7, IL-8, IL-10, IL-11, IL-12, IL-13, and IL-18), the colony stimulating factors (CSFs) (e.g. granulocyte CSF (G-CSF), granulocyte-macrophage CSF (GM-CSF), and monocyte macrophage CSF (M-CSF)), tumor necrosis factor (TNF) alpha and beta, and interferons such as interferon-α, β, or γ (U.S. Pat. Nos. 4,925,793 and 4,929,554).

Cytokine receptors typically consist of a ligand-specific alpha chain and a common beta chain. Exemplary cytokine receptors include those for GM-CSF, IL-3 (U.S. Pat. No. 5,639,605), IL-4 (U.S. Pat. No. 5,599,905), IL-5 (U.S. Pat. No. 5,453,491), IFNγ (EP0240975), and the TNF family of receptors (e.g., TNFα (e.g. TNFR-1 (EP 417, 563), TNFR-2 (EP 417,014) lymphotoxin beta receptor).

b. Adhesion Proteins or their Receptors

Adhesion molecules are membrane-bound proteins that allow cells to interact with one another. Various adhesion proteins, including leukocyte homing receptors and cellular adhesion molecules, of receptor binding portions thereof, can be incorporated in a binding molecule of the invention. Leucocyte homing receptors are expressed on leucocyte cell surfaces during inflammation and include the β-1 integrins (e.g. VLA-1, 2, 3, 4, 5, and 6) which mediate binding to extracellular matrix components, and the β2-integrins (e.g. LFA-1, LPAM-1, CR3, and CR4) which bind cellular adhesion molecules (CAMs) on vascular endothelium. Exemplary CAMs include ICAM-1, ICAM-2, VCAM-1, and MAdCAM-1. Other CAMs include those of the selectin family including E-selectin, L-selectin, and P-selectin.

c. Chemokines or their Receptors

Chemokines, chemotactic proteins which stimulate the migration of leucocytes towards a site of infection, can also be incorporated into a binding molecule of the invention. Exemplary chemokines include Macrophage inflammatory proteins (MIP-1-α and MIP-1-β), neutrophil chemotactic factor, and RANTES (regulated on activation normally T-cell expressed and secreted).

d. Growth Factors or Growth Factor Receptors

Growth factors or their receptors (or receptor binding or ligand binding portions thereof) or molecules which bind to them may be incorporated in the binding molecule of the invention. Exemplary growth factors include angiopoietin, Vascular Endothelial Growth Factor (VEGF) and its isoforms (U.S. Pat. No. 5,194,596); Epidermal Growth Factors (EGFs); Fibroblastic Growth Factors (FGF), including aFGF and bFGF; atrial natriuretic factor (ANF); hepatic growth factors (HGFs; U.S. Pat. Nos. 5,227,158 and 6,099,841), neurotrophic factors such as bone-derived neurotrophic factor (BDNF), neurotrophin-3, -4, -5, or -6 (NT-3, NT-4, NT-5, or NT-6), or a nerve growth factor such as NGF-βplatelet-derived growth factor (PDGF) (U.S. Pat. Nos. 4,889,919, 4,845,075, 5,910,574, and 5,877,016); transforming growth factors (TGF) such as TGF-alpha and TGF-beta (WO 90/14359), osteoinductive factors including bone morphogenetic protein (BMP); insulin-like growth factors-I and -II (IGF-I and IGF-II; U.S. Pat. Nos. 6,403,764 and 6,506,874); Erythropoietin (EPO); stem-cell factor (SCF), thrombopoietin (c-Mpl ligand), and the Wnt polypeptides (U.S. Pat. No. 6,159,462).

Exemplary growth factor receptors which may be used include EGF receptors (EGFRs); VEGF receptors (e.g. Flt1 or Flk1/KDR), PDGF receptors (WO 90/14425); HGF receptors (U.S. Pat. Nos. 5,648,273, and 5,686,292); IGF receptors (e.g. IGFR1 and IGFR2) and neurotrophic receptors including the low affinity receptor (LNGFR), also termed as p75^{NTR} or p75, which binds NGF, BDNF, and NT-3, and high affinity receptors that are members of the trk family of the receptor tyrosine kinases (e.g. trkA, trkB (EP 455,460), trkC (EP 522,530)). In another embodiment, both IGFR1 and VEGF are targeted. In yet another embodiment, VLA4 and VEGF are targeted.

Other cell surface receptors and/or their ligands can also be targeted (e.g., the TNF family receptors or their ligands (as described in more detail herein).

e. Hormones

Exemplary growth hormones or molecules which bind to them for use as targeting agents in the binding molecule of the invention include renin, human growth hormone (HGH; U.S. Pat. No. 5,834,598), N-methionyl human growth hormone; bovine growth hormone; growth hormone releasing factor; parathyroid hormone (PTH); thyroid stimulating hormone (TSH); thyroxine; proinsulin and insulin (U.S. Pat. Nos. 5,157,021 and 6,576,608); follicle stimulating hormone (FSH), calcitonin, luteinizing hormone (LH), leptin, glucagons; bombesin; somatropin; mullerian-inhibiting substance; relaxin and prorelaxin; gonadotropin-associated peptide; prolactin; placental lactogen; OB protein; or mullerian-inhibiting substance.

f. Clotting Factors

Exemplary blood coagulation factors for use as targeting agents in the binding molecules of the invention include the clotting factors (e.g., factors V, VII, VIII, X, IX, XI, XII and XIII, von Willebrand factor); tissue factor (U.S. Pat. Nos. 5,346,991, 5,349,991, 5,726,147, and 6,596,84); thrombin and prothrombin; fibrin and fibrinogen; plasmin and plasminogen; plasminogen activators, such as urokinase or human urine or tissue-type plasminogen activator (t-PA).

C. Fusion Proteins

The invention also pertains to binding molecules which comprise one or more immunoglobulin domains. In one embodiment, the fusion proteins of the invention comprise a binding domain (which comprises at least one binding site) and a dimerization domain (which comprises at least one heavy chain portion). For example, in one embodiment, a binding molecule of the invention may comprise at least one humanized B3F6 binding site and a dimerization domain. In one embodiment, the subject fusion proteins are bispecific (with one binding site for a first target and a second binding site for a second target). In one embodiment, the subject fusion proteins are multivalent (with two binding sites for the same target).

In one embodiment a fusion protein comprises a B3F6 binding site, at least one heavy chain domain and a synthetic connecting peptide.

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

In one embodiment, when preparing a fusion proteins of the present invention, nucleic acid encoding a binding domain (e.g., a humanized B3F6 binding domain) will be fused C-terminally to nucleic acid encoding the N-terminus of an immunoglobulin constant domain sequence. N-terminal fusions are also possible. In one embodiment, a fusion protein includes a CH2 and a CH3 domain. Fusions may also be made to the C-terminus of the Fc portion of a constant domain, or immediately N-terminal to the CH1 of the heavy chain or the corresponding region of the light chain.

In one embodiment, the sequence of the ligand or receptor domain is fused to the N-terminus of the Fc domain of an immunoglobulin molecule. It is also possible to fuse the entire heavy chain constant region to the sequence of the ligand or receptor domain. In one embodiment, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. The precise site at which the fusion is made is not critical; particular sites are well known and may be selected in order to optimize the biological activity, secretion, or binding characteristics of the molecule. Methods for making fusion proteins are known in the art.

For bispecific fusion proteins, the fusion proteins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Fusion proteins are taught, e.g., in WO0069913A1 and WO0040615A2. Fusion proteins can be prepared using methods that are well known in the art (see for example U.S. Pat. Nos. 5,116,964 and 5,225,538). Ordinarily, the ligand or receptor domain is fused C-terminally to the N-terminus of the constant region of the heavy chain (or heavy chain portion) and in place of the variable region. Any transmembrane regions or lipid or phospholipids anchor recognition sequences of ligand binding receptor are preferably inactivated or deleted prior to fusion. DNA encoding the ligand or receptor domain is cleaved by a restriction enzyme at or proximal to the 5′ and 3′ ends of the DNA encoding the desired ORF segment. The resultant DNA fragment is then readily inserted into DNA encoding a heavy chain constant region. The precise site at which the fusion is made may be selected empirically to optimize the secretion or binding characteristics of the soluble fusion protein. DNA encoding the fusion protein is then transfected into a host cell for expression.

### III. Synthetic Connecting Peptides

In one embodiment, at least one polypeptide chain of a dimer of the invention comprises a synthetic connecting peptide. In one embodiment, at least two chains of a dimer of the invention comprise a connecting peptide. In a preferred embodiment, two chains of a dimer of the invention comprise a connecting peptide.

In one embodiment, connecting peptides can be used to join two heavy chain portions in frame in a single polypeptide chain. For example, in one embodiment, a connecting peptide of the invention can be used to fuse a CH3 domain (or synthetic CH3 domain) to a hinge region (or synthetic hinge region). In another embodiment, a connecting peptide of the invention can be used to fuse a CH3 domain (or synthetic CH3 domain) to a CH1 domain (or synthetic CH1 domain). In still another embodiment, a connecting peptide can act as a peptide spacer between the hinge region (or synthetic hinge region) and a CH2 domain (or a synthetic CH2 domain).

In another embodiment, a CH3 domain can be fused to an extracellular protein domain (e.g., a VL domain (or synthetic domain), a VH domain (or synthetic domain), a CH1 domain (or synthetic domain), a hinge domain (or synthetic hinge), or to the ligand binding portion of a receptor or the receptor binding portion of a ligand). For example, in one embodiment, a VH or VL domain is fused to a CH3 domain via a connecting peptide (the C-terminus of the connecting peptide is attached to the N-terminus of the CH3 domain and the N-terminus of the connecting peptide is attached to the C-terminus of the VH or VL domain). In another embodiment, a CH1 domain is fused to a CH3 domain via a connecting peptide (the C-terminus of the connecting peptide is attached to the N-terminus of the CH3 domain and the N-terminus of the connecting peptide is attached to the C-terminus of the CH1 domain). In another embodiment, a connecting peptide of the invention can be used to fuse a CH3 domain (or synthetic CH3 domain) to a hinge region (or synthetic hinge region) or portion thereof. In still another embodiment, a connecting peptide can act as a peptide spacer between the hinge region (or synthetic hinge region) and a CH2 domain (or a synthetic CH2 domain).

In one embodiment, a connecting peptide can comprise or consist of a gly/ser spacer. For example, a domain deleted construct having a short amino acid spacer GGSSGGGGSG (SEQ ID No. 8) substituted for the CH2 domain and the lower hinge region (CH2 [gly/ser]) can be used. In another embodiment, a connecting peptide comprises the amino acid sequence IGKTISKKAK (SEQ ID NO:15).

In another embodiment, connecting peptide can comprise at least a portion of an immunoglobulin hinge region. Hinge domains can be subdivided into three distinct regions: upper, middle, and lower hinge regions (Roux et al. J. Immunol. 1998 161:4083). Polypeptide sequences encompassing these regions for IgG1 and IgG3 hinges are shown in Table 3. For example, chimeric hinge domains can be constructed which combine hinge elements derived from different antibody isotypes. In one embodiment, a connecting peptide comprises at least a portion of an IgG1 hinge region. In another embodiment, a connecting peptide can comprise at least a portion of an IgG3 hinge region. In another embodiment, a connecting peptide can comprise at least a portion of an IgG1 hinge region and at least a portion of an IgG3 hinge region. In one embodiment, a connecting peptide can comprise an IgG1 upper and middle hinge and a single IgG3 middle hinge repeat motif.

TABLE 3
IgG1, IgG3 and IgG4 Hinge Regions

| | | | Lower |
|---|---|---|---|
| IgG | Upper Hinge | Middle Hinge | Hinge |
| IgG1 | EPKSCDKTHT | CPPCP | APELLGGP |
| | (SEQ ID NO: | (SEQ ID NO: 18) | (SEQ ID |
| | 17) | | NO: 19) |
| IgG3 | ELKTPLGDTTHT | CPRCP(EPKSCDTPPPCPRCP)₃ | APELLGGP |
| | (SEQ ID NO: | (SEQ ID NO: 21) | (SEQ ID |
| | 20) | | NO: 19) |
| IgG4 | ESKYGPP | CPSCP | APEFLGGP |
| | (SEQ ID NO: | (SEQ ID NO: 23) | (SEQ ID |
| | 22) | | NO: 24) |

Exemplary connecting peptides are taught, for example, in WO 06/74397.

In one embodiment, a connecting peptide of the invention comprises a non-naturally occurring immunoglobulin hinge region domain, e.g., a hinge region domain that is not naturally found in the polypeptide comprising the hinge region domain and/or a hinge region domain that has been altered so that it differs in amino acid sequence from a naturally occurring immunoglobulin hinge region domain. In one embodiment, mutations can be made to hinge region domains to make a connecting peptide of the invention. In one embodiment, a connecting peptide of the invention comprises a hinge domain which does not comprise a naturally occurring number of cysteines, i.e., the connecting peptide comprises either fewer cysteines or a greater number of cysteines than a naturally occurring hinge molecule. In a preferred embodiment, incorporation of the connecting peptide into a polypeptide results in a composition in which greater than 50%, 60%, 70%, 80% or 90% of the dimeric molecules present in a form in which the two heavy chain portions are linked via at least one interchain disulfide linkage.

In one embodiment of the invention, a connecting peptide comprises hinge region domain comprising a proline residue at an amino acid position corresponding to amino acid position 243 in the Kabat numbering system (position 230, EU numbering system). In one embodiment, a connecting peptide comprises an alanine residue at an amino acid position corresponding to position 244, Kabat numbering system (position 246, EU numbering system). In another embodiment, a connecting peptide of the invention comprises a proline residue at an amino acid position corresponding to position 245 (Kabat numbering system; position 247, EU numbering system)). In one embodiment, a connecting peptide comprises a cysteine residue at an amino acid position corresponding to position 239, Kabat numbering system (position 226, EU numbering system). In one embodiment, a connecting peptide comprises a serine residue at an amino acid position corresponding to position 239, Kabat numbering system (position 226, EU numbering system). In one embodiment, a connecting peptide comprises a cysteine residue at an amino acid position corresponding to position 242, Kabat numbering system (position 229, EU numbering system). In one embodiment, a connecting peptide comprises a serine residue at an amino acid position corresponding to position 242, Kabat numbering system (position 229, EU numbering system).

In one embodiment, the connecting peptide can be chosen to result in the preferential synthesis of a particular isoform of polypeptide, e.g., in which the two heavy chain portions are linked via disulfide bonds or are not linked via disulfide bonds. For example, as described in the examples of WO 2006 074397, the G1/G3/Pro243+[gly/ser] linker (SEQ ID NO: 26), G1/G3/Pro243Ala244Pro245+[gly/ser] linker (SEQ ID NO: 5), Pro243+[gly/ser] linker (SEQ ID NO:33), and Pro243Ala244Pro245+[gly/ser] linker (SEQ ID NO: 32), connecting peptides resulted in the production of only Form A CH2 domain-deleted antibody with no detectable Form B. In contrast, CH2 domain-deleted Cys242Ser:Pro243 (SEQ ID NO: 31), and CH2 domain-deleted Cys242Ser:Pro243Ala244Pro245 (SEQ ID NO: 32), both resulted in a preference for the Form B isoform. These synthetic hinge region connecting peptides would thus be useful for favoring synthesis of Form A or B isoform. This is true for any isotype of antibody, (e.g., IgG1, IgG2, IgG3, or IgG4) based on the high degree of homology among the CH3 domains for all four human isotypes. (Including identical and conserved amino acid residues, IgG1 CH3 domain is 98.13% homologous to IgG2 CH3, 97.20% homologous to IgG3 CH3, and 96.26% homologous to IgG4 CH3). The parentheticals referring to connecting peptides and various binding molecules of the invention represent equivalent terminology unless otherwise indicated.

In one embodiment, a connecting peptide of the invention comprises a hinge region domain followed by a flexible gly/ser linker. Exemplary connecting peptides are shown in Table 2 and in SEQ ID NOs: 5, 25-34. It will be understood that variant forms of these exemplary connecting peptides can be created by introducing one or more nucleotide substitutions, additions or deletions into the nucleotide sequence encoding a connecting peptide such that one or more amino acid substitutions, additions or deletions are introduced into the connecting peptide. For example, mutations may be introduced by standard techniques, such as site-directed mutagenesis and PCR-mediated mutagenesis. Preferably, conservative amino acid substitutions are made at one or more non-essential amino acid residues such that the ability of the connecting peptide to preferentially enhance synthesis of Form A or Form B is not altered. Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

TABLE 2
Hinge Region Connecting Peptide Sequences

| Kabat | | | | | | | | | | | | | | | | | | | |
|---|---|---|---|---|---|---|---|---|---|---|---|---|---|---|---|---|---|---|---|
| hinge position: | 226 | 227 | 228 | 229 | 230 | 232 | 235 | 236 | 237 | 238 | 239 | 240 | 241 | 241EE | 241FF | 241GG | 241HH | 241II | 241JJ |
| IgG1 hinge sequence | E | P | K | S | C | D | K | T | H | T | C | P | P | | | | | | |
| (SEQ ID NO: 36) | | | | | | | | | | | | | | | | | | | |
| IgG4 hinge sequence | E | S | K | Y | G | | | | P | P | C | P | S | | | | | | |
| (SEQ ID NOs: 37 and | | | | | | | | | | | | | | | | | | | |
| 38) | | | | | | | | | | | | | | | | | | | |
| IgG3 middle hinge | | | | | | | | | | | | | | C | P | E | P | K | S |
| sequence (SEQ ID | | | | | | | | | | | | | | | | | | | |
| NO: 35) | | | | | | | | | | | | | | | | | | | |
| Connecting peptide: | Connecting peptide sequences | | | | | | | | | | | | | | | | | | |
| G1 | E | P | K | S | C | D | K | T | H | T | C | P | P | | | | | | |
| | (Seq. ID NO: 25) | | | | | | | | | | | | | | | | | | |
| G1/G3/Pro243 | E | P | K | S | C | D | K | T | H | T | C | P | P | C | P | E | P | K | S |
| | (Seq. ID NO: 26) | | | | | | | | | | | | | | | | | | |
| G1/G3/ | E | P | K | S | C | D | K | T | H | T | C | P | P | C | P | E | P | K | S |
| Pro243Ala244Pro245 | (Seq. ID NO: 27) | | | | | | | | | | | | | | | | | | |
| G1/Cys239Ser:Pro243 | E | P | K | S | C | D | K | T | H | T | S | P | P | | | | | | |
| | (Seq. ID NO: 28) | | | | | | | | | | | | | | | | | | |
| G1/Cys239Ser:Pro243 | E | P | K | S | C | D | K | T | H | T | S | P | P | | | | | | |
| Ala244Pro245 | (Seq. ID NO: 29) | | | | | | | | | | | | | | | | | | |
| G1/Cys242Ser:Pro243 | E | P | K | S | C | D | K | T | H | T | C | P | P | | | | | | |
| | (Seq. ID NO: 30) | | | | | | | | | | | | | | | | | | |
| G1/Cys242Ser:Pro243 | E | P | K | S | C | D | K | T | H | T | C | P | P | | | | | | |
| Ala244Pro245 | (Seq. ID NO: 31) | | | | | | | | | | | | | | | | | | |
| G1/ | E | P | K | S | C | D | K | T | H | T | C | P | P | | | | | | |
| Pro243Ala244Pro245 | (Seq. ID NO: 32) | | | | | | | | | | | | | | | | | | |
| G1/Pro243 | E | P | K | S | C | D | K | T | H | T | C | P | P | | | | | | |
| | (Seq. ID NO: 33) | | | | | | | | | | | | | | | | | | |
| G4/G3/ | E | S | K | Y | G | | | | P | P | C | P | S | C | P | E | P | K | S |

| Pro243Ala244Pro245 | (Seq. ID NO: 34) | | | | | | | | | | | | | |
|---|---|---|---|---|---|---|---|---|---|---|---|---|---|---|
| Kabat | | | | | | | | | | | | | | |
| hinge position: | 241KK | 241LL | 241MM | 241NN | 241OO | 241PP | 241OO | 241RR | 241SS | 242 | 243 | 244 | 245 | |
| IgG1 hinge sequence | | | | | | | | | | C | P | A | P | |
| (SEQ ID NO: 36) | | | | | | | | | | | | | | |
| IgG4 hinge sequence | | | | | | | | | | C | P | A | P | |
| (SEQ ID NOs: 37 and | | | | | | | | | | | | | | |
| 38) | | | | | | | | | | | | | | |
| IgG3 middle hinge | C | D | T | P | P | P | C | P | R | | | | | |
| sequence (SEQ ID | | | | | | | | | | | | | | |
| NO: 35) | | | | | | | | | | | | | | |
| Connecting peptide: | Connecting peptide sequences | | | | | | | | | | | | | |
| G1 | | | | | | | | | | C | | | | GGGSSGGGSG |
| G1/G3/Pro243 | C | D | T | P | P | P | C | P | R | C | P | | | GGGSSGGGSG |
| G1/G3/ | C | D | T | P | P | P | C | P | R | C | P | A | P | GGGSSGGGSG |
| Pro243Ala244Pro245 | | | | | | | | | | | | | | |
| G1/Cys239Ser:Pro243 | | | | | | | | | | C | P | | | GGGSSGGGSG |
| G1/Cys239Ser:Pro243 | | | | | | | | | | C | P | A | P | GGGSSGGGSG |
| Ala244Pro245 | | | | | | | | | | | | | | |
| G1/Cys242Ser:Pro243 | | | | | | | | | | S | P | | | GGGSSGGGSG |
| G1/Cys242Ser:Pro243 | | | | | | | | | | S | P | A | P | GGGSSGGGSG |
| Ala244Pro245 | | | | | | | | | | | | | | |
| G1/ | | | | | | | | | | C | P | A | P | GGGSSGGGSG |
| Pro243Ala244Pro245 | | | | | | | | | | | | | | |
| G1/Pro243 | | | | | | | | | | C | P | | | GGGSSGGGSG |
| G4/G3/ | C | D | T | P | P | P | C | P | R | C | P | A | P | |
| Pro243Ala244Pro245 | | | | | | | | | | | | | | |

Connecting peptides of the invention can be of varying lengths. In one embodiment, a connecting peptide of the invention is from about 15 to about 50 amino acids in length. In another embodiment, a connecting peptide of the invention is from about 20 to about 45 amino acids in length. In another embodiment, a connecting peptide of the invention is from about 25 to about 40 amino acids in length. In another embodiment, a connecting peptide of the invention is from about 30 to about 35 amino acids in length. In another embodiment, a connecting peptide of the invention is from about 24 to about 27 amino acids in length. In another embodiment, a connecting peptide of the invention is from about 40 to about 42 amino acids in length.

Connecting peptides can be introduced into polypeptide sequences using techniques known in the art. For example, in one embodiment, the Splicing by Overlap Extension (SOE) method (Horton, R. M. 1993 Methods in Molecular Biology, Vol 15:PCR Protocols: Current Methods and applications. Ed. B. A. White) can be used. Modifications can be confirmed by DNA sequence analysis. Plasmid DNA can be used to transform host cells for stable production of the polypeptides produced.

In one embodiment, incorporation of one of the subject connecting peptides into a polypeptide yields a composition comprising binding molecules having at least two binding sites and at least two polypeptide chains, wherein at least two of the polypeptide chains comprise a synthetic connecting peptide and wherein greater than 50% of the molecules are present in a form in which the two heavy chain portions are linked via at least one interchain disulfide linkage. In another embodiment, greater than 60% of the molecules are present in a form in which the two heavy chain portions are linked via at least one interchain disulfide linkage. In another embodiment, greater than 70% of the molecules are present in a form in which the two heavy chain portions are linked via at least one interchain disulfide linkage. In another embodiment, greater than 80% of the molecules are present in a form in which the two heavy chain portions are linked via at least one interchain disulfide linkage. In another embodiment, greater than 90% of the molecules are present in a form in which the two heavy chain portions are linked via at least one interchain disulfide linkage.

### IV. Expression of Binding Molecules

Following manipulation of the isolated genetic material to provide polypeptides of the invention as set forth above, the genes are typically inserted in an expression vector for introduction into host cells that may be used to produce the desired quantity of polypeptide that, in turn, provides the claimed binding molecules.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals. In particularly preferred embodiments the cloned variable region genes are inserted into an expression vector along with the heavy and light chain constant region genes (preferably human) synthetic as discussed above. Preferably, this is effected using a proprietary expression vector of IDEC, Inc., referred to as NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. As seen in the examples of WO 2006 074397, this vector has been found to result in very high level expression of antibodies upon incorporation of variable and constant region genes, transfection in CHO cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the polypeptides of the invention may be expressed using polycistronic constructs such as those disclosed in copending U.S. provisional application No. 60/331,481 filed Nov. 16, 2001 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of polypeptides of the invention in eukaryotic host cells. Compatible IRES sequences are disclosed in U.S. Pat. No. 6,193,980 which is also incorporated herein. Those skilled in the art will appreciate that such expression systems may be used to effectively produce the full range of polypeptides disclosed in the instant application.

More generally, once the vector or DNA sequence encoding a monomeric subunit of the polypeptide (e.g. a modified antibody) has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding the polypeptide of the invention can also be expressed non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules. Where tetravalent forms of antibodies are desired, the subunits will then self-assemble into tetravalent antibodies (WO02/096948A2).

In addition to prokaryates, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### V. Labeling or Conjugation of Binding Molecules

The binding molecules of the present invention may be used in non-conjugated form or may be conjugated to at least one of a variety of effector, i.e., functional, moieties, e.g., to facilitate target detection or for imaging or therapy of the patient. The polypeptides of the invention can be labeled or conjugated either before or after purification, when purification is performed. In particular, the polypeptides of the present invention may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), PEG, or detectable moieties useful in imaging. In another embodiment, a polypeptide of the invention can be conjugated to a molecule that decreases vascularization of tumors. In other embodiments, the disclosed compositions may comprise polypeptides of the invention coupled to drugs or prodrugs. Still other embodiments of the present invention comprise the use of polypeptides of the invention conjugated to specific biotoxins or their cytotoxic fragments such as ricin, gelonin, pseudomonas exotoxin or diphtheria toxin. The selection of which conjugated or unconjugated polypeptide to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukemias in animal models, and in some cases in humans. Exemplary radioisotopes include: ⁹⁰Y, ¹²⁵I, ¹³¹I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, polypeptides of the invention may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99m labeled polypeptides may be prepared by ligand exchange processes, by reducing pertechnate (TcO₄⁻) with stannous ion solution, chelating the reduced technetium onto a Sephadex column and applying the polypeptides to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I covalently attached via tyrosine residues. Polypeptides according to the invention may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose. However, for the purposes of the present invention, the indirect labeling approach is particularly preferred. Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein in their entirety. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation in co-pending application Ser. Nos. 08/475,813, 08/475,815 and 08/478,967, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy. It will also be appreciated that, in accordance with the teachings herein, polypeptides may be conjugated to different radiolabels for diagnostic and therapeutic purposes. To this end the aforementioned co-pending applications, herein incorporated by reference in their entirety, disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunction al chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the art can readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target molecule.

Those skilled in the art will appreciate that these non-radioactive conjugates may also be assembled using a variety of techniques depending on the selected agent to be conjugated. For example, conjugates with biotin are prepared e.g. by reacting the polypeptides with an activated ester of biotin such as the biotin N-hydroxysuccinimide ester. Similarly, conjugates with a fluorescent marker may be prepared in the presence of a coupling agent, e.g. those listed above, or by reaction with an isothiocyanate, preferably fluorescein-isothiocyanate. Conjugates of the polypeptides of the invention with cytostatic/cytotoxic substances and metal chelates are prepared in an analogous manner.

Many effector moieties lack suitable functional groups to which antibodies can be linked. In one embodiment, an effector moiety, e.g., a drug or prodrug is attached to the antibody through a linking moiety. In one embodiment, the linking moiety contains a chemical bond that allows for the activation of cytotoxicity at a particular site. Suitable chemical bonds are well known in the art and include disulfide bonds, acid labile bonds, photolabile bonds, peptidase labile bonds, thioether bonds formed between sulfhydryl and maleimide groups, and esterase labile bonds. Most preferably, the linking moiety comprises a disulfide bond or a thioether bond. In accordance with the invention, the linking moiety preferably comprises a reactive chemical group. Particularly preferred reactive chemical groups are N-succinimidyl esters and N-sulfosuccinimidyl esters. In a preferred embodiment, the reactive chemical group can be covalently bound to the effector via disulfide bonding between thiol groups. In one embodiment an effector molecule is modified to comprise a thiol group. One of ordinary skill in the art will appreciate that a thiol group contains a sulfur atom bonded to a hydrogen atom and is typically also referred to in the art as a sulfhydryl group, which can be denoted as “—SH” or “RSH.”

In one embodiment, a linking moiety may be used to join the effector moiety with the binding molecule. The linking moiety of the invention may be cleavable or non-cleavable. In one embodiment, the cleavable linking moiety is a redox-cleavable linking moiety, such that the linking moiety is cleavable in environments with a lower redox potential, such as the cytoplasm and other regions with higher concentrations of molecules with free sulfhydryl groups. Examples of linking moieties that may be cleaved due to a change in redox potential include those containing disulfides. The cleaving stimulus can be provided upon intracellular uptake of the binding protein of the invention where the lower redox potential of the cytoplasm facilitates cleavage of the linking moiety. In another embodiment, a decrease in pH triggers the release of the maytansinoid cargo into the target cell. The decrease in pH is implicated in many physiological and pathological processes, such as endosome trafficking, tumor growth, inflammation, and myocardial ischemia. The pH drops from a physiological 7.4 to 5-6 in endosomes or 4-5 in lysosomes. Examples of acid sensitive linking moieties which may be used to target lysosomes or endosomes of cancer cells, include those with acid-cleavable bonds such as those found in acetals, ketals, orthoesters, hydrazones, trityls, cis-aconityls, or thiocarbamoyls (see for example, Willner et al., (1993), Bioconj. Chem., 4: 521-7; U.S. Pat. Nos. 4,569,789, 4,631,190, 5,306,809, and 5,665,358). Other exemplary acid-sensitive linking moieties comprise dipeptide sequences Phe-Lys and Val-Lys (King et al., (2002), J. Med. Chem., 45: 4336-43). The cleaving stimulus can be provided upon intracellular uptake trafficking to low pH endosomal compartments (e.g. lysosomes). Other exemplary acid-cleavable linking moieties are the moieties that contain two or more acid cleavable bonds for attachment of two or more maytansinoids (King et al., (1999), Bioconj. Chem., 10: 279-88; WO 98/19705).

Cleavable linking moieties may be sensitive to biologically supplied cleaving agents that are associated with a particular target cell, for example, lysosomal or tumor-associated enzymes. Examples of linking moieties that can be cleaved enzymatically include, but are not limited to, peptides and esters. Exemplary enzyme cleavable linking moieties include those that are sensitive to tumor-associated proteases such as Cathepsin B or plasmin (Dubowchik et al., (1999), Pharm. Ther., 83: 67-123; Dubowchik et al., (1998), Bioorg. Med. Chem. Lett., 8: 3341-52; de Groot et al., (2000), J. Med. Chem., 43: 3093-102; de Groot et al., (1999)m 42: 5277-83). Cathepsin B-cleavable sites include the dipeptide sequences valine-citrulline and phenylalanine-lysine (Doronina et al., (2003), Nat. Biotech., 21(7): 778-84); Dubowchik et al., (2002), Bioconjug. Chem., 13: 855-69). Other exemplary enzyme-cleavable sites include those formed by oligopeptide sequences of 4 to 16 amino acids (e.g., Suc-β-Ala-Leu-Ala-Leu) which recognized by trouse proteases such as Thimet Oliogopeptidase (TOP), an enzyme that is preferentially released by neutrophils, macrophages, and other granulocytes.

In a further embodiment, the linking moiety is formed by reacting a binding molecule of the invention with a linking molecule of the formula:

X-Y-Z

wherein:
X is an attachment moiety;
Y is a spacer moiety; and
Z is a effector attachment moeity.

The term “binding molecule attachment moiety” includes moieties which allow for the covalent attachment of the linker to a binding molecule of the invention.

The attachment moiety may comprise, for example, a covalent chain of 1-60 carbon, oxygen, nitrogen, sulfur atoms, optionally substituted with hydrogen atoms and other substituents which allow the binding molecule to perform its intended function. The attachment moiety may comprise peptide, ester, alkyl, alkenyl, alkynyl, aryl, ether, thioether, etc. functional groups. Preferably, the attachment moiety is selected such that it is capable of reacting with a reactive functional group on a polypeptide comprising at least one antigen binding site, to form a binding molecule of the invention. Examples of attachment moieties include, for example, amino, carboxylate, and thiol attachment moieties.

Amino attachment moieties include moieties which react with amino groups on a polypeptide, such that a binding molecule of the invention is formed. Amino attachment moieties are known in the art. Examples of amino attachment moieties include, activated carbamides (e.g., which may react with an amino group on a binding molecule to form a linking moiety which comprises urea group), aldehydes (e.g., which may react with amino groups on a binding molecule), and activated isocyanates (which may react with an amino group on a binding molecule to from a linking moiety which comprises a urea group). Examples of amino attachment moieties include, but are not limited to, N-succinimidyl, N-sulfosuccinimidyl, N-phthalimidyl, N-sulfophthalimidyl, 2-nitrophenyl, 4-nitrophenyl, 2,4-dinitrophenyl, 3-sulfonyl-4-nitrophenyl, or 3-carboxy-4-nitrophenyl moiety.

Carboxylate attachment moieties include moieties which react with carboxylate groups on a polypeptide, such that a binding molecule of the invention is formed. Carboxylate attachment moieties are known in the art. Examples of carboxylate attachment moieties include, but are not limited to activated ester intermediates and activated carbonyl intermediates, which may react with a COOH group on a binding molecule to form a linking moiety which comprises a ester, thioester, or amide group.

Thiol attachment moieties include moieties which react with thiol groups present on a polypeptide, such that a binding molecule of the invention is formed. Thiol attachment moieties are known in the art. Examples of thiol attachment moieties include activated acyl groups (which may react with a sulfhydryl on a binding molecule to form a linking moiety which comprises a thioester), activated alkyl groups (which may react with a sulfhydryl on a binding molecule to form a linking moiety which comprises a thioester moiety), Michael acceptors such as maleimide or acrylic groups (which may react with a sulfhydryl on a binding molecule to form a Michael-type addition product), groups which react with sulfhydryl groups via redox reactions, activated di-sulfide groups (which may react with a sulfhydryl group on a binding molecule to form, for example, a linking moiety which comprises a disulfide moiety). Other thiol attachment moieties include acrylamides, alpha-iodoacetamides, and cyclopropan-1,1-dicarbonyl compounds. In addition, the thiol attachment moiety may comprise a moiety which modifies a thiol on the binding molecule to form another reactive species to which the linking molecule can be attached to form a binding molecule of the invention.

The spacer moiety, Y, is a covalent bond or a covalent chain of atoms which may contain one or more amino acid residues. It may also comprise 0-60 carbon, oxygen, sulfur or nitrogen atoms optionally substituted with hydrogen or other substituents which allow the resulting binding molecule to perform its intended function. In one embodiment, Y comprises an alkyl, alkenyl, alkynyl, ester, ether, carbonyl, or amide moiety.

In another embodiment, a thiol group on the binding molecule is converted into a reactive group, such as a reactive carbonyl group, such as a ketone or aldehyde. The attachment moiety is then reacted with the ketone or aldehyde to form the desired compound of the invention. Examples of carbonyl reactive attachment moieties include, but are not limited to, hydrazines, hydrazides, O-substituted hydroxylamines, alpha-beta-unsaturated ketones, and H₂C═CH—CO—NH—NH₂. Other examples of attachment moieties and methods for modifying thiol moieties which can be used to form binding molecules of the invention are described Pratt, M. L. et al. J Am Chem Soc. 2003 May 21; 125(20):6149-59; and Saxon, E. Science. 2000 Mar. 17; 287(5460):2007-10.

The linking molecule may be a molecule which is capable of reacting with an effector moiety or a derivative thereof to form a binding molecule of the invention. For example, the effector moiety may be linked to the remaining portions of the molecule through a disulfide bond. In such cases, the linking moiety is selected such that it is capable of reacting with an appropriate effector moeity derivative such that the effector moiety is attached to the binding molecule of the invention. As described above, the linking moiety and/or the linker as a whole may be selected that the linker is cleaved in an appropriate environment.

Particularly preferred linker molecules include, for example, N-succinimidyl 3-(2-pyridyldithio)propionate (SPDP) (see, e.g., Carlsson et al., Biochem. J., 173, 723-737 (1978)), N-succinimidyl 4-(2-pyridyldithio)butanoate (SPDB) (see, e.g., U.S. Pat. No. 4,563,304), N-succinimidyl 4-(2-pyridyldithio)pentanoate (SPP) (see, e.g., CAS Registry number 341498-08-6), N-succinimidyl 4-(N-maleimidomethyl)cyclohexane-1-carboxylate (SMCC) (see, e.g., Yoshitake et al., Eur. J. Biochem., 101, 395-399 (1979)), and N-succinimidyl 4-methyl-4-[2-(5-nitro-pyridyl)-dithio]pentanoate (SMNP) (see, e.g., U.S. Pat. No. 4,563,304) The most preferred linker molecules for use in the inventive composition are SPP, SMCC, and SPDB. In a preferred embodiment, SPDB is used to link an effector moiety to a binding molecule of the invention.

In one embodiment of the invention, the linker molecules SPP, SMCC or SPDB are used to link an anti-Cripto binding molecule to a maytansinoid. In one embodiment, the SPDB crosslinker is used to link DM4 to an anti-Cripto binding molecule. In another embodiment, SPDB is used to link DM1 to an anti-Cripto binding molecule. In another embodiment, SMCC is used to link DM4 to an anti-Cripto binding molecule. In another embodiment, SMCC is used to link DM1 to an anti-Cripto binding molecule. In another embodiment, SPP is used to link DM4 to an anti-Cripto binding molecule. In another embodiment, SPP is used to link DM1 to an anti-Cripto binding molecule. In preferred embodiments, the anti-Cripto binding molecule is a humanized anti-Cripto antibody.

Preferred cytotoxic effector moieties for use in the present invention are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Exemplary cytotoxins include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine.

Particularly preferred moieties for conjugation are maytansinoids. Maytansinoids were originally isolated from the east African shrub belonging to the genus Maytenus, but were subsequently also discovered to be metabolites of soil bacteria, such as Actinosynnema pretiosum (see, e.g., U.S. Pat. No. 3,896,111). Maytansinoids are known in the art to include maytansine, maytansinol, C-3 esters of maytansinol, and other maytansinol analogues and derivatives (see, e.g., U.S. Pat. Nos. 5,208,020 and 6,441,163). C-3 esters of maytansinol can be naturally occurring or synthetically derived. Moreover, both naturally occurring and synthetic C-3 maytansinol esters can be classified as a C-3 ester with simple carboxylic acids, or a C-3 ester with derivatives of N-methyl-L-alanine, the latter being more cytotoxic than the former. Synthetic maytansinoid analogues also are known in the art and described in, for example, Kupchan et al., J. Med. Chem., 21, 31-37 (1978). Methods for generating maytansinol and analogues and derivatives thereof are described in, for example, U.S. Pat. No. 4,151,042.

Suitable maytansinoids for use as antibody conjugates can be isolated from natural sources, synthetically produced, or semi-synthetically produced using methods known in the art. Moreover, the maytansinoid can be modified in any suitable manner, so long as sufficient cytotoxicity is preserved in the ultimate conjugate molecule.

Particularly preferred maytansinoids comprising a linking moiety that contains a reactive chemical group are C-3 esters of maytansinol and its analogs where the linking moiety contains a disulfide bond and the attachment moiety comprises a N-succinimidyl or N-sulfosuccinimidyl ester. Many positions on maytansinoids can serve as the position to chemically link the linking moiety, e.g., through an effector attachment moiety. For example, the C-3 position having a hydroxyl group, the C-14 position modified with hydroxymethyl, the C-15 position modified with hydroxy and the C-20 position having a hydroxy group are all useful. The linking moiety most preferably is linked to the C-3 position of maytansinol. Most preferably, the maytansinoid used in connection with the inventive compositions and methods is N.sup.2′-deacetyl-N.sup.2′-(-3-mercapto-1-oxopropyl)-maytansine (DM1) or N.sup.2′-deacetyl-N.sup.2′-(4-mercapto-4-methyl-1-oxopentyl)-maytansine (DM4). These various linking moieties are known to release the conjugated antibody with different half-lives in the human body. In particular, the SPP-DM1 linker conjugate has a half life of approximately 24-48 hours in man, the SPDB-DM4 linker conjugate has a half life of approximately 5 days in man, and the SMCC-DM1 linker conjugate has a half life of approximately 6 days in man. In particular, the SPP and SPDB linkers produce metabolites that can re-enter neighboring tumor cells, producing a so-called “bystander” effect that can contribute to tumor cell killing. In contrast, SMCC-DM 1 linker system does not produce a metabolite product that can re-enter neighboring tumor cells. Accordingly, antibody conjugates comprising the SMCC-DM1 linker system, e.g., B3F6-SMCC-DM1, are useful in the treatment of tumors that do not require the “bystander” killing activity. Antibody conjugates comprising the SPDB-DM4 linker system, e.g., B3F6-SPDB-DM4, is useful in inhibiting tumor growth in both tumors that do and do not require the “bystander” killing activity.

Linking moieties with other chemical bonds also can be used in the context of the invention, as can other maytansinoids. Specific examples of other chemical bonds which may be incorporated in the linking moieties include those described above, such as, for example acid labile bonds, thioether bonds, photolabile bonds, peptidase labile bonds and esterase labile bonds. Methods for producing maytansinoids with linking moieties and/or effector attachment moieties are described in, for example, U.S. Pat. Nos. 5,208,020, 5,416,064, and 6,333,410.

The linking moiety (and/or the effector attachment moiety) of a maytansinoid typically and preferably is part of a larger linker molecule that is used to join the antibody to the maytansinoid. Any suitable linker molecule can be used in connection with the invention, so long as the linking molecule provides for retention of the cytotoxicity and targeting characteristics of the maytansinoid and the antibody, respectively. The linking molecule joins the maytansinoid to the antibody through chemical bonds (as described above), such that the maytansinoid and the antibody are chemically coupled (e.g., covalently bonded) to each other. Desirably, the linking molecule chemically couples the maytansinoid to the antibody through disulfide bonds or thioether bonds. Most preferably, the antibody is chemically coupled to the maytansinoid via disulfide bonds.

Preferred conjugated binding molecules of the invention are anti-Cripto antibodies conjugated to a maytansinoid, e.g., DM4 or DM1. Preferred anti-Cripto antibody-maytansinoid conjugates of the invention have an average of between about 0.5 and 10 molecules of maytansinoid, e.g., DM4, attached to one molecule of antibody. Preferably, there is an average of between about 1 and 8 molecules of maytansinoid, e.g., DM4, attached to one molecule of antibody, or an average of between about 2 and 6 molecules of maytansinoid, e.g., DM4, attached to one molecule of antibody. Preferably, there is an average of between about 3 and 5 molecules of maytansinoid, e.g., DM4, and more preferably, an average of between about 3 and 4 molecules of maytansinoid, e.g., DM4, attached to one molecule of antibody. In preferred embodiments, there is an average of about 3.0, 3.1, 3.2, 3.3, 3.4, 3.5, 3.6, 3.7, 3.8, 3.9 or 4.0 molecules of maytansinoid, e.g., DM4, attached to one molecule of antibody. In a particularly preferred embodiment, anti-Cripto antibody-maytansinoid conjugates of the invention have an average of about 3.5 molecules of maytansinoid, e.g., DM4, attached to one molecule of antibody. In one embodiment, at least 50% of the anti-Cripto antibody-maytansinoid conjugates of the invention have 2, 3 or 4 molecules of maytansinoid, e.g., DM4.

Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, caminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. As noted previously, one skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

Among other cytotoxins, it will be appreciated that polypeptides can also be associated with a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the polypeptides of the invention of the present invention comprise cytokines such as lymphokines and interferons. In view of the instant disclosure it is submitted that one skilled in the art could readily form such constructs using conventional techniques.

Another class of compatible cytotoxins that may be used in conjunction with the disclosed polypeptides are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked polypeptides of the invention would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

In one embodiment, a moiety that enhances the stability or efficacy of the polypeptide can be conjugated. For example, in one embodiment, PEG can be conjugated to the polypeptides of the invention to increase their half-life in vivo. Leong, S. R., et al. 2001. Cytokine 16:106; 2002; Adv. in Drug Deliv. Rev. 54:531; or Weir et al. 2002. Biochem. Soc. Transactions 30:512.

As previously alluded to, compatible cytotoxins may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. In one embodiment, a cytotoxic agent, such as a maytansinoid, is administered as a prodrug which is released by the hydrolysis of disulfide bonds. Further examples of cytotoxic drugs that can be derivatized into a prodrug form for use in the present invention comprise those chemotherapeutic agents described above.

### VI. Administration of Binding Molecules

Methods of preparing and administering polypeptides of the invention to a subject are well known to or are readily determined by those skilled in the art. The route of administration of the polypeptide of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to autoimmune or neoplastic disorders.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

For passive immunization with an antibody, the dosage can range, e.g., from about 0.0001 to 100 mg/kg, and more usually 0.01 to 50 mg/kg, and even more usually 0.1 to 40 mg/kg (e.g., 0.25 mg/kg, 0.5 mg/kg, 0.75 mg/kg, 1 mg/kg, 2 mg/kg, 4 mg/kg, 8 mg/kg etc.), of the host body weight. For example dosages can be 1 mg/kg, 5 mg/kg, 10 mg/kg, 15 mg/kg, 20 mg/kg, 25, mg/kg, 30 mg/kg, 35 mg/kg, 40 mg/kg, 45 mg/kg or 50 mg/kg body weight or any dose within the range of 1-50 mg/kg, preferably at least 1 mg/kg. Doses intermediate in the above ranges are also intended to be within the scope of the invention.

Dosages can also range, for example, from 0.0037 to 3700 mg/m², and more usually from 0.37 to 1850 mg/m², and even more usually from 3.7 mg/m² to 1480 mg/m². Dosages can also range, for example, from 1 to 1000 mg/m², and more usually from 6 mg/m2 to 500 mg/m², more usually from 10 mg/m2 to 200 mg/m², and more usually from 20 to 80 mg/m², and even more usually from 50-75 mg/m², and most usually from 60-70 mg/m². Doses can also range from 24 to 90 mg/m². Doses intermediate in the above ranges are also intended to be within the scope of the invention.

Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks (biweekly), once per every three weeks, once per every four weeks, or once a month, or once every 3 to 6 months. In one embodiment of the invention, an exemplary treatment regime entails administration (e.g., of a humanized anti-Cripto antibody conjugated to a maytansinoid, e.g., B3F6.1-DM4) once per every three weeks. In a particularly preferred embodiment, the exemplary treatment regime of once per every three weeks (e.g., of a humanized anti-Cripto antibody conjugated to a maytansinoid, e.g., B3F6.1-DM4) is particularly useful in the treatment of colon cancer. In another embodiment, an exemplary treatment regime entails administration (e.g., of a humanized anti-Cripto antibody conjugated to a maytansinoid, e.g., B3F6.1-DM4) in a single dose. In a preferred embodiment, the exemplary treatment regime of a single dose (e.g., of a humanized anti-Cripto antibody conjugated to a maytansinoid, e.g., B3F6.1-DM4) is particularly useful in the treatment of established or advanced tumors. In a particularly preferred embodiment, the exemplary treatment regime of a single dose (e.g., of a humanized anti-Cripto antibody conjugated to a maytansinoid, e.g., B3F6.1-DM4) is useful in the treatment of established or advanced colon tumors.

Exemplary dosage schedules include a single dose administration at, e.g., 5 mg/kg, 10 mg/kg, 15 mg/kg, 20 mg/kg, 25 mg/kg, 30 mg/kg or 40 mg/kg. Exemplary dosage schedules further include a biweekly dose at, e.g., 25-40 mg/kg. Dosage schedules include a biweekly dose at, e.g., 5 mg/kg, 10 mg/kg, 15 mg/kg, 20 mg/kg, 25 mg/kg, 30 mg/kg or 40 mg/kg. In one embodiment, an exemplary dosage schedule includes a dose at, e.g., 25-40 mg/kg, administered once per every 3 weeks. In one embodiment, an exemplary dosage schedule includes a dose at, e.g., 5 mg/kg, 10 mg/kg, 15 mg/kg, 20 mg/kg, 25 mg/kg, 30 mg/kg or 40 mg/kg, administered once per every 3 weeks. Doses intermediate in the above ranges are also intended to be within the scope of the invention. In some methods, two or more monoclonal antibodies with different binding specificities are administered simultaneously, in which case the dosage of each antibody administered may fall within the ranges indicated.

It will be understood by one of skill in the art that the exemplary doses as described herein can also be expressed as amount (e.g., in milligrams) of binding molecule administered per body surface area (BSA) of the subject, e.g., mg/m². The body surface area of a subject can be calculated according to methods known in the art. For example, the body surface area may be calculated using the Mosteller formula as follows:

BSA(m²)=([Height(cm)×Weight(kg)]/3600)^{1/2}

Other methods for calculating the BSA are also known in the art, including the DuBois and DuBois formula, the Haycock formula, the Gehan and George formula and the Boyd formula. Doses expressed in mg/kg in any given species may be converted to the equivalent dose in mg/m² by multiplying the dose by the appropriate “Surface Area to Weight Ratio” (km) for the species. The km factors for representative species include the following: 3.0 kg/m² for mouse; 5.9 kg/m² for rat, 12 kg/m² for monkey, 20 kg/m² for dog, 25 kg/m² for a human child and 37 kg/m² for a human adult (see, e.g., Freireich, E J et al. Cancer Chemother. Rep. 1966 50(4):219-244). Thus, for example, in adult humans, a dose of 100 mg/kg is equivalent to 100 mg/kg×37 kg/m²=3700 mg/m².

In one embodiment, binding molecules of the invention can be administered on multiple occasions. Intervals between single dosages can be, e.g., daily, weekly, biweekly, once every three weeks, monthly or yearly. Intervals can also be irregular as indicated by measuring blood levels of polypeptide or target molecule in the patient. In some methods, dosage is adjusted to achieve a certain plasma binding molecule or toxin concentration, e.g., 1-1000 μg/ml or 25-300 μg/ml. Alternatively, binding molecules can be administered as a sustained release formulation, in which case less frequent administration is required. Dosage and frequency vary depending on the half-life of the antibody in the patient. In general, humanized antibodies show the longest half-life, followed by chimeric antibodies and nonhuman antibodies. In one embodiment, the half-life of humanized antibodies of the invention (e.g., conjugated humanized antibodies, e.g., B3F6.1-DM4) is about 100 hours, or about 4.2 days. In one embodiment, the binding molecules of the invention can be administered once or multiple times in unconjugated form. In another embodiment, the polypeptides of the invention can be administered once or multiple times in conjugated form. In still another embodiment, the binding molecules of the invention can be administered once or multiple times in unconjugated form, then in conjugated form, or vise versa.

The dosage and frequency of administration can vary, e.g., depending on whether the treatment is for an early or late stage malignancy. In one application, compositions containing the present antibodies or a cocktail thereof are administered at lower doses. In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In other therapeutic applications, a relatively high dosage (e.g., from about 1 to 400 mg/kg of binding molecule, e.g., antibody per dose, with dosages of from 5 to 25 mg/kg being more commonly used for radioimmunoconjugates and higher doses, e.g., 5-50 mg/kg, for cytotoxin-drug conjugated molecules) at relatively short intervals is sometimes required until progression of the disease is reduced or terminated, and preferably until the patient shows partial or complete amelioration of symptoms of disease. Thereafter, the patient may be administered a lower dose regime.

In one embodiment, binding molecules of the invention (e.g., a humanized anti-Cripto antibody conjugated to a maytansinoid, such as DM4) can be administered to patients having an established tumor, e.g., a tumor of relatively large size. In one embodiment, binding molecules of the invention (e.g., a humanized anti-Cripto antibody conjugated to a maytansinoid, such as DM4) can be administered to patients having an advanced tumor, e.g., a recurrent tumor or resistant tumor, e.g., a tumor that is unresponsive to other treatments. In such therapeutic applications, a single dosage (e.g., from about 1-100 mg/kg, 5-50 mg/kg, more preferably from about 10-40 mg/kg, and even more preferably from 15-30 mg/kg, including intermediate dosages to those above, including, e.g., 15 mg/kg, 20 mg/kg, 25 mg/kg, and 30 mg/kg) can be administered.

In one embodiment, a single dose of a binding molecule of the invention produces an anti-tumor response which is sustained for at least one week, two weeks, three weeks, four weeks, five weeks, six weeks, 3 months, 6 months or more. In one embodiment, multiple doses of a binding molecule of the invention, e.g., a biweekly dose or one dose of every three weeks, produce an anti-tumor response which is sustained for at least one week, two weeks, three weeks, four weeks, five weeks, six weeks, 3 months, 6 months or more.

In one embodiment, a subject can be treated with a nucleic acid molecule encoding a binding molecule of the invention (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

A. Administration in Combination with Other Agents

Agents of the invention can optionally be administered in combination with other agents that are effective in treating the disorder or condition in need of treatment (e.g., prophylactic or therapeutic). Preferred additional agents are those which are art recognized and are standardly administered for a particular disorder.

Effective single treatment dosages (i.e., therapeutically effective amounts) of ⁹⁰Y-labeled polypeptides of the invention range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-à-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, ⁴⁷Sc, ¹⁰⁵Rh, ¹⁰⁹Pd, ¹⁵³Sm, ¹⁸⁸Re, ¹⁹⁹Au, ²²⁵Ac, ²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not the binding molecules of the invention are used in a conjugated or unconjugated form, it will be appreciated that a major advantage of the present invention is the ability to use these polypeptides in myelosuppressed patients, especially those who are undergoing, or have undergone, adjunct therapies such as radiotherapy or chemotherapy. In other preferred embodiments, the polypeptides (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies and one or more chemotherapeutic agents. Particularly preferred embodiments of this aspect of the invention will comprise the administration of a toxin conjugated binding molecule, e.g., conjugated to a maytansinoid such as a D4 maytansinoid.

While the binding molecules may be administered as described immediately above, it must be emphasized that in other embodiments conjugated and unconjugated polypeptides may be administered to otherwise healthy patients as a first line therapeutic agent. In such embodiments the polypeptides may be administered to patients having normal or average red marrow reserves and/or to patients that have not, and are not, undergoing adjunct therapies such as external beam radiation or chemotherapy.

However, as discussed above, selected embodiments of the invention comprise the administration of polypeptides to myelosuppressed patients or in combination or conjunction with one or more adjunct therapies such as radiotherapy or chemotherapy (i.e. a combined therapeutic regimen). As used herein, the administration of polypeptides in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated polypeptides could be administered intravenously followed by tumor localized external beam radiation. In yet other embodiments, the polypeptide may be administered concurrently with one or more selected chemotherapeutic agents in a single office visit. A skilled artisan (e.g. an experienced oncologist) would readily be able to discern effective combined therapeutic regimens without undue experimentation based on the selected adjunct therapy and the teachings of the instant specification.

In this regard it will be appreciated that the combination of the polypeptide (either conjugated or unconjugated) and the chemotherapeutic agent may be administered in any order and within any time frame that provides a therapeutic benefit to the patient. That is, the chemotherapeutic agent and polypeptide may be administered in any order or concurrently. In selected embodiments the polypeptides of the present invention will be administered to patients that have previously undergone chemotherapy. In yet other embodiments, the polypeptides and the chemotherapeutic treatment will be administered substantially simultaneously or concurrently. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the polypeptide will be administered within 4, 3, 2 or 1 week of any chemotherapeutic agent or treatment. In yet other embodiments the polypeptide will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppresion and one can easily measure the extent to which myelosuppresion is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

In one embodiment, the binding molecules of the invention (either conjugated or unconjugated) are administered in combination with an additional agent, e.g., a chemotherapeutic agent, e.g., an antimetabolite. In one embodiment, the binding molecule functions or acts better in combination with the additional agent (e.g., additively or synergistically) than it acts alone to inhibit growth of tumor cells. In this embodiment, the administration of the binding molecule in combination with the additional agent, e.g., chemotherapeutic agent, inhibits growth of tumor cells more effectively than administration of either the binding molecule or additional agent, e.g., chemotherapeutic agent, alone. Preferably, the combination therapy inhibits tumor growth by, e.g, 50%, 60%, 70%, 80%, 90%, 95% or more. Those skilled in the art will readily be able to determine standard dosages and scheduling appropriate for these regimens, depending on the additional agent employed. In one embodiment, the additional agent is an antimetabolite, e.g., a pyrimidine analog, e.g., 5′-fluorouracil. In one embodiment, the additional agent is a pyrimidine analog, e.g., 5′fluorouracil. In one embodiment, the additional agent is a pyrimidine analog, e.g., 5′-fluorouracil and the binding molecule (e.g., B3F6.1) is conjugated to a toxin, such as a maytansinoid, e.g., DM4. In one embodiment, the 5′-fluorouracil is administered at a dose of 30 mg/kg. In one embodiment, the 5′-fluorouracil is administered at a maximum tolerated dose. In one embodiment, the 5′-fluorouracil is administered at a dose of 30 mg/kg and the binding molecule, e.g., humanized anti-Cripto antibody conjugated to a maytansinoid (e.g., DM4), is administered at a dose of 15 mg/kg. Combined administration of a binding molecule of the invention (e.g., a binding molecule of the invention conjugated to a toxin, such as a maytansinoid, e.g., DM4) with an antimetabolite, such as a pyrimidine analog, e.g., 5′-fluorouracil is particularly useful in the treatment of colon cancer. In a preferred embodiment, a humanized anti-Cripto antibody conjugated to a maytansinoid (e.g., DM4) is administered in combination with 5′ fluorouracil for the treatment of colon cancer.

More specifically conjugated or unconjugated polypeptides of the present invention may be used to effectively treat patients having ANCs lower than about 2000/mm³ or platelet counts lower than about 150,000/mm³. More preferably the polypeptides of the present invention may be used to treat patients having ANCs of less than about 1500/mm³, less than about 1000/mm³ or even more preferably less than about 500/mm³. Similarly, the polypeptides of the present invention may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, the disclosed polypeptides may be used to treat disorders in patients exhibiting myelosuppression regardless of the cause.

In this regard it will further be appreciated that the polypeptides of the instant invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other preferred embodiments the radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include alkylating agents, vinca alkaloids (e.g., vincristine and vinblastine), procarbazine, methotrexate, prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more polypeptides of the invention as described herein.

Additional regimens that are useful in the context of the present invention include use of antimetabolites. The term “antimetabolite,” as used herein, includes, but is not limited to, folic acid analogs, purine analogs and pyrimidine analogs. Nonlimiting examples of folic acid analogs include, e.g., methotrexate, pemetrexed, and raltitrexed. Nonlimiting examples of purine analogs include, e.g., azathioprine, 6-mercaptopurine, mercaptopurine, thioguanine, fludarabine, pentostatin and cladribine. Nonlimiting examples of pyrimidine analogs include, e.g., 5′-fluorouracil, floxuridine and cytosine arabinoside. A preferred antimetabolite of the invention is a pyrimidine analog. A particularly preferred antimetabolite of the invention is 5′-fluorouracil. Those skilled in the art will readily be able to determine standard dosages and scheduling for each of these regimens.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade NHL, who fail to achieve remission or relapse, salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the polypeptides of the instant invention may vary by subject or may be administered according to what is known in the art. See for example, Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996). In one embodiment, the chemotherapeutic agent to be used in combination with the polypeptides of the instant invention may be administered at their maximul tolerated dose.

In one embodiment, a binding molecule of the invention may be administered to a subject who has undergone, is undergoing, or will undergo a surgical procedure, e.g., to remove a primary tumor, a metastasis or precancerous growth or tissue as a preventative therapy.

In another embodiment, a binding molecule of the invention is administered in conjunction with a biologic. Biologics useful in the treatment of cancers are known in the art and a binding molecule of the invention may be administered, for example, in conjunction with such known biologics.

For example, the FDA has approved the following biologics for the treatment of breast cancer: Herceptin® (trastuzumab, Genentech Inc., South San Francisco, Calif.; a humanized monoclonal antibody that has antitumor activity in HER2-positive breast cancer); Faslodex® (fulvestrant, AstraZeneca Pharmaceuticals, LP, Wilmington, Del.; an estrogen-receptor antagonist used to treat breast cancer); Arimidex® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); Femara® (letrozole, Novartis Pharmaceuticals, East Hanover, N.J.; a nonsteroidal aromatase inhibitor approved by the FDA to treat breast cancer); and Nolvadex® (tamoxifen, AstraZeneca Pharmaceuticals, LP; a nonsteroidal antiestrogen approved by the FDA to treat breast cancer). Other biologics with which the binding molecules of the invention may be combined include: Avastin™ (bevacizumab, Genentech Inc.; the first FDA-approved therapy designed to inhibit angiogenesis); and Zevalin® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: Avastin™; Erbitux™ (cetuximab, ImClone Systems Inc., New York, N.Y., and Bristol-Myers Squibb, New York, N.Y.; is a monoclonal antibody directed against the epidermal growth factor receptor (EGFR)); Gleevec® (imatinib mesylate; a protein kinase inhibitor); and Ergamisol® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For use in treatment of Non-Hodgkin's Lymphomas currently approved therapies include: Bexxar® (tositumomab and iodine 1-131 tositumomab, GlaxoSmithKline, Research Triangle Park, N.C.; a multi-step treatment involving a mouse monoclonal antibody (tositumomab) linked to a radioactive molecule (iodine I-131)); Intron® A (interferon alfa-2b, Schering Corporation, Kenilworth, N.J.; a type of interferon approved for the treatment of follicular non-Hodgkin's lymphoma in conjunction with anthracycline-containing combination chemotherapy (e.g., cyclophosphamide, doxorubicin, vincristine, and prednisone [CHOP])); Rituxan® (rituximab, Genentech Inc., South San Francisco, Calif., and Biogen Idec, Cambridge, Mass.; a monoclonal antibody approved for the treatment of non-Hodgkin's lymphoma; Ontak® (denileukin diftitox, Ligand Pharmaceuticals Inc., San Diego, Calif.; a fusion protein consisting of a fragment of diphtheria toxin genetically fused to interleukin-2); and Zevalin® (ibritumomab tiuxetan, Biogen Idec; a radiolaebeled monoclonal antibody approved by the FDA for the treatment of B-cell non-Hodgkin's lymphomas).

For treatment of Leukemia, exemplary biologics which may be used in combination with the binding molecules of the invention include Gleevec®; Campath®-1H (alemtuzumab, Berlex Laboratories, Richmond, Calif.; a type of monoclonal antibody used in the treatment of chronic Lymphocytic leukemia). In addition, Genasense (oblimersen, Genta Corporation, Berkley Heights, N.J.; a BCL-2 antisense therapy under development to treat leukemia may be used (e.g., alone or in combination with one or more chemotherapy drugs, such as fludarabine and cyclophosphamide) may be administered with the claimed binding molecules.

For the treatment of lung cancer, exemplary biologics include Tarceva™ (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include Velcade® Velcade (bortezomib, Millennium Pharmaceuticals, Cambridge Mass.; a proteasome inhibitor). Additional biologics include Thalidomid® (thalidomide, Clegene Corporation, Warren, N.J.; an immunomodulatory agent and appears to have multiple actions, including the ability to inhibit the growth and survival of myeloma cells and antiangiogenesis).

Other exemplary biologics include the MOAB IMC-C225, developed by ImClone Systems, Inc., New York, N.Y.

In addition, the claimed binding molecules may be administered in conjunction with vaccines or other agents (e.g., cytokines) to modulate anti-cancer immune responses. For example, Melacine® (Corixa Corporation, Seattle, Wash.) is an allogeneic tumor vaccine that has been reported to have promising results in the treatment of T3N0M0 resected melanoma. GMK® (Progenics Pharmaceutical, Inc., Tarrytown, N.Y.) is a ganglioside antigen administered as an adjuvant phase III agent in patients who are at high risk for melanoma recurrence. Anti-gastrin Therapeutic Vaccine® (Aphton Corporation, Miami, Fla.) neutralizes hormones G 17 and glyextened and is in phase III clinical trials for patients with colorectal, pancreatic, and stomach cancers. CeaVac® (Titan Pharmaceuticals, Inc., South San Francisco, Calif.) is an anti-idiotype antibody vaccine being studied in colorectal cancer. Finally, Theratope® (Biomira Inc., Edmonton, Alberta, Canada) is a synthetic carbohydrate therapeutic vaccine being investigated as a phase III agent in patients with metastatic breast cancer (Pharmaceutical Research and Manufacturers of America, 2000).

In another embodiment, a binding molecule of the invention may be administered in conjunction with an anti-angiogenesis agent, e.g., Endostatin (an endogenous, tumor-derived, endothelial-specific inhibitor that halts microvascular endothelial cell production); anti-VEGF antibody; thalidomide; or matrix metalloproteinase inhibitors inhibit the synthesis and degradation of the basement membrane of blood vessels).

As previously discussed, the polypeptides of the present invention, immunoreactive fragments or recombinants thereof may be administered in a pharmaceutically effective amount for the in vivo treatment of mammalian disorders. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For example, pharmaceutical compositions in accordance with the present invention can comprise succinic acid as the pH buffer, any one or all of L-glycine, glycerol and polysorbate 80 as stabilizers, WFI as solvent and sodium hydroxide for pH adjustment. In a preferred embodiment, the pharmaceutical compositions of the invention comprise 10 mM sodium succinate, 120 mM L-glycine, 120 mM glycerol, 0.01% Polysorbate 80 at pH 5.0. Preferably, the anti-Cripto binding molecule, e.g, humanized anti-Cripto antibody-maytansinoid conjugate, e.g., B3F6.1-DM4, is present in the pharmaceutical formulation at a concentration of between about 1 mg/ml and 10 mg/ml, and preferably at 1, 2, 3, 4, 5, 6, 7, 8, 9 or 10 mg/ml. In a preferred embodiment, the anti-Cripto binding molecule, e.g, humanized anti-Cripto antibody-maytansinoid conjugate, e.g., B3F6.1-DM4, is present in the pharmaceutical formulation at a concentration of 5 mg/ml. In one embodiment such formulations comprise anti-Cripto antibodies having an average of 3.5 DM4 molecules per molecule of antibody. Pharmaceutical formulations of the invention will be stable at temperatures between 2° and 8° C., e.g., at 5° C., for at least 12 months, preferably for at least 24 months and most preferably for at least 36 months. Pharmaceutical formulations of the invention will be stable at accelerated temperatures, such as at 25° C., for at least 3 months, preferably at least 6 months and more preferably at least 12 months.

For the purposes of the instant application, a pharmaceutically effective amount of the polypeptide, immunoreactive fragment or recombinant thereof, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the polypeptide will be preferably be capable of interacting with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the polypeptide.

In keeping with the scope of the present disclosure, the polypeptides of the invention may be administered to a human or other animal in accordance with the aforementioned methods of treatment in an amount sufficient to produce a therapeutic or prophylactic effect. The polypeptides of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of polypeptides according to the present invention may prove to be particularly effective.

### VII. Methods of Use

The molecules of the invention can be used primarily for therapeutic purposes. Preferred embodiments of the present invention provide compounds, compositions, kits and methods for the diagnosis and/or treatment of disorders, e.g., neoplastic disorders in a mammalian subject in need of such treatment. Preferably, the subject is a human.

The polypeptides of the instant invention will be useful in a number of different applications. For example, in one embodiment, the subject binding molecules may be used in an assay to detect Cripto in vitro, e.g., using an ELISA assay. Exemplary assays are known in the art, see, e.g., United States Application Number 20040077025.

In another embodiment, the subject binding molecules are useful for detecting the presence of Cripto bearing cells using imaging technology. For such applications, it may be desirable to conjugate the binding molecule to a detectable moiety, e.g., a radiolabel, as described further below.

In another embodiment, the subject binding molecules are useful for reducing or eliminating cells bearing target (e.g., an epitope of Cripto) recognized by a binding molecule of the invention. In another embodiment, the subject binding molecules are effective in reducing the concentration of or eliminating soluble target molecules in the circulation

In one embodiment, a binding molecule of the invention reduces tumor size, inhibits tumor growth and/or prolongs the survival time of a tumor-bearing subject. Accordingly, this invention also relates to a method of treating tumors in a human or other animal by administering to such human or animal an effective, non-toxic amount of polypeptide. One skilled in the art would be able, by routine experimentation, to determine what an effective, non-toxic amount of polypeptide would be for the purpose of treating malignancies. For example, a therapeutically active amount of a polypeptide may vary according to factors such as the disease stage (e.g., stage I versus stage IV), age, sex, medical complications (e.g., immunosuppressed conditions or diseases) and weight of the subject, and the ability of the antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 120 milligrams per kilogram body weight per day, preferably from about 0.1 to 100 milligrams per kilogram body weight per day and more preferably from about 0.5 to 50 milligrams per kilogram body weight per day.

For purposes of clarification “mammal” refers to any animal classified as a mammal, including humans, domestic and farm animals, and zoo, sports, or pet animals, such as dogs, horses, cats, cows, etc. Preferably, the mammal is human. “Treatment” refers to both therapeutic treatment and prophylactic or preventative measures. Those in need of treatment include those already with the disease or disorder as well as those in which the disease or disorder is to be prevented. Hence, the mammal may have been diagnosed as having the disease or disorder or may be predisposed or susceptible to the disease.

In general, the disclosed invention may be used to therapeutically treat any neoplasm comprising a marker that allows for the targeting of the cancerous cells by the binding molecule. In a preferred embodiment, the binding molecules of the invention are used to treat solid tumors. Exemplary cancers that may be treated include, but are not limited to, prostate, gastric carcinomas such as colon and colorectal, skin, breast, ovarian, endometrial, lung, non-small cell lung, and pancreatic cancer. In another embodiment, the antibodies of the instant invention may be used to treat Kaposi's sarcoma, CNS neoplasias (capillary hemangioblastomas, meningiomas and cerebral metastases), melanoma, gastrointestinal and renal sarcomas, rhabdomyosarcoma, glioblastoma (preferably glioblastoma multiforme), leiomyosarcoma, retinoblastoma, papillary cystadenocarcinoma of the ovary, Wilm's tumor or small cell lung carcinoma. It will be appreciated that appropriate polypeptides may be derived for tumor associated molecules related to each of the forgoing neoplasias without undue experimentation in view of the instant disclosure.

Exemplary hematologic malignancies that are amenable to treatment with the disclosed invention include Hodgkins and non-Hodgkins lymphoma as well as leukemias, including ALL-L3 (Burkitt's type leukemia), chronic lymphocytic leukemia (CLL) and monocytic cell leukemias. It will be appreciated that the compounds and methods of the present invention are particularly effective in treating a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention. In addition to the aforementioned neoplastic disorders, it will be appreciated that the disclosed invention may advantageously be used to treat additional malignancies bearing compatible tumor associated molecules.

In one embodiment of the invention, molecules are provided which are capable of binding specifically to Cripto and which inhibit growth of tumor cells in a patient, especially where the tumor growth is mediated by the loss or decrease of Activin B signaling. In certain embodiments, the tumor cells are brain, head, neck, prostate, breast, testicular, colon, colorectal, lung, non-small cell lung, ovary, bladder, uterine, endometrium, cervical, pancreatic and stomach tumor cells. In other embodiments, a binding molecule of the invention binds specifically to Cripto and inhibits growth of tumor cells which overexpress Cripto. In one embodiment, the tumor cells are cell lines which overexpress Cripto, such as cell lines derived from brain, breast, testicular, colon, colorectal, lung, non-small cell lung, ovary, bladder, uterine, endometrium, cervical, pancreatic and stomach cancers.

This invention is further illustrated by the following examples which should not be construed as limiting. The contents of all references, patents and published patent applications cited throughout this application are incorporated herein by reference.

### EXAMPLES

### Example 1

### Humanized B3F6 Antibody Conjugated to a Toxin is Effective in Inhibiting the Growth of Human Colon Tumor Cells when Administered in a Single or Two Biweekly Doses in an In Vivo Model

The following materials and methods were used in this example:

### Mice

Two hundred eighteen (218) female SCID beige (C.B.-17/IcrHsd-Prkcd Lyst Skid Beige) mice (Harlan Sprague Dawley, Madison, Wis.) were started on the study at six to seven weeks of age. Animals were acclimated to the laboratory for at least two days prior to implantation of the tumor. Housing was in ventilated cage racks, and food and water were allowed ad libitum.

### Tumor Model

CT-3 tumor fragments from a primary human colon tumor were originally obtained from Sera Care, Inc (Oceanside, Calif.) (sent by Peter Chu, Biogen Idec, San Diego). A serially transplanted in-vivo xenograft line was established at Biogen Idec, Inc. and fragments from the third xenograft generation were cryopreserved. These cryopreserved fragments (Biogen Idec cryo reg #0226) were thawed and serially passaged SC in vivo for 3-5 generations in female SCID beige mice prior to implantation for this study. Bacterial cultures were performed on samples of the tumor tissue that was implanted into the mice. Bacteriology cultures were negative for bacterial contamination at both 24 and 48 hours post implant.

On Day −1, the mice were implanted with BioMedics animal ID chips (Model IMI-1000; Seaford, Del.) SC on the left flank. On Day 0, tumors from twelve donor animals were harvested, debrided of necrotic tissue, minced, and a 3 mm³ fragments of the CT-3 tumors were implanted SC into the right flank area of each mouse. Tumor size and body weight measurements were recorded at least twice weekly beginning on Day 5. When the tumors measured a minimum of 100 mg (Day 15), mice were randomized to treatment and control groups (see Table 1) based on tumor size and excluding tumors with non-progressive growth.

TABLE 1
Control and Test Treatment Groups

| | | Equivalent | | | |
|---|---|---|---|---|---|
| | | dose of | | | |
| | Dose/ | maytansine | | | # of |
| Agent | injection | (μg/kg) | Route | Schedule | mice |
| Vehicle control | 10 ml/kg | 0 | IV^{a} | Single dose | 16 |
| B3F6.1-DM4 | 25 mg/kg | 353 | IV | Day 14 | 8 |
| B3F6.1-DM4 | 40 mg/kg | 564 | IV | Day 14 | 8 |
| B3F6.1-DM4 | 25 mg/kg | 353 | IV | q14dx2 | 8 |
| B3F6.1-DM4 | 40 mg/kg | 564 | IV | q14dx2 | 8 |

^{a}intravenous

### Test Articles and Positive Chemotherapeutic Agent

Maytansin DM4 conjugations (2000-112, 5.9 mg/ml) were prepared at ImmunoGen, Inc (Cambridge, Mass.) with ImmunoGen's Tumor Activated Prodrug (TAP) technology. Clinical grade Adrucil (5-fluorouracil, NDC 0703-3015-11) was obtained from Sicor Pharmaceuticals (Lot No. 06A625, exp. July 2007).

### Study Groups and Treatment Regimens

Study groups and treatment regimens are described in Table 1. The vehicle control ((10 mM citrate buffer, pH 5.5, 135 mM sodium chloride)) was administered IV as a single dose at Day 15. B3F6.1-DM4 at 25 mg/kg or 40 mg/kg was administered IV as a single dose at Day 15. B3F6.1-DM4 at 25 mg/kg or 40 mg/kg was alternatively administered IV q14d×2 (two doses). All treatments commenced on Day 15.

### Evaluation of Anticancer Activity

Tumor measurements were determined using digital calipers. Body weights and tumor size measurements were recorded on Day 5 and were continued twice weekly until the termination of the study. The formula to calculate volume for a prolate ellipsoid was used to estimate tumor volume (mm³) from two-dimensional tumor measurements: Tumor Volume (mm³)=(Length×Width²)÷2. Assuming unit density, volume was converted to weight (i.e., one mm³=one mg).

### Statistical Analysis

Student's t test was performed on mean tumor weights at the end of each study to determine whether there were any statistically significant differences between each treatment group and the vehicle control group.

There was a 95% tumor take-rate following the implantation, and mice within a tight range of tumor weight were selected to initiate the treatments. The tumor growth in the vehicle control group was well within the typical range we see with this model.

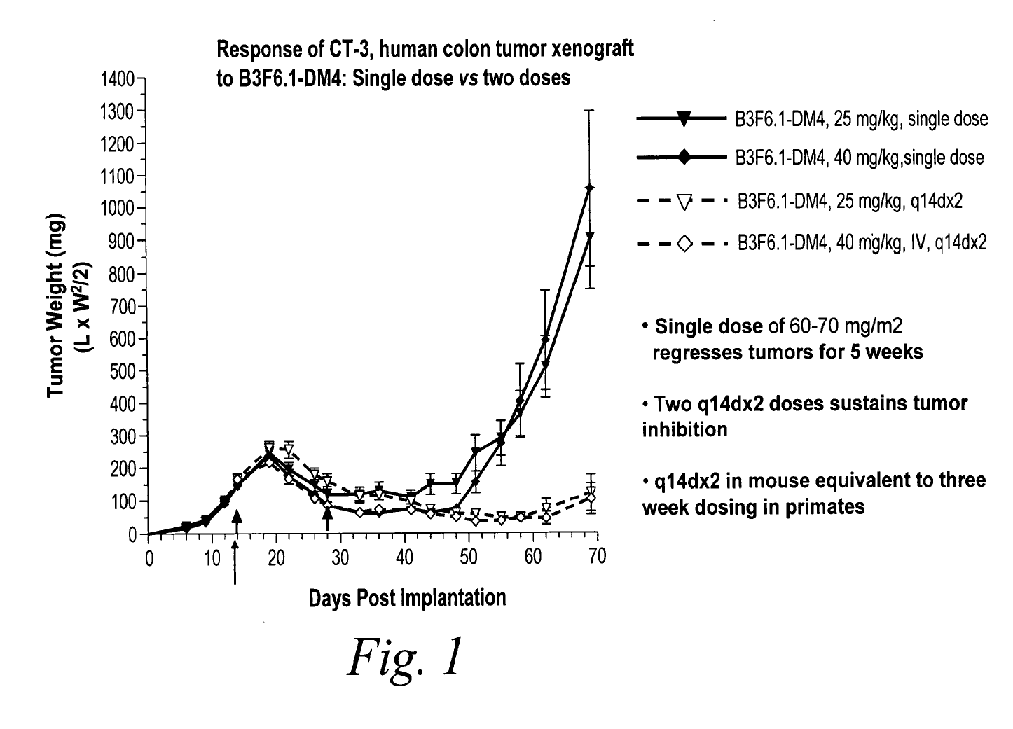

FIG. 1 shows the effect of a single dose (25 and 40 mg/kg/inj) or two doses (25 and 40 mg/kg/inj) of B3F6.1-DM4 dosed IV on various regimens on change in tumor weight in athymic nude mice bearing established CT-3 xenograft tumors. A single dose of B3F6.1-DM4 at 25 mg/kg/inj or at 40 mg/kg/inj dosed IV significantly inhibited tumor growth for up to 5 weeks (Day 49). The other cohort treated with B3F6.1-DM4 at 25 mg/kg/inj, dosed IV q14d×2 and at 40 mg/kg/inj, dosed IV q14d×2, showed significant inhibition of tumor growth throughout the study (8 weeks), until the study was terminated (Day 70). These results demonstrate that a single dose of 60-70 mg/m2 causes a regression of tumors for up to 5 weeks in this in vivo murine model. These results further demonstrate that two doses of B3F6.1-DM4 administered biweekly, i.e., q14d×2, sustains tumor inhibition in this in vivo murine model. A q14d×2 dose in mice is equivalent to a once every three week dosing in primates. These results thus indicate that an effective dose of B3F6.1-DMF in man includes a dosing regimen of administration once every 3 weeks.

### Example 2

### Humanized B3F6 Antibody is Effective in Inhibiting the Growth of Human Colon Tumor Cells Synergistically when Administered in Conjunction with a Chemotherapeutic Agent in an In Vivo Model.

### Mice

Two hundred eighteen (218) female SCID beige (C.B.-17/IcrHsd-Prkcd Lyst Skid Beige) mice (Harlan Sprague Dawley, Madison, Wis.) were started on the study at six to seven weeks of age. Animals were acclimated to the laboratory for at least two days prior to implantation of the tumor. Housing was in ventilated cage racks, and food and water were allowed ad libitum.

### Tumor Model

CT-3 tumor fragments from a primary human colon tumor were originally obtained from Sera Care, Inc (Oceanside, Calif.) (sent by Peter Chu, Biogen Idec, San Diego). A serially transplanted in-vivo xenograft line was established at Biogen Idec, Inc. and fragments from the third xenograft generation were cryopreserved. These cryopreserved fragments (Biogen Idec cryo reg #0226) were thawed and serially passaged SC in vivo for 3-5 generations in female SCID beige mice prior to implantation for this study. Bacterial cultures were performed on samples of the tumor tissue that was implanted into the mice. Bacteriology cultures were negative for bacterial contamination at both 24 and 48 hours post implant.

On Day −1, the mice were implanted with BioMedics animal ID chips (Model IMI-1000; Seaford, Del.) SC on the left flank. On Day 0, tumors from eight donor animals were harvested, debrided of necrotic tissue, minced, and a 3 mm³ fragments of the CT-3 tumors were implanted SC into the right flank area of each mouse. Tumor size and body weight measurements were recorded at least twice weekly beginning on Day 5. When the tumors measured a minimum of 100 mg (Day 15), mice were randomized to treatment and control groups (see Table 2) based on tumor size and excluding tumors with non-progressive growth.

TABLE 2
Control and Test Treatment Groups

| | | Equivalent | | | |
|---|---|---|---|---|---|
| | | dose of | | | |
| | Dose/ | maytansine | | | # of |
| Agent | injection | (μg/kg) | Route | Schedule | mice |
| Vehicle control | 10 ml/kg/inj | 0 | IV^{a} | Day 15 | 16 |
| B3F6.1-DM4 | 15 mg/kg | 212 | IV | Day 15 | 8 |
| 5-fluorouracil | 30 mg/kg | 0 | IV | Day 15 | 8 |
| B3F6.1-DM4 | 15 mg/kg | 212 | IV | Day 15 | 8 |
| 5-fluorouracil | 30 mg/kg | 0 | IV | Day 15 | |

^{a}intravenous

### Test Articles and Positive Chemotherapeutic Agent

Maytansin DM4 conjugations (2000-112, 5.9 mg/ml) were prepared at ImmunoGen, Inc (Cambridge, Mass.) with ImmunoGen's Tumor Activated Prodrug (TAP) technology. Clinical grade Adrucil (5-fluorouracil, NDC 0703-3015-11) was obtained from Sicor Pharmaceuticals (Lot No. 06A625, exp. July 2007).

### Study Groups and Treatment Regimens

Study groups and treatment regimens are described in Table 2. The vehicle control (citrate buffer) was administered IV as a single dose at 10 ml/kg at Day 15. B3F6.1-DM4 at 15 mg/kg/inj was administered IV as a single dose at Day 15. 5-Fluorouracil at 30 mg/kg was administered IV as a single dose at Day 15. In addition, B3F6.1-DM4 at 15 mg/kg/inj was administered IV as a single dose at Day 15 in combination with the administration of 5-fluorouracil at 30 mg/kg/inj, also administered IV as a single dose at Day 15.

### Evaluation of Anticancer Activity

Tumor measurements were determined using digital calipers. Body weights and tumor size measurements were recorded on Day 6 and were continued twice weekly until the termination of the study. The formula to calculate volume for a prolate ellipsoid was used to estimate tumor volume (mm³) from two-dimensional tumor measurements: Tumor Volume (mm³)=(Length×Width²)÷2. Assuming unit density, volume was converted to weight (i.e., one mm³=one mg).

### Statistical Analysis

Student's t test was performed on mean tumor weights at the end of each study to determine whether there were any statistically significant differences between each treatment group and the vehicle control group.

There was a 95% tumor take-rate following the implantation, and mice within a tight range of tumor weight were selected to initiate the treatments. The tumor growth in the vehicle control group was well within the typical range we see with this model.

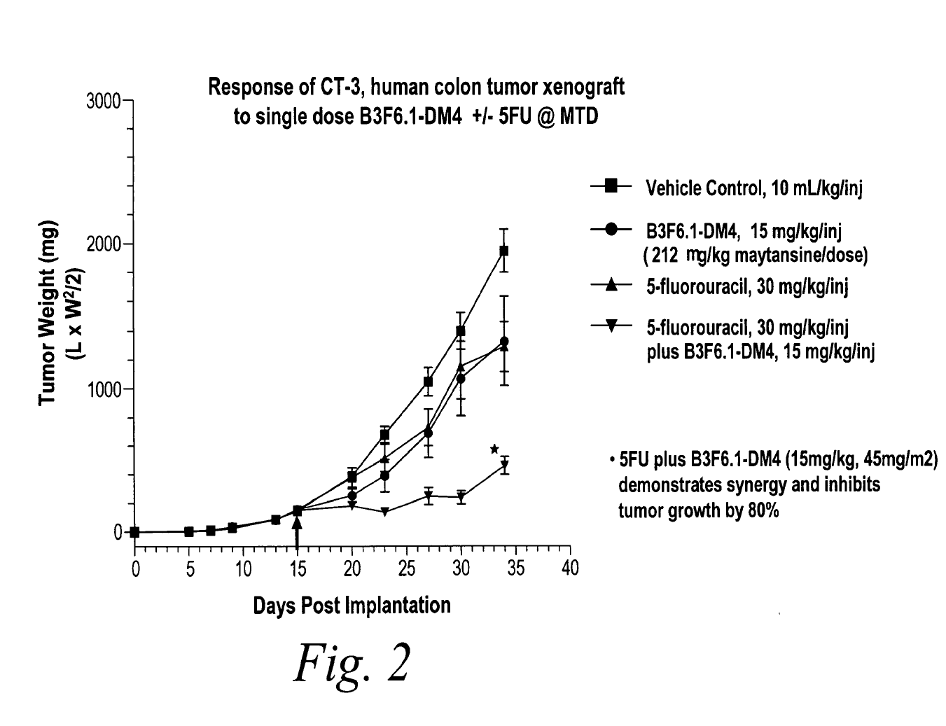

FIG. 2 shows the effect of a single dose (15 mg/kg/inj) of B3F6.1-DM4 or a single dose (of 30 mg/kg/inj) of 5-fluorouracil, each dosed IV, on change in tumor weight in athymic nude mice bearing established CT-3 xenograft tumors. A single dose of B3F6.1-DM4 at 15 mg/kg/inj or of 5-fluorouracil at 30 mg/kg/inj dosed IV significantly inhibited tumor growth throughout the study, until the study was terminated (Day 34). The other cohort treated with B3F6.1-DM4 at 15 mg/kg/inj in conjunction with 5-fluorouracil at 30 mg/kg/inj, showed a striking synergistic inhibition of tumor growth (inhibition by 80%) as compared to either B3F6.1-DM4 or 5-fluorouracil alone, throughout the study, until the study was terminated (Day 34). These results demonstrate that a single dose of 15 mg/kg (45 mg/m2) of B3F6.1-DM4 in combination with a single dose of an additional chemotherapeutic, e.g., 5-fluorouracil (30 mg/kg), results in a synergistic inhibition of tumor growth for up to 3 weeks in this in vivo murine model. These results indicate that a combination therapy including an anti-Cripto antibody, e.g., B3F6.1-DM4, in conjunction with an additional therapeutic, e.g., 5-fluorouracil, is an effective treatment for cancer, e.g., colon cancer, in man.

### Example 3

### Humanized B3F6 Antibody is Effective in Inhibiting the Growth of Large Human Colon Carcinoma Tumors in an In Vivo Model

The following materials and methods were used in this example:

### Mice

Two hundred ten (210) female SCID beige (C.B.-17/IcrHsd-Prkcd Lyst Skid Beige) mice (Harlan Sprague Dawley, Madison, Wis.) were started on the study at six to seven weeks of age. Animals were acclimated to the laboratory for at least two days prior to implantation of the tumor. Housing was in ventilated cage racks, and food and water were allowed ad libitum.

### Tumor Model

CT-3 tumor fragments from a primary human colon tumor were originally obtained from Sera Care, Inc (Oceanside, Calif.) (sent by Peter Chu, Biogen Idec, San Diego). A serially transplanted in-vivo xenograft line was established at Biogen Idec, Inc. and fragments from the third xenograft generation were cryopreserved. These cryopreserved fragments (Biogen Idec cryo reg #0239) were thawed and serially passaged SC in vivo for 2 generations in female SCID beige mice prior to implantation for this study. Bacterial cultures were performed on samples of the tumor tissue that was implanted into the mice. Bacteriology cultures were negative for bacterial contamination at both 24 and 48 hours post implant.

On Day −1, the mice were implanted with BioMedics animal ID chips (Model IMI-1000; Seaford, Del.) SC on the left flank. On Day 0, tumors from fourteen donor animals were harvested, debrided of necrotic tissue, minced, and a 3 mm³ fragments of the CT-3 tumors were implanted SC into the right flank area of each mouse. Tumor size and body weight measurements were recorded at least twice weekly beginning on Day 6. When the tumors measured a minimum of 80 mg (Day 18), mice were randomized to treatment and control groups (see Table 3) based on tumor size and excluding tumors with non-progressive growth.

TABLE 3
Control and Test Treatment Groups

| | | Equivalent | | | |
|---|---|---|---|---|---|
| | | dose of | | | |
| | Dose/ | maytansine | | | # of |
| Agent | injection | (μg/kg) | Route | Schedule | mice |
| Vehicle control: | 10 ml/kg, | 0 | IV^{a} | Single dose, | 13 |
| citrate buffer + | 10 ml/kg | | IP^{b} | Day 18 | |
| 0.9% saline | | | | q2dx6 | |
| | | | | (M, W, F) | |
| B3F6.1-DM4 | 15 mg/kg | 225 | IV | Day 30 | 8 |
| B3F6.1-DM4 | 25 mg/kg | 375 | IV | Day 30 | 8 |

^{a}intravenous
^{b}intraperitoneal

### Test Articles and Positive Chemotherapeutic Agent

Maytansin DM4 conjugations (2000-112, 5.9 mg/ml) were prepared at ImmunoGen, Inc (Cambridge, Mass.) with ImmunoGen's Tumor Activated Prodrug (TAP) technology. Clinical grade Adrucil (5-fluorouracil, NDC 0703-3015-11) was obtained from Sicor Pharmaceuticals (Lot No. 06A625, exp. July 2007).

### Study Groups and Treatment Regimens

Study groups and treatment regimens are described in Table 3. The vehicle control (10 mM citrate buffer, pH 5.5, 135 mM sodium chloride) was administered IV in a single dose at 10 ml/kg on day 18 and, in addition, 0.9% saline was administered intraperitoneally at a dose of 10 ml/kg, q2d×6 (M, W, and F) beginning on Day 18. B3F6.1-DM4 at 15 mg/kg or 25 mg/kg was administered IV as a single dose at day 30.

### Evaluation of Anticancer Activity

Tumor measurements were determined using digital calipers. Body weights and tumor size measurements were recorded on Day 6 and were continued twice weekly until the termination of the study. The formula to calculate volume for a prolate ellipsoid was used to estimate tumor volume (mm³) from two-dimensional tumor measurements: Tumor Volume (mm³)=(Length×Width²)÷2. Assuming unit density, volume was converted to weight (i.e., one mm³=one mg). The group was terminated on Day 39.

### Statistical Analysis

Student's t test was performed on mean tumor weights at the end of each study to determine whether there were any statistically significant differences between each treatment group and the vehicle control group.

There was a 95% tumor take-rate following the implantation, and mice within a tight range of tumor weight on Day 18 were selected to initiate the treatments. The tumor growth in the vehicle control group was well within the typical range we see with this model.

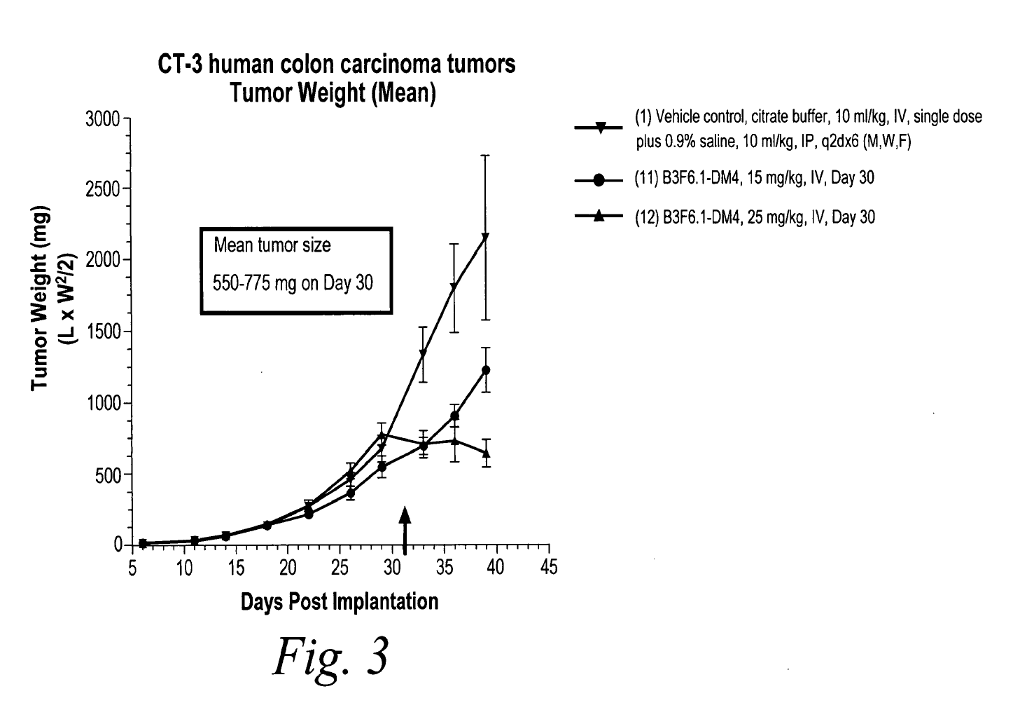

FIG. 3 shows the effect of a single dose (15 and 25 mg/kg/inj) of B3F6.1-DM4 dosed IV on change in tumor weight in athymic nude mice bearing large CT-3 xenograft tumors, e.g., tumors having a mean tumor weight of 550-775 mg. B3F6.1-DM4 at 15 mg/kg/inj or 25 mg/kg/inj dosed IV significantly inhibited tumor growth until the study was terminated (Day 39). These results demonstrate that a single dose of B3F6.1-DM4 is effective in inhibiting the growth of large tumors, e.g., human colon carcinoma tumors, in this in vivo murine model. These results indicate that administration of an anti-Cripto antibody, e.g., B3F6.1-DM4, even in a single dose, is an effective treatment for large, established tumors in man.

### Example 4

### Humanized B3F6 Antibody Linked to a Toxin Via Different Linkers are Effective in Inhibiting Growth of Human Testicular Carcinoma Cells

The following materials and methods were used in this example:

### Mice

Female SCID beige (C.B.-17/IcrHsd-Prkcd Lyst Skid Beige) mice (Harlan Sprague Dawley, Madison, Wis.) were started on the study at six to seven weeks of age. Animals were acclimated to the laboratory for at least two days prior to implantation of the tumor. Housing was in ventilated cage racks, and food and water were allowed ad libitum.

### Tumor Model

Human testicular carcinoma tumors were obtained from cryopreserved solid tumor fragments from a serially passaged in vivo donor line established at Biogen Idec. Tumor fragments were removed from cryopreservation and serially passaged SC in vivo for 3 generations in female athymic nude mice prior to implantation. Bacterial cultures were performed on samples of the tumor tissue that was implanted into the mice. Bacteriology cultures were negative for bacterial contamination at both 24 and 48 hours post implant.

On Day −1, the mice were implanted with BioMedics animal ID chips (Model IMI-1000; Seaford, Del.) SC on the left flank. On Day 0, tumors from donor animals were harvested, debrided of necrotic tissue, minced, and a 3 mm³ fragments of the CT-3 tumors were implanted SC into the right flank area of each mouse. Tumor size and body weight measurements were recorded at least twice weekly beginning on Day 5. When the tumors measured a minimum of 100 mg, mice were randomized to treatment and control groups (see Table 4) based on tumor size and excluding tumors with non-progressive growth.

TABLE 4
Control and Test Treatment Groups

| | | Equivalent | | |
|---|---|---|---|---|
| | | dose of | | |
| | Dose/ | maytansine | | |
| Agent | injection | (μg/kg) | Route | Schedule |
| Vehicle control | 10 ml./kg | 0 | IV^{a} | Day 14 |
| Cis-platinum | 2 mg/kg | 0 | IP^{b} | q2d6 |
| B3F6.1-SMCC-DM1 | 5 mg/kg | | IV | Day 14 |
| B3F6.1-SMMC-DM1 | 10 mg/kg | | IV | Day 14 |
| B3F6.1-SMMC-DM1 | 15 mg/kg | | IV | Day 14 |
| B3F6.1-SPDB-DM4 | 5 mg/kg | | IV | Day 14 |
| B3F6.1-SPDB-DM4 | 10 mg/kg | | IV | Day 14 |
| B3F6.1-SPDB-DM4 | 15 mg/kg | | IV | Day 14 |

^{a}intravenous
^{b}intraperitoneal

### Test Articles and Positive Chemotherapeutic Agent

Maytansin DM4 conjugations (2000-112, 5.9 mg/ml) were prepared at ImmunoGen, Inc (Cambridge, Mass.) with ImmunoGen's Tumor Activated Prodrug (TAP) technology. Clinical grade Adrucil (5-fluorouracil, NDC 0703-3015-11) was obtained from Sicor Pharmaceuticals (Lot No. 06A625, exp. July 2007).

### Study Groups and Treatment Regimens

Study groups and treatment regimens are described in Table 4. The vehicle control (10 mM citrate buffer, pH 5.5, 135 mM sodium chloride) was administered IV as a single dose at Day 14. B3F6.1-SMCC-DM1 at 5 mg/kg, 10 mg/kg or 15 mg/kg was administered IV as a single dose at Day 14. B3F6.1-SPDB-DM4 at 5 mg/kg, 10 mg/kg or 15 mg/kg was administered IV as a single dose at Day 14. Cis-platinum at 2 mg/kg was administered IP at q2d×6, beginning at Day 14.

### Evaluation of Anticancer Activity

Tumor measurements were determined using digital calipers. Body weights and tumor size measurements were recorded on Day 0 and were continued twice weekly until the termination of the study. The formula to calculate volume for a prolate ellipsoid was used to estimate tumor volume (mm³) from two-dimensional tumor measurements: Tumor Volume (mm³)=(Length×Width²)÷2. Assuming unit density, volume was converted to weight (i.e., one mm³=one mg).

### Statistical Analysis

Student's t test was performed on mean tumor weights at the end of each study to determine whether there were any statistically significant differences between each treatment group and the vehicle control group.

There was a 95% tumor take-rate following the implantation, and mice within a tight range of size were selected to initiate the treatments. The tumor growth in the vehicle control group was well within the typical range we see with this model.

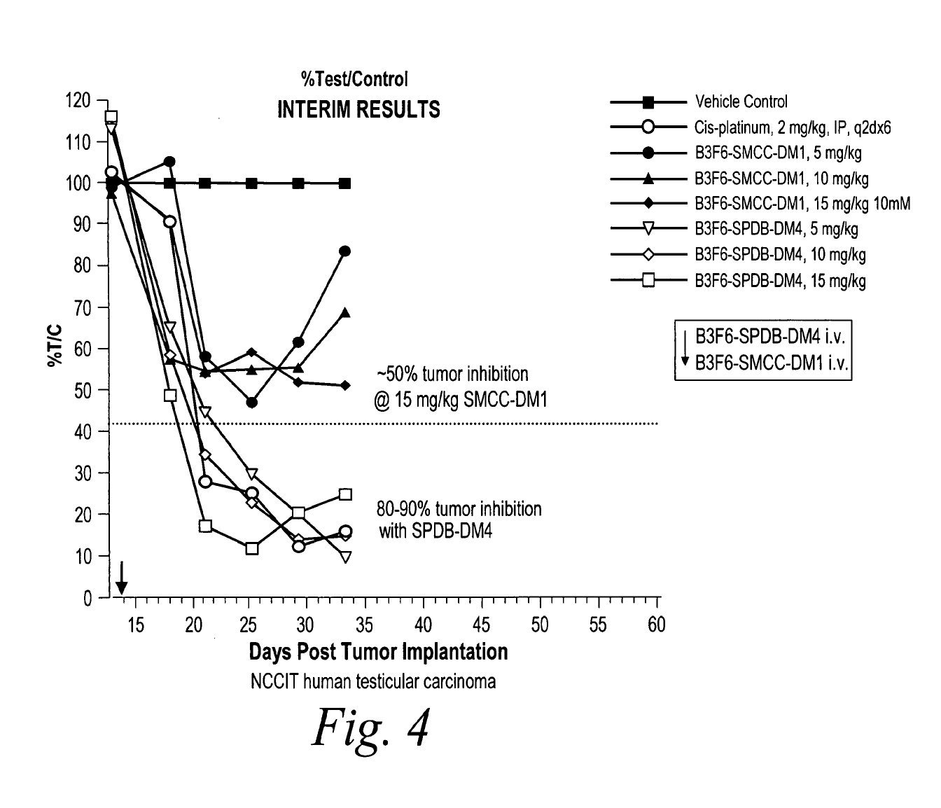

FIG. 4 shows the effect of a single dose (5, 10 and 15 mg/kg/inj) of B3F6.1-SMCC-DM1 or a single dose (5, 10 and 15 mg/kg/inj) of B3F6.1-SPDB-DM4 dosed IV on change in tumor weight in athymic nude mice bearing established human testicular xenograft tumors. A single dose of B3F6.1-SMCC-DM1 at 5 mg/kg, 10 mg/kg or 15 mg/kg dosed IV at Day 14 significantly inhibited tumor growth (approximately 50% tumor inhibition) throughout the study, until the study ws terminated (Day 34). The other cohort treated with B3F6.1-SPDB-DM4 at 5, 10 and 15 mg/kg/inj, dosed IV at Day 14 showed a striking, significant inhibition of tumor growth (approximately 80-90% tumor inhibition) throughout the study, until the study was terminated (Day 34). These results demonstrate that a single dose of B3F6.1-SMCC-DM1 at 5-15 mg/kg causes inhibition of tumor growth for up to 3 weeks in this in vivo murine model. These results further demonstrate that a single dose of B3F6.1-SPDB-DM4 at 5-15 mg/kg causes striking inhibition of tumor growth for up to 3 weeks in this in vivo murine model. A q14d×2 dose in mice is equivalent to a once every three week dosing in primates.

In this example, the various linker-maytansin conjugates linked to the B3F6 antibody are released from the conjugated B3F6 antibody with different half-lives. In particular, the SPP-DM1 linker conjugate has a half life of approximately 24-48 hours in man, the SPDB-DM4 linker conjugate has a half life of approximately 5 days in man, and the SMCC-DM1 linker conjugate has a half life of approximately 6 days in man. The SPP and SPDB linkers produce metabolites that can re-enter neighboring tumor cells, producing a so-called “bystander” effect that can contribute to tumor cell killing. In contrast, SMCC-DM1 linker system does not produce a metabolite product that can re-enter neighboring tumor cells. The results presented in this Example indicate that the B3F6-SMCC-DM1 molecule comprising the SMCC-DM1 linker system is active in tumors, e.g., testicular carcinomas, which do not require the “bystander killing” activity. The results presented in this Example also indicate that the B3F6-SPDB-DM4 molecule comprising the SPDB-DM4 linker system is more effective in inhibiting tumor growth than the B3F6 conjugates comprising the SPP-DM1 or SMCC-DM1 linker systems.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of inhibiting growth of a tumor in a subject, comprising administering to the subject an effective dose of an anti-Cripto antibody conjugated to a maytansoid, wherein the anti-Cripto antibody conjugate is administered in a single dose, biweekly, or once every three weeks, thereby inhibiting growth of a tumor in a subject.

2. (canceled)

3. The method of claim 1, wherein the anti-Cripto antibody is a humanized anti-Cripto antibody.

4. (canceled)

5. The method of claim 1, wherein the maytansinoid is DM4.

6. The method of claim 5, wherein there is an average of 3.5 molecules of DM4 attached to one molecule of the antibody.

7. The method of claim 4, wherein the maytansoid is conjugated to the antibody via a heterobifunctional crosslinking agent.

8. The method of claim 7, wherein the heterobifunctional crosslinking agent is 4-(2-pyridyldithio)butanoic acid N-hydroxysuccinimide ester (SPDB).

9. The method of claim 1, wherein the subject is suffering from a cancer in an organ selected from the group consisting of brain, breast, testicular, colon, lung, ovary, bladder, uterine, cervical, pancreatic and stomach.

10. (canceled)

11. (canceled)

12. A method of inhibiting growth of a tumor in a subject, comprising administering to the subject an effective dose of an anti-Cripto antibody conjugated to a maytansoid and an additional chemotherapeutic agent, thereby inhibiting growth of a tumor in the subject.

13. The method of claim 12, wherein the anti-Cripto antibody conjugate and the chemotherapeutic agent act synergistically.

14. The method of claim 12, wherein the chemotherapaeutic agent is an antimetabolite.

15. The method of claim 14, wherein the antimetabolite is a pyrimidine analog.

16. The method of claim 15, wherein the pyrimidine analog is 5′-fluorouracil.

17. (canceled)

18. The method of claim 12, wherein the anti-Cripto antibody is a humanized anti-Cripto antibody.

19. (canceled)

20. The method of claim 12, wherein the maytansinoid is DM4.

21. The method of claim 19, wherein there is an average of 3.5 molecules of DM4 attached to one molecule of the antibody.

22. The method of claim 19, wherein the maytansoid is conjugated to the antibody via a heterobifunctional crosslinking agent.

23. The method of claim 22, wherein the heterobifunctional crosslinking agent is 4-(2-pyridyldithio)butanoic acid N-hydroxysuccinimide ester (SPDB).

24. The method of claim 12, wherein the anti-Cripto antibody conjugate and the chemotherapeutic agent are administered in a single dose, biweekly, or every three weeks.

25-29. (canceled)

30. The method of claim 12, wherein the subject is suffering from a cancer in an organ selected from the group consisting of brain, breast, testicular, colon, lung, ovary, bladder, uterine, cervical, pancreatic and stomach.

31. (canceled)

32. A method of inhibiting growth of a tumor in a subject, comprising the steps of: (i) selecting a patient having an established tumor; and (ii) administering to the subject an effective dose of an anti-Cripto antibody conjugated to a maytansoid; thereby inhibiting growth of a tumor in the subject.

33-39. (canceled)

40. The method of claim 32, wherein the anti-Cripto antibody conjugate is administered in a single dose, biweekly, or every three weeks.

41-45. (canceled)

46. The method of claim 32, wherein the anti-Cripto antibody conjugate is administered intraperitoneally, orally, intranasally, subcutaneously, intramuscularly, topically, or intravenously.

47. The method of claim 32, wherein the subject is suffering from a cancer in an organ selected from the group consisting of brain, breast, testicular, colon, lung, ovary, bladder, uterine, cervical, pancreatic and stomach.

48-66. (canceled)


SEQUENCE LISTING (71 sequences)

SEQ ID NO: 1 - DNA, 1095 bp, Artificial Sequence
  synthetic construct
caggtccaactgcagcaggttggggctgaactggtgaagcctggggcttcagtgaagctgtcctgcaaggcttctggctacaccttcaccagctactggatacactgggtgaagcagaggcctggacagggccttgagtggattggagagaatgatcctagcaacggtcgtactaactacaatgagaagttcaagaacaaggccacactgactgtagacaaatcctccagcacagcctacatgcatctcagcagcctgacatctgaggactctgcggtctattactgttcaaggggccctaattacttctattctatggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagagcccaaatcttgtgacacacctcccccatgcccacggtgcccagcacctggaggtggctcgagtggaggcggttccggagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 2 - DNA, 657 bp, Artificial Sequence
  synthetic construct
gattttttgatgacccaaactccactctccctgcctgtcagtcttggagatcaagcctccatctcttgcagatcaagtcagagcattgtacatagtaatggaaacacctatttagaatggtacctgcagaaaccaggccagtctccaaagctcctcatctacaaagtttccaaccgattttctggggtcccagacaggttcagtggcagtggatcagggacagatttcacactcaagatcagcagagtggaggctgaggatctgggagtttattactgctttcaaggttcacatgttcctctcacgttcggtgctgggaccaagctggagctgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 3 - protein, 365 aa, Artificial Sequence
  synthetic construct
QVQLQQVGAELVKPGASVKLSCKASGYTFTSYWIHWVKQRPGQGLEWIGENDPSNGRTNYNEKFKNKATLTVDKSSSTAYMHLSSLTSEDSAVYYCSRGPNYFYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPEPKSCDTPPPCPRCPAPGGGSSGGGSGGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 219 aa, Artificial Sequence
  synthetic construct
DFLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 42 aa, Artificial Sequence
  synthetic construct
EPKSCDKTHTCPPCPEPKSCDTPPPCPRCPAPGGGSSGGGSG

SEQ ID NO: 6 - protein, 188 aa, Artificial Sequence
  synthetic construct
MDCRKMARFSYSVIWIMAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 7 - protein, 188 aa, Artificial Sequence
  synthetic construct
MDCRKMVRFSYSVIWIMAISKAFELGLVAGLGHQEFARPSRGDLAFRDDSIWPQEEPAIRPRSSQRVLPMGIQHSKELNRTCCLNGGTCMLESFCACPPSFYGRNCEHDVRKENCGSVPHDTWLPKKCSLCKCWHGQLRCFPQAFLPGCDGLVMDEHLVASRTPELPPSARTTTFMLAGICLSIQSYY

SEQ ID NO: 8 - protein, 10 aa, Artificial Sequence
  synthetic construct
GGGSSGGGSG

SEQ ID NO: 9 - protein, 16 aa, Artificial Sequence
  synthetic construct
RSSQSIVHSNGNTYLE

SEQ ID NO: 10 - protein, 7 aa, Artificial Sequence
  synthetic construct
KVSNRFS

SEQ ID NO: 11 - protein, 9 aa, Artificial Sequence
  synthetic construct
FQGSHVPLT

SEQ ID NO: 12 - protein, 5 aa, Artificial Sequence
  synthetic construct
SYWIH

SEQ ID NO: 13 - protein, 17 aa, Artificial Sequence
  synthetic construct
ENDPSNGRTNYNEKFKN

SEQ ID NO: 14 - protein, 10 aa, Artificial Sequence
  synthetic construct
GPNYFYSMDY

SEQ ID NO: 15 - protein, 10 aa, Artificial Sequence
  synthetic construct
IGKTISKKAK

SEQ ID NO: 16 - protein, 15 aa, Artificial Sequence
  synthetic construct
CPEPKSCDTPPPCPR

SEQ ID NO: 17 - protein, 10 aa, Artificial Sequence
  synthetic construct
EPKSCDKTHT

SEQ ID NO: 18 - protein, 5 aa, Artificial Sequence
  synthetic construct
CPPCP

SEQ ID NO: 19 - protein, 8 aa, Artificial Sequence
  synthetic construct
APELLGGP

SEQ ID NO: 20 - protein, 12 aa, Artificial Sequence
  synthetic construct
ELKTPLGDTTHT

SEQ ID NO: 21 - protein, 20 aa, Artificial Sequence
  synthetic construct
CPRCPEPKSCDTPPPCPRCP

SEQ ID NO: 22 - protein, 7 aa, Artificial Sequence
  synthetic construct
ESKYGPP

SEQ ID NO: 23 - protein, 5 aa, Artificial Sequence
  synthetic construct
CPSCP

SEQ ID NO: 24 - protein, 8 aa, Artificial Sequence
  synthetic construct
APEFLGGP

SEQ ID NO: 25 - protein, 24 aa, Artificial Sequence
  synthetic construct
EPKSCDKTHTCPPCGGGSSGGGSG

SEQ ID NO: 26 - protein, 40 aa, Artificial Sequence
  synthetic construct
EPKSCDKTHTCPPCPEPKSCDTPPPCPRCPGGGSSGGGSG

SEQ ID NO: 27 - protein, 24 aa, Artificial Sequence
  Synthetic construct
EPKSCDKTHTCPPCGGGSSGGGSG

SEQ ID NO: 28 - protein, 25 aa, Artificial Sequence
  synthetic construct
EPKSCDKTHTSPPCPGGGSSGGGSG

SEQ ID NO: 29 - protein, 27 aa, Artificial Sequence
  synthetic construct
EPKSCDKTHTSPPCPAPGGGSSGGGSG

SEQ ID NO: 30 - protein, 25 aa, Artificial Sequence
  synthetic construct
EPKSCDKTHTCPPSPGGGSSGGGSG

SEQ ID NO: 31 - protein, 27 aa, Artificial Sequence
  synthetic construct
EPKSCDKTHTCPPSPAPGGGSSGGGSG

SEQ ID NO: 32 - protein, 27 aa, Artificial Sequence
  synthetic construct
EPKSCDKTHTCPPCPAPGGGSSGGGSG

SEQ ID NO: 33 - protein, 25 aa, Artificial Sequence
  synthetic construct
EPKSCDKTHTCPPCPGGGSSGGGSG

SEQ ID NO: 34 - protein, 29 aa, Artificial Sequence
  synthetic construct
ESKYGPPCPSCPEPKSCDTPPPCPRCPAP

SEQ ID NO: 35 - protein, 15 aa, Artificial Sequence
  synthetic construct
CPEPKSCDTPPPCPR

SEQ ID NO: 36 - protein, 13 aa, Artificial Sequence
  synthetic construct
EPKSCDKTHTCPP

SEQ ID NO: 37 - protein, 5 aa, Artificial Sequence
  synthetic construct
ESKYG

SEQ ID NO: 38 - protein, 5 aa, Artificial Sequence
  synthetic construct
PPCPS

SEQ ID NO: 39 - protein, 112 aa, Artificial Sequence
  synthetic construct
DFLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 40 - protein, 121 aa, Artificial Sequence
  synthetic construct
QVQLQQVGAELVKPGASVKLSCKASGYTFTSYWIHWVKQRPGQGLEWIGENDPSNGRTNYNEKFKNKATLTVASPKSSSTAYMHLSSLTSEDSAVYYCSRGPNYFYSMDYWGQGTSVTVSS

SEQ ID NO: 41 - protein, 113 aa, Artificial Sequence
  synthetic construct
DVVMTQTPLSLPVSLGDQASISCRSSQSLVHSNGNTYLEWYLQKPGQSPKLLIYKVSNRFSGVPDRFSGSGSGTDFTLKISRVEAEDLGVYYCFQGTHVPPYTFGGGTKLEIK

SEQ ID NO: 42 - protein, 129 aa, Artificial Sequence
  synthetic construct
QVQLQQPGAELVKPGASVKLSCKASGYTFTSYWMHWVKQRPGQGLEWIGRIDPNSGGTNYNEKFKSKATLTVASPKSSSTAYMQLSSLTSEDSAVYYCARYYYGGSSXXVYXYWYFDYWGQGTTVTVSS

SEQ ID NO: 43 - protein, 114 aa, Artificial Sequence
  synthetic construct
DIVMTQSPLSLPVTPGEPASISCRSSQSLLHSXDGNNYLNWYLQKPGQSPQLLIYLVSNRASGVPDRFSGSGSGTDFTLKISRVEAEDVGVYYCMQALQXPRXTFGQGTKVEIK

SEQ ID NO: 44 - protein, 131 aa, Artificial Sequence
  synthetic construct
QVQLVQSGAEVKKPGASVKVSCKASGYTFTSYAISWVARGQAPGQGLEWMGWINPYGNGDTNYAQKFQGRVTITADTSTSTAYMELSSLRSEDTAVYYCARAPGYGSGGGCYRGDYXFDYWGQGTLVTVSS

SEQ ID NO: 45 - protein, 112 aa, Artificial Sequence
  synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 46 - protein, 122 aa, Artificial Sequence
  synthetic construct
EVQLVESGAEVKKPGASVKVSCKASGYTFTGYFMHWVARGQAPGQGLEWMGRINPNSGGTNYAQKFQGRVTLTRDTSISTAYMELSRLRSDDTAVYYCARLDTAIDAFDIWGQGTMVTVCSN

SEQ ID NO: 47 - protein, 112 aa, Artificial Sequence
  synthetic construct
DFVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIK

SEQ ID NO: 48 - protein, 123 aa, Artificial Sequence
  synthetic construct
EVQLVESGAEVKKPGASVKVSCKASGYTFTSYWIHWVARGQAPGQGLEWIGENDPSNGRTNYNEKFKNRATLTVASPKSISTAYMELSRLRSDDTAVYYCSRGPNYFYSMDYWGQGTMVTVSS

SEQ ID NO: 49 - protein, 123 aa, Artificial Sequence
  synthetic construct
EVQLVESGAEVKKPGASVKVSCKASGYTFTSYWIHWVARGQAPGQGLEWMGENDPSNGRTNYNEKFKNRVTLTVASPTSISTAYMELSRLRSDDTAVYYCARGPNYFYSMDYWGQGTMVTVSS

SEQ ID NO: 50 - protein, 112 aa, Artificial Sequence
  synthetic construct
DFVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPLTFGAGTKLEIK

SEQ ID NO: 51 - protein, 123 aa, Artificial Sequence
  synthetic construct
QVQLVESGAEVKKPGASVKVSCKASGYTFTSYWIHWVARGQAPGQGLEWIGENDPSNGRTNYNEKFKNRVTLTVASPTSISTAYMHLSSLRSDDTAVYYCARGPNYFYSMDYWGQGTMVTVSS

SEQ ID NO: 52 - protein, 223 aa, Artificial Sequence
  synthetic construct
DVVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVALAAPSVFIFPPSDEQLKSGTASVVCLLNNFYPREAKVQWKVASPNALQSGNSQESVTEQDSKDSTYSLSSTLTLSKADYEKHKVYACEVTHQGLSSPVTKSFNRGEC

SEQ ID NO: 53 - protein, 223 aa, Artificial Sequence
  synthetic construct
DFVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVALAAPSVFIFPPSDEQLKSGTASVVCLLNNFYPREAKVQWKVASPNALQSGNSQESVTEQDSKDSTYSLSSTLTLSKADYEKHKVYACEVTHQGLSSPVTKSFNRGEC

SEQ ID NO: 54 - protein, 223 aa, Artificial Sequence
  synthetic construct
DFVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPLTFGAGTKLEIKRTVALAAPSVFIFPPSDEQLKSGTASVVCLLNNFYPREAKVQWKVASPNALQSGNSQESVTEQDSKDSTYSLSSTLTLSKADYEKHKVYACEVTHQGLSSPVTKSFNRGEC

SEQ ID NO: 55 - protein, 460 aa, Artificial Sequence
  synthetic construct
EVQLVESGAEVKKPGASVKVSCKASGYTFTSYWIHWVARGQAPGQGLEWMGENDPSNGRTNYNEKFKNRVTLTRDTSISTAYMELSRLRSDDTAVYYCARGPNYFYSMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPAPELLGGPSVFLFPPKPKDTLMISRTPEVTCVVVASPVSHEDPEVKFNWYVASPGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 56 - protein, 462 aa, Artificial Sequence
  synthetic construct
EVQLVESGAEVKKPGASVKVSCKASGYTFTSYWIHWVARGQAPGQGLEWIGENDPSNGRTNYNEKFKNRATLTVASPKSISTAYMELSRLRSDDTAVYYCSRGPNYFYSMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPAPELLGGPSVFLFPPKPKDTLMISRTPEVTCVVVASPVSHEDPEVKFNWYVASPGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 57 - protein, 462 aa, Artificial Sequence
  synthetic construct
EVQLVESGAEVKKPGASVKVSCKASGYTFTSYWIHWVARGQAPGQGLEWMGENDPSNGRTNYNEKFKNRVTLTVASPTSISTAYMELSRLRSDDTAVYYCARGPNYFYSMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPAPELLGGPSVFLFPPKPKDTLMISRTPEVTCVVVASPVSHEDPEVKFNWYVASPGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 58 - protein, 462 aa, Artificial Sequence
  synthetic construct
QVQLVESGAEVKKPGASVKVSCKASGYTFTSYWIHWVARGQAPGQGLEWIGENDPSNGRTNYNEKFKNRVTLTVASPTSISTAYMHLSSLRSDDTAVYYCARGPNYFYSMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPAPELLGGPSVFLFPPKPKDTLMISRTPEVTCVVVASPVSHEDPEVKFNWYVASPGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 59 - protein, 373 aa, Artificial Sequence
  synthetic construct
EVQLVESGAEVKKPGASVKVSCKASGYTFTSYWIHWVARGQAPGQGLEWMGENDPSNGRTNYNEKFKNRVTLTRDTSISTAYMELSRLRSDDTAVYYCARGPNYFYSMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPEPKSCDTPPPCPRCPAPGGGSSGGGSGGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 60 - protein, 375 aa, Artificial Sequence
  synthetic construct
EVQLVESGAEVKKPGASVKVSCKASGYTFTSYWIHWVARGQAPGQGLEWIGENDPSNGRTNYNEKFKNRATLTVASPKSISTAYMELSRLRSDDTAVYYCSRGPNYFYSMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPEPKSCDTPPPCPRCPAPGGGSSGGGSGGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 61 - protein, 375 aa, Artificial Sequence
  synthetic construct
EVQLVESGAEVKKPGASVKVSCKASGYTFTSYWIHWVARGQAPGQGLEWMGENDPSNGRTNYNEKFKNRVTLTVASPTSISTAYMELSRLRSDDTAVYYCARGPNYFYSMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPEPKSCDTPPPCPRCPAPGGGSSGGGSGGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 62 - protein, 375 aa, Artificial Sequence
  synthetic construct
QVQLVESGAEVKKPGASVKVSCKASGYTFTSYWIHWVARGQAPGQGLEWIGENDPSNGRTNYNEKFKNRVTLTVASPTSISTAYMHLSSLRSDDTAVYYCARGPNYFYSMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPEPKSCDTPPPCPRCPAPGGGSSGGGSGGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 63 - protein, 133 aa, Artificial Sequence
  synthetic construct
MKLPVARGLLVLMFWIPASSSDVVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIK

SEQ ID NO: 64 - protein, 133 aa, Artificial Sequence
  synthetic construct
MKLPVARGLLVLMFWIPASSSDFVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIK

SEQ ID NO: 65 - protein, 133 aa, Artificial Sequence
  synthetic construct
MKLPVARGLLVLMFWIPASSSDFVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPLTFGAGTKLEIK

SEQ ID NO: 66 - protein, 144 aa, Artificial Sequence
  synthetic construct
MGWSLILLFLVALAVALATRVLSEVQLVESGAEVKKPGASVKVSCKASGYTFTSYWIHWVARGQAPGQGLEWMGENDPSNGRTNYNEKFKNRVTLTRDTSISTAYMELSRLRSDDTAVYYCARGPNYFYSMDYWGQGTMVTVSS

SEQ ID NO: 67 - protein, 146 aa, Artificial Sequence
  synthetic construct
MGWSLILLFLVALAVALATRVLSEVQLVESGAEVKKPGASVKVSCKASGYTFTSYWIHWVARGQAPGQGLEWIGENDPSNGRTNYNEKFKNRATLTVASPKSISTAYMELSRLRSDDTAVYYCSRGPNYFYSMDYWGQGTMVTVSS

SEQ ID NO: 68 - protein, 146 aa, Artificial Sequence
  synthetic construct
MGWSLILLFLVALAVALATRVLSEVQLVESGAEVKKPGASVKVSCKASGYTFTSYWIHWVARGQAPGQGLEWMGENDPSNGRTNYNEKFKNRVTLTVASPTSISTAYMELSRLRSDDTAVYYCARGPNYFYSMDYWGQGTMVTVSS

SEQ ID NO: 69 - protein, 146 aa, Artificial Sequence
  synthetic construct
MGWSLILLFLVALAVALATRVLSQVQLVESGAEVKKPGASVKVSCKASGYTFTSYWIHWVARGQAPGQGLEWIGENDPSNGRTNYNEKFKNRVTLTVASPTSISTAYMHLSSLRSDDTAVYYCARGPNYFYSMDYWGQGTMVTVSS

SEQ ID NO: 70 - protein, 251 aa, Artificial Sequence
  synthetic construct
ASTKGPSVFPLAPSSKSTSGGTAALGCLVKDYFPEPVTVSWNSGALTSGVHTFPAVLQSSGLYSLSSVVTVPSSSLGTQTYICNVASNHKPSNTKVASPKKVEPKSCDKTHTCPPCPEPKSCDTPPPCPRCPAPGGGSSGGGSGQPREPQVYTLPPSRDELTKNQVSLTCLVKGFYPSDIAVEWESNGQPENNYKTTPPVLDSDGSFFLYSKLTVASPKSRWQQGNVFSCSVMHEALHNHYTQKSLSLSPG

SEQ ID NO: 71 - protein, 913 aa, Artificial Sequence
  synthetic construct
ALASERTHRLYSGLYPRSERVALPHEPRLEALAPRSERSERLYSSERTHRSERGLYGLYTHRALAALALEGLYCYSLEVALLYSASPTYRPHEPRGLPRVALTHRVALSERTRPASNSERGLYALALETHRSERGLYVALHISTHRPHEPRALAVALLEGLNSERSERGLYLETYRSERLESERSERVALVALTHRVALPRSERSERSERLEGLYTHRGLNTHRTYRILECYSASNVALASNHISLYSPRSERASNTHRLYSVALASPLYSLYSVALGLPRLYSSERCYSASPLYSTHRHISTHRCYSPRPRCYSPRALAPRGLLELEGLYGLYPRSERVALPHELEPHEPRPRLYSPRLYSASPTHRLEMETILESERARGTHRPRGLVALTHRCYSVALVALVALASPVALSERHISGLASPPRGLVALLYSPHEASNTRPTYRVALASPGLYVALGLVALHISASNALALYSTHRLYSPRARGGLGLGLNTYRASNSERTHRTYRARGVALVALSERVALLETHRVALLEHISGLNASPTRPLEASNGLYLYSGLTYRLYSCYSLYSVALSERASNLYSALALEPRALAPRILEGLLYSTHRILESERLYSALALYSGLYGLNPRARGGLPRGLNVALTYRTHRLEPRPRSERARGASPGLLETHRLYSASNGLNVALSERLETHRCYSLEVALLYSGLYPHETYRPRSERASPILEALAVALGLTRPGLSERASNGLYGLNPRGLASNASNTYRLYSTHRTHRPRPRVALLEASPSERASPGLYSERPHEPHELETYRSERLYSLETHRVALASPLYSSERARGTRPGLNGLNGLYASNVALPHESERCYSSERVALMETHISGLALALEHISASNHISTYRTHRGLNLYSSERLESERLESERPRGLY